SEQUENCE LISTING (91 sequences)

SEQ ID NO: 1 - protein, 155 aa, synthetic construct
  REGION 1..155: note = Description of sequence: VHH Sequence
EQVLVESGGGLVCAGGSLRLSCAASGGTFSRYMMGWFRQAPGBEREFVASIARSGGGTYFADSVKGRLTATRDDAKNTMHLQMNSLKPEDTAVYYCAAGAGGSYPALIDEEYDVWGQGIQVTVSSAAASGSLEQKLISEEDLNGAAHHHHHHGAA

SEQ ID NO: 2 - protein, 155 aa, synthetic construct
  REGION 1..155: note = Description of sequence: VHH Sequence
EQVLVESGGGLVQAGGSLRLSCTASGGIFSSYVMGWFRQAPGKDEEFVAAISKGGAATNYHESVKGRFIISRDNDKNIVYLQMNSLKPEDTAVYYCTADPLRISGATCSSPPSFWGQGIQVTVSSAAASGSLEQKLISEEDLNGAAHHHHHHGAA

SEQ ID NO: 3 - protein, 153 aa, synthetic construct
  REGION 1..153: note = Description of sequence: VHH Sequence
EQVLVESGGGLVQAGNSLRLSCSASGRIVRDYVMGWFRQAAPGHEREFVARVSRSGSTTNYADSVRGRFTISRDNARNTIYLQMDSLKPEDTAVYYCTADRIRVAPDINLDYWGQGIQVTVSSAAASGSLEQKLISEEDLNGAAHHHHHHGAA

SEQ ID NO: 4 - protein, 152 aa, synthetic construct
  REGION 1..152: note = Description of sequence: VHH Sequence
EQVLVESGGGLVQAGNSLRLSCIASGRTVSNYVMGWFRQAPGREREFVAAVSRSGSTTNYAESVRGRFTISRLGAKNNTIYLQMDSLKPEDTAVYYCAADQTRVFSDTNLDWGQGIQVTVSSAAASGSLEQKLISEEDLNGAAHHHHHHGAA

SEQ ID NO: 5 - protein, 150 aa, synthetic construct
  REGION 1..150: note = Description of sequence: VHH Sequence
EQVLVESGGGLVQAGGSLRLSCTASGGTFFTTIPMGWFRQAPGKEFEFVAAISRSGITTRYGDSVKGRFTISRDNGKNIVYLQMRSLKPEDTAVYRCAADCHRLDDGSQWGQGIQVTVSSAAASGSLEQKLISEEDLNGAAHHHHHHGAA

SEQ ID NO: 6 - protein, 152 aa, synthetic construct
  REGION 1..152: note = Description of sequence: VHH Sequence
EQVLVESGGGLVQAGGSLRLSCSVSGSTLSSYPMGWFRQAPGBEREWVSAISRSGETTAYRDSVKGRFTISRDNVKSIAYLQMNNLKPEDTAVYYCTADRLRIDRSGENDYWGQGIQVTVSSAAASGSLEQKLISEEDLNGAAHHHHHHGAA

SEQ ID NO: 7 - protein, 152 aa, synthetic construct
  REGION 1..152: note = Description of sequence: VHH Sequence
EQVLVESGGGLVQAGGSLRLSCSVSGSTLSSYPMGWFRQAPGBEREWVSAISRSGETTAYRDSVRGRFTISRDNVKSIAYLQMNNLKPEDTAVYYCTACRLRIDRSGENDYWGQGIQVTVSSAAASGSLEQKLISEEDLNGAAHHHHHHGAA

SEQ ID NO: 8 - protein, 149 aa, synthetic construct
  REGION 1..149: note = Description of sequence: VHH Sequence
EQVLVESGGGLVQPGGSLRLSCAASGFTFSDQPMGWYRQAPGQGREWIAFIAFISSGSGGTTDYRBSVRGRFTISRDNAKNIVYLQMNSLKEDDTARYYCNFRIDNKYWGQGIQVTVSSAAASGSLEQKLISEEDLNGAAHHHHHHGAA

SEQ ID NO: 9 - protein, 145 aa, synthetic construct
  REGION 1..145: note = Description of sequence: VHH Sequence
EQVLVESGGGLVQPGGSLRLSDAASGFTFDQFMGWYRQAPGQGREWIAFISSGSGGIIDYRNSVKGRFTISRDNAKNIVYLQMNSLKFDDTARYYCNFRIDNNYWGQGIQVTVSSAAASGSLEQKLISEEDLNGAAHHHHHHGAA

SEQ ID NO: 10 - protein, 145 aa, synthetic construct
  REGION 1..145: note = Description of sequence: VHH Sequence
EQVLVESGGGLVQPGGSLRLSCAASGFTFSDQPMGWYRWAPGQREWIAFTSSGSGGIIDYRNSVRGRFTISRDNAKNIVYKQMNSLKPDDTARYYCNFRIDNNYWGQGIQVTVSSAAASGSLEQKLISEEDLNGAAHHHHHHGAA

SEQ ID NO: 11 - protein, 150 aa, synthetic construct
  REGION 1..150: note = Description of sequence: VHH Sequence
EQVLVESGGDLVQDGGSLRLSCAASGGDFRNPVIGWYRQAPGRQRELVGLIISDGRTNYGDSVMGRFISMDIAKNIMYLQMNSLKPEDSAVYYCAASLLYGGIQFSTNIWGQGIQVTVSSAAASGSLEQKLISEEDLNGAAHHHHHHGAA

SEQ ID NO: 12 - protein, 151 aa, synthetic construct
  REGION 1..151: note = Description of sequence: VHH Sequence
EVQLVESGGDLVQDGGSLRLSCAASGGDFRNFVIGWYRQAPGNQRELVGLIISDGRTNYGDSVMGRFTISMDTAKNIMYLQMNSLKPEDSAVYYCAASLLYGGIQFSTNIWGQGIQVTVSSAAASGSLEQKLISEEDLNGAAHHHHHHGAA

SEQ ID NO: 13 - protein, 165 aa, synthetic construct
  REGION 1..165: note = Description of sequence: VHH Sequence
EVQVLVESGGGLVQAGGSLRLSCAVSQBIFSSSAMGWFRQAPGKEREFVTGVSWSGGRIHYADYADSVWGRFTISRDAARNAVYLQMSSLKPQDSAVYICASGREVSSSAIKYYIDPKRYDSWGQGTQTQVTVSSAAASGSLEQKLVSEEDLNGAAHHHHHHGAA

SEQ ID NO: 14 - protein, 155 aa, synthetic construct
  REGION 1..155: note = Description of sequence: VHH Sequence
EVQVLVESGGGLVQAGGSLRLSCAASGGTFKDYFVTWFRQVPGKEREFVAALSMCALPYYADSVWGRFTISGDNARSTVFLQMNSLKPEDIARYYCAAQPVGGLRDYSDYPYWGQGTQTQVTVSSAAASGSLEQKLVSEEDLNGAAHHHHHHGAA

SEQ ID NO: 15 - protein, 156 aa, synthetic construct
  REGION 1..156: note = Description of sequence: VHH Sequence
EVQVLVESGGGLVQAGGSLRLSCAASGGTFQDYPVIWFRQVPGKEREFVAPVSWLVALPYYADSVEGRFTISRDNARSTVFLQMNSLKPEDIARYYCAAQPVGGLRDYSDYPYWGQGTQTQVTVSSAAASGSLEQKLVSEEDLNGAAHHHHHHGAA

SEQ ID NO: 16 - protein, 156 aa, synthetic construct
  REGION 1..156: note = Description of sequence: VHH Sequence
EVQVLVESGGGLVQAGGSLRLSCAASGGTFKDYFVIWFRQVPGKEREFVAAISNDIALPYYADFVEGRFTISRDNAKSTVFLQMNSLKPEDIARYYCAAQPVGGLRDYSDYPYWGQGTQTQVTVSSAAASGSLEQKLVSEEDLNGAAHHHHHHGAA

SEQ ID NO: 17 - protein, 156 aa, synthetic construct
  REGION 1..156: note = Description of sequence: VHH Sequence
EVQVLVESGGGLVQAGGSLRLSCIGSBTFGSYAMGWFRQAPGKEREIVAAISRIGGNIYYPDSVKGRFTVSGDNARNIVYLQMNSLKPEDIAVYYCAASTGYIQTAVKNEYDYWGQGTQTQVTVSSAAASGSLEQKLVSEEDLNGAAHHHHHHGAA

SEQ ID NO: 18 - protein, 157 aa, synthetic construct
  REGION 1..157: note = Description of sequence: VHH Sequence
EVQVLVESGGGLVQPGGSLRLSCIGSGRAFSSYAMGWFRQAPGKEREIVAAISRIGGNIYYPDSVKGRFIVSGDNARNTVYLQMNSLKPEDIAVYYCAASTRYIQTAVQKEYDFWGQGTQTQVTVSSAAASGSLEQKLVSEEDLNGAAHHHHHHGAA

SEQ ID NO: 19 - protein, 157 aa, synthetic construct; identical to SEQ ID NO: 18
  REGION 1..157: note = Description of sequence: VHH Sequence

SEQ ID NO: 20 - protein, 156 aa, synthetic construct
  REGION 1..156: note = Description of sequence: VHH Sequence
EVQVLVKSGGGLVQAGGSLRLSCIGSGRIFSGYAMGWFRQAPGKEREIVAAISRIGGNYYPDSVKGRFIVSGDNARNTVYLQMNSLKPEDIAVYYCAASTRYIQTAVQKEYDFWGQGTQTQVTVSSAAASGSLEQKLVSEEDLNGAAHHHHHHGAA

SEQ ID NO: 21 - protein, 133 aa, synthetic construct
  REGION 1..133: note = Description of sequence: VHH Sequence
EVQVLVESGGGLVQAGGSLRLSCTASGIIFSIRAAGWYRQAPGRQREWIGSIAADGITNYTPSLQMNSLKPEDIAVYYCAADAVAYGSKYWGQGTQTQVTVSSAAASGSLEQKLVSEEDLNGAAHHHHHHGAA

SEQ ID NO: 22 - protein, 150 aa, synthetic construct
  REGION 1..150: note = Description of sequence: VHH Sequence
EVQVLVESGGGLVQPGGSLRSCAAGFASSVATMSWVRQAPGKGLEWVSIINGGLGITSYADSVKGRFTISRDNARNLYLQMNSLKPEDIAVYYQTIGAGRPANPPPWRGQGTQTQVTVSSAAASGSLEQKLVSEEDLNGAAHHHHHHGAA

SEQ ID NO: 23 - protein, 152 aa, synthetic construct
  REGION 1..152: note = Description of sequence: VHH Sequence
EVQVLVESGGGLVQPGGSLRSCAASGFAFSVATMSWVRQAPGKGFEWVSIISGAVGVISYADSVKGRFTISRQKAKNILYLQMNSLKPEDIAVYFQTIGAGRPANLPPWRGQGTQTQVTVSSAAASGSLEQKLVSEEDLNGAAHHHHHHGAA

SEQ ID NO: 24 - protein, 148 aa, synthetic construct
  REGION 1..148: note = Description of sequence: VHH Sequence
EVQVLVESGGGLVQPGGSLRSCTASGFTGSSDWMYWVRQAPGKGLEWVAGVKVNSSGYTHKYASFVKGRFTISRDNAKNTLYLQMNLKSEDIAVYYCAKDMRGDGKGQGTQTQVTVSSAAASGSLEQKLVSEEDLNGAAHHHHHHGAA

SEQ ID NO: 25 - protein, 148 aa, synthetic construct
  REGION 1..148: note = Description of sequence: VHH Sequence
EVQVLVESGGGLVQPGGSLRTCAASGPITSSCDWMYWVRQAPGKGLEWVSGINRGGGTQKYADFVKGRFTISRDNAKNILYLQMNSDKSEDSAVYYCAKDMNGSSKGQGTQTQVTVSSAAASGSLEQKLVSEEDLNGAAHHHHHHGAA

SEQ ID NO: 26 - protein, 157 aa, synthetic construct
  REGION 1..157: note = Description of sequence: VHH Sequence
EVQVLVESGGGLVQPGGSLRSCAASGPILDDYAVGWFRQAPGKEREWISGISSRDGSIVYADSVKGRFTISRDNAKNIYYLQMNSLKPEDIAVYYCAAAVGYILTAVKSEYNDWSGQGTQTQVTVSSAAASGSLEQKLVSEEDLNGAAHHHHHHGAA

SEQ ID NO: 27 - protein, 155 aa, synthetic construct
  REGION 1..155: note = Description of sequence: VHH Sequence
EVQLVESGGGSVQAGGSLRLSCIASIRAGNRYAMGWFRQAPGKEREFVAGISWSGGNTYHADSVWGRFTISRDNARNTLYLIMNSLRSEDIAVYYCAASWKFLTFRGDDYIYWGQGTQTQVTVSSAAASGSLEQKLVSEEDLNGAAHHHHHHGAA

SEQ ID NO: 28 - protein, 158 aa, synthetic construct
  REGION 1..158: note = Description of sequence: VHH Sequence
EVQLVESGGGLVQPGGSLRLSCIASGSSEDYYAIGWFRQAPGHEREGVSCISSRDGRIYWVDSAKGRFTISRDNGKNTVYLQMNSLKPEDIAVYYCATSRWSYCPSDWSFVPSEFGSWGQGIQVTVSSAAASGSLEQKLISEEDLNGAAHHHHHHGAA

SEQ ID NO: 29 - protein, 158 aa, synthetic construct
  REGION 1..158: note = Description of sequence: VHH Sequence
EVQLVESGGGLVQPGGSLRLSCIASGSSEDYYAIGWFRQAPGHEREGVSCISSRDGRIYWADSVEGRFIISRDNGKNTVYLQMNSLKPEDIAVYYCATSRWSFCPSDWSRVPSFFGSWGQGIQVTVSSAAASGSLEQKLISEEDLNGAAHHHHHHGAA

SEQ ID NO: 30 - protein, 158 aa, synthetic construct
  REGION 1..158: note = Description of sequence: VHH Sequence
EVQLVESGGGLVQPGGSLRLSCIASGSSEDYYAIGWFRQAPGHEREGVSCISSRDGRIYWADSVEGRFIISRDNGKNTVYLQMNSLKPEDIAVYYCATSRWSFCPSDWSRVPSPFGSWGQGIQVTVSSAAASGSLEQKLISEEDLNGAAHHHHHHGAA

SEQ ID NO: 31 - protein, 158 aa, synthetic construct; identical to SEQ ID NO: 30
  REGION 1..158: note = Description of sequence: VHH Sequence

SEQ ID NO: 32 - protein, 158 aa, synthetic construct; identical to SEQ ID NO: 30
  REGION 1..158: note = Description of sequence: VHH Sequence

SEQ ID NO: 33 - protein, 146 aa, synthetic construct
  REGION 1..146: note = Description of sequence: VHH Sequence
EVQLVESGGGLVQAGGSLRLSCAASGSTLSISVMGWFRQAPGHRREFVAAIGRGGSIKVADSVKGRFIISRDNAKNTVYLQMNSLKPEDIAVYSCNAADYGSRYYWGQGIQVTVSSAAASGSLEQKLISEEDLNGAAHHHHHHGAA

SEQ ID NO: 34 - protein, 146 aa, synthetic construct
  REGION 1..146: note = Description of sequence: VHH Sequence
EVQLVESGGGLVQAGGSLRLSCAASGSTESISVMGWFRQAPGHRREFVAAIGRGGSIKVADSVKGRFIISRDNAKNTVYLQMNSLKPEDIAVYSCNAADYGSRYYWGQGIQVTVSSAAASGSLEQKLISEEDLNGAAHHHHHHGAA

SEQ ID NO: 35 - protein, 146 aa, synthetic construct; identical to SEQ ID NO: 34
  REGION 1..146: note = Description of sequence: VHH Sequence

SEQ ID NO: 36 - protein, 149 aa, synthetic construct
  REGION 1..149: note = Description of sequence: VHH Sequence
EVQLVESGGGLVQPGGSLRLSCAASGSAFSIKVMGWHRRAPGHQRELVAGITSGGSTRIADSVKDRFIISRDNSKNTVYLQMDNLKPEDIAVYYCRTTLYNRGATYDYWGQGIQVTVSSAAASGSLEQKLISEEDLNGAAHHHHHHGAA

SEQ ID NO: 37 - protein, 144 aa, synthetic construct
  REGION 1..144: note = Description of sequence: VHH Sequence
EVQLVESGGGLVQPGGSLRLSCAASGSIFSVNBMGWYRQAPGHRELVATMTNGGSLNYADSVRGRFIISRDNAKNTVDLQMDILKPEDIAVYFCMYRGLGSISWGQGIQVTVSSAAASGSLEQKLISEEDLNGAAHHHHHHGAA

SEQ ID NO: 38 - protein, 154 aa, synthetic construct
  REGION 1..154: note = Description of sequence: VHH Sequence
EVQLVESGGGFVQPGGSLRLSCAASGFTLDYYAVGWFRQAPGHEREGVSCISSSDSSTYYADSVKGRFIISRDNAKNTVYLQMNKLKPEDIAVYYCATCAPHCISSIYDAYHYWGQGIQVTVSSAAASGSLEQKLISEEDLNGAAHHHHHHGAA

SEQ ID NO: 39 - protein, 155 aa, synthetic construct
  REGION 1..155: note = Description of sequence: VHH Sequence
EVQLVESGGGLVQPGGSLRLSCAASGFTIDSIAITWFRQAPHEREGVSCISSSDGRIYYIDSAKGRFIISREDAKNTVYLQMNSLKPEDTAVYYCAAKRRRSWCLSSPGDYCELWGQGIQVTVSSAAASGSLEQKLISEEDLNGAAHHHHHHGAA

SEQ ID NO: 40 - protein, 148 aa, synthetic construct
  REGION 1..148: note = Description of sequence: VHH Sequence
EVQLVESGGGLVQTGTSLRLSCAASGITASDYFMAWFRQVPRKERVLVASISWSQDPYHARSMQGRFTISRDTAKNILYLQMNNLKPEDIAIYYCAABIRBGAEYDFWQGQTQVTVSSAAASGSLEQKLISEEDLNGAAHHHHHHGAA

SEQ ID NO: 41 - protein, 152 aa, synthetic construct
  REGION 1..152: note = Description of sequence: VHH Sequence
EVQLVESGGGLVQTGTSLRLSCAASGITASDYFMAWFRQVPRKERVLVASISWSQDPYHARSMGGRFTISRDTAKNILYLQMNNLKPEDIAIYYCAABIRDGILBGAEYDFWGQGTQVTVSSAAASGSLEQKLISEEDLNGAAHHHHHHGAA

SEQ ID NO: 42 - protein, 153 aa, synthetic construct
  REGION 1..153: note = Description of sequence: VHH Sequence
EVQLVESGGGLVQAGDSLRLSCAASGGTFKCYFVIWFRQVPGKEREFVAAISWLISLPYYADSVEGRFTISRDNAKSIVFLQMNSLKPEDIARYYCAAQKVGGIRDYSGYPYWGQGTQVTVSSAAASGSLEQKLISEEDLNGAAHHHHHHGAA

SEQ ID NO: 43 - protein, 154 aa, synthetic construct
  REGION 1..154: note = Description of sequence: VHH Sequence
EVQLVESGGGLVQAGGSLRLACVASHGTFSSRFAMAWFRQAPGKEREFVIAITWNGDNTYYAESVWGRFTISRDNAKNTAYDQMNSLKPEDIAVYYCAAAARKQIFISSSYNCWGQGTQVTVSSAAASGSLEQKLISEEDLNGAAHHHHHHGAA

SEQ ID NO: 44 - protein, 155 aa, synthetic construct
  REGION 1..155: note = Description of sequence: VHH Sequence
EVQLVESGGGLVQAGGSLRLACVASKGTFSSRFAMAWFRQAPGKEREFVIAITWNGDKTYYAESVWGRFTISRDNAKNTAYLQMNSLKEPEDIAVYYCAAAARKQIFISSSYNDWGQGTQVTVSSAAASGSLEQKLISEEDLNGAAHHHHHHGAA

SEQ ID NO: 45 - protein, 151 aa, synthetic construct
  REGION 1..151: note = Description of sequence: VHH Sequence
EVQLVESGGGLVQAGGSLRLSCAASGRTVSSIAMGWFRQAPGKEREAVAALSWSGSRTYYADSVWGRFTISRVNAKNTVYLQMNSLKPEDIAVYYCRAAEFTDYTGSANYWGQGTQVTVSSAAASGSLEQKLISEEDLNGAAHHHHHHGAA

SEQ ID NO: 46 - protein, 154 aa, synthetic construct
  REGION 1..154: note = Description of sequence: VHH Sequence
EVQLVESGGGLVQAGGSLRLSCTGSGRTFSGYAMGWFRQAPGKEREIVAAISRTGGNTYYPDSVWGRFIVSGDNAKNTVYLQMNSLKPEDIAVYYCAASDRYIGTVVGHSYDYWGQGTQVTVSSAAASGSLEQKLISEEDLNGAAHHHHHHGAA

SEQ ID NO: 47 - protein, 156 aa, synthetic construct
  REGION 1..156: note = Description of sequence: VHH Sequence
EVQLVESGGGLVQAGGSLRLSCAASGRTFGSSBMGWFRQAPGKEREFVAFISAGPGGIIYADSVKGRFTISRDNAKNMVYLQMDSLKEPEDIAVYYCVABPSBGSTSVRLPRYDYWGKGTQVTVSSAAASGSLEQKLISEEDLNGAAHHHHHHGAA

SEQ ID NO: 48 - protein, 147 aa, synthetic construct
  REGION 1..147: note = Description of sequence: VHH Sequence
EVQLVESGGGVVQAGGSLRLSCTSSGIYFBVWTMGWYRQSPGKQRELVASISSDGIINNADSVGGRFTISRDNSKNIVYLQMNSIEPEDIAVYYCNABPPSGATDYWGQGTQVTVSSAAASGSLEQKLISEEDLNGAAHHHHHHGAA

SEQ ID NO: 49 - protein, 161 aa, synthetic construct
  REGION 1..161: note = Description of sequence: VHH Sequence
EVQLVESGGGLVQTGGSLRLSCATSGFTFEYSAIAWFRQAPGKGREGVSCISNRDGTIVYADSVKGRFTISSDKAKNTVYLQMNSITPEDIGVYYCAREAGVYTAEYCRDSRYDPVSKEAWGKGTLVTVSSAAASGSLEQKLISEEDLNGAAHHHHHHGAA

SEQ ID NO: 50 - protein, 158 aa, synthetic construct
  REGION 1..158: note = Description of sequence: VHH Sequence
EVQLVESGGGLVQAGGSLRLSCATSGFTFGDYAIGWFRQAPGKAREGVSCLSTRDGTTYYADSVWGRFTISTDNAQNTVYLQMNSLKEPEDIAVYYCAGEQISFRAVYYCTEYEEVYWSQGTQVTVSSAAASGSLEQKLISEEDLNGAAHHHHHHGAA

SEQ ID NO: 51 - protein, 146 aa, synthetic construct
  REGION 1..146: note = Description of sequence: VHH Sequence
EVQLVESGGGLVQLGGSLRLSCAGSBIILSRFGVSWYRQAPGKQRELVAAFTRGGSTDYADSVKGRFTISRDDNCNTVYLQMNSLKEPEDIAVYYCYAPVIDRDYWGQGTQVTVSSAAASGSLEQKLISEEDLNGAAHHHHHHGAA

SEQ ID NO: 52 - protein, 146 aa, synthetic construct
  REGION 1..146: note = Description of sequence: VHH Sequence
EVQLVESGGGLVQAGGSLRLSCAASGVLISRFIKGWYRQAPGQQRELVAIVDRGGSTNYADSVRGRFTISKDNAKNTVYLQMNSLKEPEDIAVYYCNABIIDRDYWGQGTQVTVSSAAASGSLEQKLISEEDLNGAAHHHHHHGAA

SEQ ID NO: 53 - protein, 156 aa, synthetic construct
  REGION 1..156: note = Description of sequence: VHH Sequence
EVQLVESGGGLVQAGGSLRLSCAASGRTIRNYVMGWFRQAPGKEREFVSAISWNSGSTYYANSAKGRFTISRDTAKNTVYLQMNSLKPEDIAVYYCAAGPIRIGTFYDASTTYEDWGQGTQVTVSSAAASGSLEQKLISEEDLNGAAHHHHHHGAA

SEQ ID NO: 54 - protein, 149 aa, synthetic construct
  REGION 1..149: note = Description of sequence: VHH Sequence
EVQLVESGGGLVQPGGSLRLSCAASGFNINDYFMSWVRQAPGRGLEWVSGINWNGVIYYAQSMKGRFTISRDYANNTLYLQMNSLKSEDIAVYYCISYIGWGSGYMDYWGKGTLVTVSSAAASGSLEQKLISEEDLNGAAHHHHHHGAA

SEQ ID NO: 55 - protein, 145 aa, synthetic construct
  REGION 1..145: note = Description of sequence: VHH Sequence
EVQLVESGGGLVQPGGSLRLSCAASGNILSINNMAWYRQAPGKQRELVAEITNGGITNYSDSVKGRFTISRDNGQNTAYLQMNSLRPEDTAVYYCNAEIMIKGWLPTRTDNWGQGTQVTVSSAAADYKDDDDKGAAHHHHHHGAA

SEQ ID NO: 56 - protein, 143 aa, synthetic construct
  REGION 1..143: note = Description of sequence: VHH Sequence
EVQLVESGGGLVQAGGSLRVSCAASGRAVHNYALNWFRQAPGKEREFVAAISWTRRTYYANSVRGRFTISRDNNGNMVHLQMSNLKSEDTALYYCAAATTWPRLDGAEYWGQGTQVTVSSAAADYKDDDDKGAAHHHHHHGAA

SEQ ID NO: 57 - protein, 145 aa, synthetic construct
  REGION 1..145: note = Description of sequence: VHH Sequence
EVQLVESGGGLVQAGDSLRLSCLSVQRTYIAYPVAWFRQAPGRQREFVASINYNGGSTVYTDSVKGRFTISRDNAKSTAYLQMNNLEPEDTAVYTCAAAVRSNPRQGAYDTWGQGTQVTVSSAAADYKDDDDKGAAHHHHHHGAA

SEQ ID NO: 58 - protein, 145 aa, synthetic construct
  REGION 1..145: note = Description of sequence: VHH Sequence
EVQLVESGGGLVQPGGSLRLSCEAAGSILRVNSMGWYRQAPGKQRDLVAIITSNNDAKYSDSVKGRFTISRDNAKNTVYLQMSSLKPDDTAVYYCNAYVETAGWIPTTHNLWGQGTQVTVSSAAADYKDDDDKGAAHHHHHHGAA

SEQ ID NO: 59 - protein, 145 aa, synthetic construct
  REGION 1..145: note = Description of sequence: VHH Sequence
EVQLVESGGGLVQSGGSLRLSCAASGRIFSITNMGWYRQAPGEQRELVADMPSGGSTNYADSVKGRFTISRDIAKNTVYLQMNSLKPEDTAVYYCNLKMSQPGWLVTNHNFWGQGTQVTVSSAAADYKDDDDKGAAHHHHHHGAA

SEQ ID NO: 60 - protein, 152 aa, synthetic construct
  REGION 1..152: note = Description of sequence: VHH Sequence
EVQLVESGGGLVQAGGSLRLSCAASGSLSSINAMAWYRQAPGKQRDLVATITREGITNYADSVKGRFTISRDNARNAVSLQMNSLKPEDTGVYYCNADVEIPGWLPTTSRYWGQGTQVTVSSAAASGSLEQKLISEEDLNGAAHHHHHHGAA

SEQ ID NO: 61 - protein, 149 aa, synthetic construct
  REGION 1..149: note = Description of sequence: VHH Sequence
EVQLVESGGGLAQPGGSLRLSCAGSGFPLGGYAVAWFRQAPGKGREGVSCVSSNGAAFYSDAVKGRFTISRDNEAHTVYLQMNNLKPEDTALYHCATASYYCSGYRTMSRPVVGSWGQGTQVTVSSAAADYKDDDDKGAAHHHHHHGAA

SEQ ID NO: 62 - protein, 146 aa, synthetic construct
  REGION 1..146: note = Description of sequence: VHH Sequence
EVQLVESGGGLVEAGGSLTLSCTTSEPTSLLNLMGWWRQGPGKEREFVASSNWSGKLVDYADGVEGRFTVIRNEDENAISLQMNSLTVEDTAIYYCAASRLGLPRSAQAYQYWGQGTQVTVSSAAADYKDDDDKGAAHHHHHHGAA

SEQ ID NO: 63 - protein, 149 aa, synthetic construct
  REGION 1..149: note = Description of sequence: VHH Sequence
EVQLVESGGGLVQPGGSLRLSCTSGFAFDNYCMYWVRQAPGKGLEWISGITNGGSFSYYADSVKGRFTISRDNAKNTLFLEMNSLKSDDTAVYYCAIHGHGCTWDSLRTTSGPRYRGQGTQVTVSSAAADYKDDDDKGAAHHHHHHGAA

SEQ ID NO: 64 - protein, 145 aa, synthetic construct
  REGION 1..145: note = Description of sequence: VHH Sequence
EVQLVESGGGLVQTGGSLRLSCAVSGNTLRINTMGWYRQAPGKQRELVADITEVGYTNYADSVKGRFTISRDNAKNTAYLQMNSLKPEDTAVYYCNAVVQKPGWLPTNQNYWGQGTQVTVSSAAADYKDDDDKGAAHHHHHHGAA

SEQ ID NO: 65 - protein, 151 aa, synthetic construct
  REGION 1..151: note = Description of sequence: Consensus sequence
  SITE 47: note = misc_feature - Xaa can be any naturally occurring amino acid
  SITE 50: note = misc_feature - Xaa can be any naturally occurring amino acid
  SITE 53: note = misc_feature - Xaa can be any naturally occurring amino acid
  SITE 55: note = misc_feature - Xaa can be any naturally occurring amino acid
  SITE 57: note = misc_feature - Xaa can be any naturally occurring amino acid
  SITE 101: note = misc_feature - Xaa can be any naturally occurring amino acid
  SITE 104: note = misc_feature - Xaa can be any naturally occurring amino acid
  SITE 106: note = misc_feature - Xaa can be any naturally occurring amino acid
EVQLVESGGGLVQPGGSLRLSCAASGFTFDBYAMGWFRQAPGKEREXVAXISXGXGXTYYADSVKGRFTISRDNAKNTVYLQMNSLKPEDTAVYYCAASVXTAXSXSYNYWGQGTQVTVSSAAASGSLEQKLISEEDLNGAAHHHHHHGAA

SEQ ID NO: 66 - protein, 146 aa, synthetic construct
  REGION 1..146: note = Description of sequence: VHH Sequence
EVQLVESGGGLVQPGGSLRLSCAASGFTFSDQPMGWYRQAPGQGREWIAFISSGSGGTTDYKNSVKGRFTISRDNAKNIVYLQMNSLKPDDTARYYCNFRIDNNYWGQGTQVTVSSAAASGSLEQKLISEEDLNGAAHHHHHHGAA

SEQ ID NO: 67 - protein, 151 aa, synthetic construct
  REGION 1..151: note = Description of sequence: VHH Sequence
EVQLVESGGDLVQDGGSLRLSCAASGGDFRNPVTGWYRQAPGKQRELVGLITSDGRTNYGDSVMGRFTISMDTAKNTMYLQMNSLKPEDSAVYYCAASLLYGGLQFSTNIWGQGTQVTVSSAAASGSLEQKLISEEDLNGAAHHHHHHGAA

SEQ ID NO: 68 - protein, 153 aa, synthetic construct
  REGION 1..153: note = Description of sequence: VHH Sequence
EVQLVESGGDSVQAGGSLRLSCTASIRAGNRYAMGWFRQAPGKEREFVAGISWSGGNTYHADSVNGRFTISRDNAKNTLYLTMNSLKSEDTAVYYCAASWKPLTFRGDDYTYWGQGTQVTVSSAAASGSLEQKLISEEDLNGAAHHHHHHGAA

SEQ ID NO: 69 - protein, 154 aa, synthetic construct
  REGION 1..154: note = Description of sequence: VHH Sequence
EVQLVESGGGLVQPGGSLRLSCAASGFTLDDYAVGWFRQAPGKEREWLSGISSRDGSTVYADSVKGRFTISRDNAKNTYYLQMNSLKPEDTAVYYCAAAVGYILTRVKSHYNDWSQGTQVTVSSAAASGSLEQKLISEEDLNGAAHHHHHHGAA

SEQ ID NO: 70 - protein, 158 aa, synthetic construct
  REGION 1..158: note = Description of sequence: VHH Sequence
EVQLVESGGGLVQPGGSLRLSCTASGSSLDYYAIGWFRQAPGKEREGVSCISSRDGRIYWADSVEGRFTISRDNGKNTVYLQMNSLKPEDTAVYYCATSRWSFCPSDWSPVPSPFGSWGQGTQVTVSSAAASGSLEQKLISEEDLNGAAHHHHHHGAA

SEQ ID NO: 71 - protein, 161 aa, synthetic construct
  REGION 1..161: note = Description of sequence: VHH Sequence
EVQLVESGGGLVQTGGSLRLSCATSGFTFEYSAIAWFRQAPGKGREGVSCISNRDGTTVYADSVKGRFTISRDNAKNTVYLQMNSLTPEDTGVYYCAADAGVYTADYCRDSRYDPVSKDAWGKGTLVTVSSAAASGSLEQKLISEEDLNGAAHHHHHHGAA

SEQ ID NO: 72 - protein, 157 aa, synthetic construct
  REGION 1..157: note = Description of sequence: VHH Sequence
EVQLVESGGGLVQAGGSLRLSCATSGFTPGDYAIGWFRQAPGKAREGVSCLSTRDGTTYYADSVKGRFTISTDNAQNTVYLQMNSLKPEDTAVYYCAGEQISFRADYYCTEYEPVYWSQGTQVTVSSAAASGSLEQKLISEEDLNGAAHHHHHHGAA

SEQ ID NO: 73 - protein, 143 aa, synthetic construct
  REGION 1..143: note = Description of sequence: VHH Sequence
EVQLVESGGGLVQAGGSLRVSCAASGRAVHNYALNWFRQAPGKAREFVSAISWTRRTYYANSVRGRFTISRDNNGNMVHLQMSNLKSEDTALYYCAAATTWPRLDGAEYWGQGTQVTVSSAAADYKDDDDKGAAHHHHHHGAA

SEQ ID NO: 74 - protein, 145 aa, synthetic construct
  REGION 1..145: note = Description of sequence: VHH Sequence
EVQLVESGGGLVQPGGSLRLSCEAAGSILRVNSMGWYRQAPGKQRDLVSIITSNNDAKYSDSVRGRFTISRDNAKNTVYLQMSSLKPDDTAVYYCNAYVETAGWIPTTHNLWGQGTQVTVSSAAADYKDDDDKGAAHHHHHHGAA

SEQ ID NO: 75 - protein, 145 aa, synthetic construct; identical to SEQ ID NO: 59
  REGION 1..145: note = Description of sequence: VHH Sequence

SEQ ID NO: 76 - protein, 146 aa, synthetic construct
  REGION 1..146: note = Description of sequence: VHH Sequence
EVQLVESGGGLVEAGGSLTLSCTTSEPTSLLNLMGWWRQGPGKEREFVASSNWSGKLVDYADGVEGRFTVIRNEDENAISLQMNSLTVEDTAIYYCAASRLGLPRSAQANQYWGQGTQVTVSSAAADYKDDDDKGAAHHHHHHGAA

SEQ ID NO: 77 - protein, 150 aa, synthetic construct
  REGION 1..150: note = Description of sequence: VHH Sequence
EVQLVESGGGLVEPGGSLRLSCATSGFAFDNYCMYWVRQAPGKGLEWISGITNSGSFSYYADSVKGRFTISRDNAKNTLFLEMNSLTVDDTAVYYCAIHGHGCTWDSLRTTSGPRYRGQGTQVTVSSAAADYKDDDDKGAAHHHHHHGAA

SEQ ID NO: 78 - protein, 123 aa, synthetic construct
  REGION 1..123: note = Description of sequence: VHH Sequence
EVQLVESGGGLVQPGGSLRLSCAASGFTFSDQPMGWYRQAPGQGREWIAFISSGSGGTTDYKNSVKGRFTISRDNAKNIVYLQMNSLKPDDTARYYCNFRIDNNYWGQGTQVTVSSAAASGSL

SEQ ID NO: 79 - protein, 128 aa, synthetic construct
  REGION 1..128: note = Description of sequence: VHH Sequence
EVQLVESGGDLVQDGGSLRLSCAASGGDFRNPVTGWYRQAPGKQRELVGLITSDGRTNYGDSVMGRFTISMDTAKNTMYLQMNSLKPEDSAVYYCAASLLYGGLQFSTNIWGQGTQVTVSSAAASGSL

SEQ ID NO: 80 - protein, 131 aa, synthetic construct
  REGION 1..131: note = Description of sequence: VHH Sequence
EVQLVESGGGLVQAGGSLRLSCAASGGTFSRYMMGWFRQAPGKEREFVASIARSGGTYFADSVKGRLTATRDDAKNTMHLQMNSLKPEDTAVYYCAAGAGGSYPALLDFEYLVWGQGTQVTVSSAAASGSL

SEQ ID NO: 81 - protein, 131 aa, synthetic construct
  REGION 1..131: note = Description of sequence: VHH Sequence
EVQLVESGGGLVQPGGSLRLSCAASGFTLDDYAVGWFRQAPGKEREWLSGISSRDGSTVYADSVKGRFTISRDNAKNTYYLQMNSLKPEDTAVYYCAAAVGYILTRVKSHYNDWSQGTQVTVSSAAASGSL

SEQ ID NO: 82 - protein, 130 aa, synthetic construct
  REGION 1..130: note = Description of sequence: VHH Sequence
EVQLVESGGGSVQAGGSLRLSCTASIRAGNRYAMGWFRQAPGKEREFVAGISWSGGNTYHADSVNGRFTISRDNAKNTLYLTMNSLKSEDTAVYYCAASWKPLTFRGDDYTYWGQGTQVTVSSAAASGSL

SEQ ID NO: 83 - protein, 135 aa, synthetic construct
  REGION 1..135: note = Description of sequence: VHH Sequence
EVQLVESGGGLVQPGGSLRLSCTASGSSLDYYAIGWFRQAPGKEREGVSCISSRDGRIYWADSVEGRFTISRDNGKNTVYLQMNSLKPEDTAVYYCATSRWSFCPSDWSPVPSPFGSWGQGTQVTVSSAAASGSL

SEQ ID NO: 84 - protein, 138 aa, synthetic construct
  REGION 1..138: note = Description of sequence: VHH Sequence
EVQLVESGGGLVQTGGSLRLSCATSGFTFEYSAIAWFRQAPGKGREGVSCISNRDGTTVYADSVKGRFTISSDNAKNTVYLQMNSLTPEDTGVYYCAADAGVYTADYCRDSRYDPVSKDAWGKGTLVTVSSAAASGSL

SEQ ID NO: 85 - protein, 134 aa, synthetic construct
  REGION 1..134: note = Description of sequence: VHH Sequence
EVQLVESGGGLVQAGGSLRLSCATSGFTPGDYAIGWFRQAPGKAREGVSCLSTRDGTTYYADSVKGRFTISTDNAQNTVYLQMNSLKPEDTAVYYCAGEQISFRAVYYCTEYEPVYWSQGTQVTVSSAAASGSL

SEQ ID NO: 86 - protein, 120 aa, synthetic construct
  REGION 1..120: note = Description of sequence: VHH Sequence
EVQLVESGGGLVQAGGSLRVSCAASGRAVHNYALNWFRQAPGKEREFVAAISWTRRTYYANSVRGRFTISRDNNGNMVHLQMSNLKSEDTALYYCAAATTWPRLDGAEYWGQGTQVTVSS

SEQ ID NO: 87 - protein, 125 aa, synthetic construct
  REGION 1..125: note = Description of sequence: VHH Sequence
EVQLVESGGGLVQPGGSLRLSCEAAGSILRVNSMGWYRQAPGKQRDLVAIITSNNDAKYSDSVKGRFTISRDNAKNTVYLQMSSLKPDDTAVYYCNAYVETAGWIPTTHNLWGQGTQVTVSSAAA

SEQ ID NO: 88 - protein, 125 aa, synthetic construct
  REGION 1..125: note = Description of sequence: VHH Sequence
EVQLVESGGGLVQSGGSLRLSCAASGRIFSITNMGWYRQAPGEQRELVADMPSGGSTNYADSVKGRFTISRDIAKNTVYLQMNSLKPEDTAVYYCNLKMSQPGWLVTNHNFWGQGTQVTVSSAAA

SEQ ID NO: 89 - protein, 126 aa, synthetic construct
  REGION 1..126: note = Description of sequence: VHH Sequence
EVQLVESGGGLVEAGGSLTLSCTTSEPTSLLNLMGWWRQGPGKEREFVASSNWSGKLVDYADGVEGRFTVIRNEDENAISLQMNSLTVEDTAIYYCAASRLGLPRSAQAYQYWGQGTQVTVSSAAA

SEQ ID NO: 90 - protein, 130 aa, synthetic construct
  REGION 1..130: note = Description of sequence: VHH Sequence
EVQLVESGGGLVQPGGSLRLSCATSGFAFDNYCMYWVRQAPGKGLEWISGITNGGSFSYYADSVKGRFTISRDNAKNTLFLEMNSLKSDDTAVYYCAIHGHGCTWDSLRTTSGPRYRGQGTQVTVSSAAA

SEQ ID NO: 91 - protein, 135 aa, synthetic construct; identical to SEQ ID NO: 83
  REGION 1..135: note = Description of sequence: VHH Sequence

(19) United States
(12) Patent Application Publication
(10) Pub. No.: US 2025/0270298 A1
(43) Pub. Date: Aug. 28, 2025

(54) MULTIVALENT ANTI-CAMPYLOBACTER ANTIBODIES AND VACCINE

(71) Applicant: BiomEdit, LLC, Fishers, IN (US)

(72) Inventors: Arvind Kumar, Fishers, IN (US); Dharanesh Mahimapura Gangaiah, Fishers, IN (US)

(21) Appl. No.: 18/692,967

(22) PCT Filed: Sep. 20, 2022
(86) PCT No.: PCT/US2022/044131

§ 371 Date: Mar. 18, 2024

Related U.S. Application Data
(60) Provisional application No. 63/246,156, filed on Sep. 20, 2021.

(51) Int. Cl. C07K 16/12; A23K 10/18; A23K 20/147; A23K 50/75; A61K 9/00; A61K 39/00; A61K 39/40; A61P 31/04

(52) U.S. Cl. CPC C07K 16/121; A23K 10/18; A23K 20/147; A23K 50/75; A61K 9/0053; A61K 39/40; A61P 31/04; A61K 2039/505; A61K 2039/542; A61K 2039/552; C07K 2317/14; C07K 2317/569

(57) ABSTRACT

One or more antibodies, particularly nanobodies or VHH single domain antibodies, directed against one or more Campylobacter bacteria targets with roles in Campylobacter colonization, particularly Campylobacter jejuni, in animals, particularly in poultry are provided. The nanobodies are useful in reducing or blocking Campylobacter colonization or infection. The invention provides methods for reducing or blocking Campylobacter colonization or infection, for improving food safety, and for reducing bacterial gastroenteritis in animals, including humans.

### CROSS-REFERENCES TO RELATED APPLICATIONS

This application is the National Stage of International Application No. PCT/US2022/044131, filed Sep. 20, 2022, which claims priority to U.S. Provisional Application No. 63/245,156 filed Sep. 20, 2021, the contents of each are hereby incorporated by reference herein.

### REFERENCE TO A SEQUENCE LISTING

The instant application contains a Sequence Listing encoded as ASCII text which was filed electronically by EFS-web and is hereby incorporated by reference in its entirety. Said ASCII test copy of the Sequence Listing, created on Sep. 20, 2022, is named “2950-6-PCT_ST26” and is 112,545 bytes in size.

### FIELD OF THE INVENTION

The present invention relates one or more antibodies, particularly nanobodies or VHH single domain antibodies, directed against one or more Campylobacter bacteria targets with roles in Campylobacter colonization, particularly Campylobacter jejuni, in animals, particularly in poultry. The nanobodies are useful in reducing or blocking Campylobacter colonization or infection. The invention relates to methods for reducing or blocking Campylobacter colonization or infection, improving food safety, and reducing bacterial gastroenteritis in animals, including humans.

### BACKGROUND OF THE INVENTION

Campylobacter bacteria remain a major public health concern. Campylobacter are the leading cause of bacterial gastroenteritis in the world. There are 31 different species, with C. jejuni being the most clinically relevant species responsible for up to 80-90% of Campylobacter infections. Campylobacter are normal or ordinary inhabitants of a variety of food producing animals including poultry and chickens are the primary source of human infections (up to 90% of human infections). Clinical symptoms of Campylobacter infections include watery or bloody diarrhea accompanied by abdominal cramps, nausea, fever, and sometimes vomiting. Although C. jejuni infection is acute and self-limiting, in some patients (1:1000) post infection sequalae can lead to severe neurological disorders such as Guillain-Barré Syndrome. In fact, C. jejuni is responsible for 96 million cases of enteric infection globally each year. In the European Union (EU), C. jejuni is responsible for estimated cases of 9 million with an economic burden of around €2.4 billion each year. In the US—an estimated 1.5 million human infections each year with an economic burden of between $1.3 to 6.8 billion dollars per year.

Thus, it is apparent that, food borne gastroenteritis due to Campylobacter infections in food sources, such as in poultry, remains a major issue. There are no current validated and effective interventions for control of Campylobacter. Vaccination remains a challenge including in that the immune response in animals, including poultry, is not fast and robust enough toe reduce C. jejuni in the short time after hatching when colonization initially occurs and before high colonization is achieved within the first 3-6 weeks of life and before harvest as a food source. Development of a safe an effective vaccine or antiinfection therapy targeted to Campylobacter, including in poultry, is needed. Targeting one or more Campylobacter targets with roles in Campylobacter colonization is proposed to provide an effective intervention. The present invention is directed to this unmet need. Antibodies, particularly nanobodies which are single domain antibodies with high affinity and specificity, directed against one or more targets with key roles in Campylobacter colonization, provide a useful and applicable approach.

The citation of references herein shall not be construed as an admission that such is prior art to the present invention.

### SUMMARY OF THE INVENTION

The present invention provides novel domain antibodies, particularly nanobodies, directed against targets with key roles in Campylobacter colonization.

In embodiments, nanobodies directed against Campylobacter antigens, particularly C. jejuni antigens, selected from one or more of CmeC, CadF, CfrA, CjaD and flagella antigens are provided. In embodiments, combinations of one or more nanobodies directed against one or more of CmeC, CadF, CfrA, CjaD and flagella Campylobacter antigens are provided.

In an embodiment, nanobodies directed against CfrA antigen are provided. Exemplary nanobodies denoted ECM-1G7 and ECM-1C11 are provided. In an embodiment, nanobody denoted ECM-1D6 is also provided. VHH sequences for the antibodies are provided.

Selected nanobody sequences against CfrA are set out in FIG. 2. Sequence of the ECM-1C11 nanobody are as follows:

>CmeC-ECM-IC11
(SEQ ID NO: 66)
EVQLVESGGGLVQPGGSLRLSCAASGFTFSDQPMGWYRQAPGQGREWIA
FISSGSGGTTDYKNSVKGRFTISRDNAKNIVYLQMNSLKPDDTARYYCN
FRIDNNYWGQGTQVTVSSAAASGSLEQKLISEEDLNGAAHHHHHHGAA
(SEQ ID NO: 78)
EVQLVESGGGLVQPGGSLRLSCAASGFTFSDQPMGWYRQAPGQGREWIA
FISSGSGGTTDYKNSVKGRFTISRDNAKNIVYLQMNSLKPDDTARYYCN
FRIDNNYWGQGTQVTVSSAAASGSL

Sequence of the ECM-1G7 nanobody are as follows:

>CmeC-ECM-IG7
(SEQ ID NO: 67)
EVQLVESGGDLVQDGGSLRLSCAASGGDFRNPVTGWYRQAPGKQRELVG
LITSDGRTNYGDSVMGRFTISMDTAKNTMYLQMNSLKPEDSAVYYCAAS
LLYGGLQFSTNIWGQGTQVTVSSAAASGSLEQKLISEEDLNGAAHHHHH
HGAA
(SEQ ID NO: 78)
EVQLVESGGDLVQDGGSLRLSCAASGGDFRNPVTGWYRQAPGKQRELVG
LITSDGRTNYGDSVMGRFTISMDTAKNTMYLQMNSLKPEDSAVYYCAAS
LLYGGLQFSTNIWGQGTQVTVSSAAASGSL

Sequence of the ECM-1D6 nanobody are as follows:

>CmeC-ECM-1D6
(SEQ ID NO: 1)
EVOLVESGGGLVQAGGSLRLSCAASGGTFSRYMMGWFRQAPGKEREFVAS
IARSGGTYFADSVKGRLTATRDDAKNTMHLQMNSLKPEDTAVYYCAAGAG
GSYPALLDFEYLVWGQGTQVTVSSAAASGSLEQKLISEEDLNGAAHHHHH
HGAA
(SEQ ID NO: 80)
EVOLVESGGGLVQAGGSLRLSCAASGGTFSRYMMGWFRQAPGKEREFVAS
IARSGGTYFADSVKGRLTATRDDAKNTMHLQMNSLKPEDTAVYYCAAGAG
GSYPALLDFEYLVWGQGTQVTVSSAAASGSL

In an embodiment, the nanobodies comprise an amino acid sequence having at least 80%, at least 90%, at least 95%, at least 98%, at least 99% amino acid identity to the sequences of nanobodies selected from ECM-1G7, ECM-1C11 and ECM-1D6.

In an embodiment, nanobodies directed against CadF antigen are provided. Exemplary nanobodies denoted ECF-1F10 and ECF-1D10 are provided. VHH sequences for these antibodies are provided.

Selected nanobody sequences against CadF are set out in FIG. 4. Sequence of the ECF-1D10 nanobody are as follows:

>CadF-ECF-ID10
(SEQ ID NO: 68)
EVQLVESGGGLVQPGGSLRLSCAASGFTLDDYAVGWFRQAPGKEREWLS
GISSRDGSTVYADSVKGRFTISRDNAKNTYYLQMNSLKPEDTAVYYCAA
AVGYILTRVKSHYNDWSQGTQVTVSSAAASGSLEQKLISEEDLNGAAHH
HHHHGAA
(SEQ ID NO: 81)
EVQLVESGGGLVQPGGSLRLSCAASGFTLDDYAVGWFRQAPGKEREWLS
GISSRDGSTVYADSVKGRFTISRDNAKNTYYLQMNSLKPEDTAVYYCAA
AVGYILTRVKSHYNDWSQGTQVTVSSAAASGSL

Sequence of the ECF-1F10 nanobody are as follows:

>CadF-ECF-1F10
(SEQ ID NO: 69)
EVQLVESGGGSVQAGGSLRLSCTASIRAGNRYAMGWFRQAPGKEREFVA
GISWSGGNTYHADSVNGRFTISRDNAKNTLYLTMNSLKSEDTAVYYCAA
SWKPLTFRGDDYTYWGQGTQVTVSSAAASGSLEQKLISEEDLNGAAHHH
HHHGAA
(SEQ ID NO: 82)
EVQLVESGGGSVQAGGSLRLSCTASIRAGNRYAMGWFRQAPGKEREFVA
GISWSGGNTYHADSVNGRFTISRDNAKNTLYLTMNSLKSEDTAVYYCAA
SWKPLTFRGDDYTYWGQGTQVTVSSAAASGSL

In an embodiment, the nanobodies comprise an amino acid sequence having at least 80%, at least 90%, at least 95%, at least 98%, at least 99% amino acid identity to the sequences of nanobodies selected from ECF-1F10 and ECF-1D10.

In an embodiment, nanobodies directed against CfrA antigen are provided. Exemplary nanobodies denoted ECA-1G7 are provided. VHH sequences for this antibodies are provided.

Selected nanobody sequences against CfrA are set out in FIG. 6. Sequence of the ECA-1G7 nanobody are as follows:

>CfrA-ECA-1G7
(SEQ ID NO: 29)
EVQLVESGGGLVQPGGSLRLSCTASGSSLDYYAIGWFRQAPGKEREGVS
CISSRDGRIYWADSVEGRFTISRDNGKNTVYLQMNSLKPEDTAVYYCAT
SRWSFCPSDWSPVPSPFGSWGQGTQVTVSSAAASGSLEQKLISEEDLNG
AAHHHHHHGAA
(SEQ ID NO: 83)
EVQLVESGGGLVQPGGSLRLSCTASGSSLDYYAIGWFRQAPGKEREGVS
CISSRDGRIYWADSVEGRFTISRDNGKNTVYLQMNSLKPEDTAVYYCAT
SRWSFCPSDWSPVPSPFGSWGQGTQVTVSSAAASGSL

In an embodiment, the nanobodies comprise an amino acid sequence having at least 80%, at least 90%, at least 95%, at least 98%, at least 99% amino acid identity to the sequences of nanobody ECA-1G7.

In an embodiment, nanobodies directed against CjaD antigen are provided. Exemplary nanobodies denoted ECD-1D3 and ECD-1A7 are provided. VHH sequences for these antibodies are provided.

Selected nanobody sequences against CjaD are set out in FIG. 8.

Sequence of the ECD-1D3 nanobody are as follows:

>CjaD-ECD-1D3
(SEQ ID NO: 84)
EVQLVESGGGLVQTGGSLRLSCATSGFTFEYSAIAWFRQAPGKGREGVS
CISNRDGTTVYADSVKGRFTISSDNAKNTVYLQMNSLTPEDTGVYYCAA
DAGVYTADYCRDSRYDPVSKDAWGKGTLVTVSSAAASGSLEQKLISEED
LNGAAHHHHHHGAA
(SEQ ID NO: 49)
EVQLVESGGGLVQTGGSLRLSCATSGFTFEYSAIAWFRQAPGKGREGVS
CISNRDGTTVYADSVKGRFTISSDNAKNTVYLQMNSLTPEDTGVYYCAA
DAGVYTADYCRDSRYDPVSKDAWGKGTLVTVSSAAASGSL

Sequence of the ECD-1A7 nanobody are as follows:

>CjaD-ECD-1A7
(SEQ ID NO: 53)
EVQLVESGGGLVQAGGSLRLSCATSGFTPGDYAIGWFRQAPGKAREGVS
CLSTRDGTTYYADSVKGRFTISTDNAQNTVYLQMNSLKPEDTAVYYCAG
EQISFRAVYYCTEYEPVYWSQGTQVTVSSAAASGSLEQKLISEEDLNGA
AHHHHHHGAA
(SEQ ID NO: 85)
EVQLVESGGGLVQAGGSLRLSCATSGFTPGDYAIGWFRQAPGKAREGVS
CLSTRDGTTYYADSVKGRFTISTDNAQNTVYLQMNSLKPEDTAVYYCAG
EQISFRAVYYCTEYEPVYWSQGTQVTVSSAAASGSL

In an embodiment, the nanobodies comprise an amino acid sequence having at least 80%, at least 90%, at least 95%, at least 98%, at least 99% amino acid identity to the sequences of nanobodies selected from ECD-1D3 and ECD-1A7.

In an embodiment, nanobodies directed against C. jejuni flagella as antigen are provided. Exemplary nanobodies denoted ECF-1C4, ECF-1F4, ECF-1B6, ECF1G8 and ECF-1D9 are provided. VHH sequences for these antibodies are provided.

Selected nanobody sequences against C. jejuni flagella are set out in FIG. 11. Sequence of the ECF-1C4 nanobody are as follows:

>Flagella-ECF-1C4
(SEQ ID NO: 73)
EVQLVESGGGLVQAGGSLRVSCAASGRAVHNYALNWFRQAPGKEREFVA
AISWTRRTYYANSVRGRFTISRDNNGNMVHLQMSNLKSEDTALYYCAAA
TTWPRLDGAEYWGQGTQVTVSSAAADYKDDDDKGAAHHHHHGAA
(SEQ ID NO: 86)
EVQLVESGGGLVQAGGSLRVSCAASGRAVHNYALNWFRQAPGKEREFVA
AISWTRRTYYANSVRGRFTISRDNNGNMVHLQMSNLKSEDTALYYCAAA
TTWPRLDGAEYWGQGTQVTVSS

Sequence of the ECF-1F4 nanobody are as follows:

>Flagella-ECF-1F4
(SEQ ID NO: 74)
EVQLVESGGGLVQPGGSLRLSCEAAGSILRVNSMGWYRQAPGKQRDLVA
IITSNNDAKYSDSVKGRFTISRDNAKNTVYLQMSSLKPDDTAVYYCNAY
VETAGWIPTTHNLWGQGTQVTVSSAAADYKDDDDKGAAHHHHHHGAA
(SEQ ID NO: 87)
EVQLVESGGGLVQPGGSLRLSCEAAGSILRVNSMGWYRQAPGKQRDLVA
IITSNNDAKYSDSVKGRFTISRDNAKNTVYLQMSSLKPDDTAVYYCNAY
VETAGWIPTTHNLWGQGTQVTVSSAAA

Sequence of the ECF-1B6 nanobody are as follows:

>Flagella-ECF-1B6
(SEQ ID NO: 75)
EVQLVESGGGLVQSGGSLRLSCAASGRIFSITNMGWYRQAPGEQRELVA
DMPSGGSTNYADSVKGRFTISRDIAKNTVYLQMNSLKPEDTAVYYCNLK
MSQPGWLVTNHNFWGQGTQVTVSSAAADYKDDDDKGAAHHHHHHGAA
(SEQ ID NO: 88)
EVQLVESGGGLVQSGGSLRLSCAASGRIFSITNMGWYRQAPGEQRELVA
DMPSGGSTNYADSVKGRFTISRDIAKNTVYLQMNSLKPEDTAVYYCNLK
MSQPGWLVTNHNFWGQGTQVTVSSAAA

Sequence of the ECF-1G8 nanobody are as follows:

>Flagella-ECF-1G8
(SEQ ID NO: 76)
EVQLVESGGGLVEAGGSLTLSCTTSEPTSLLNLMGWWRQGPGKEREFVA
SSNWSGKLVDYADGVEGRFTVIRNEDENAISLQMNSLTVEDTAIYYCAA
SRLGLPRSAQAYQYWGQGTQVTVSSAAADYKDDDDKGAAHHHHHHGAA
(SEQ ID NO: 89)
EVQLVESGGGLVEAGGSLTLSCTTSEPTSLLNLMGWWRQGPGKEREFVA
SSNWSGKLVDYADGVEGRFTVIRNEDENAISLQMNSLTVEDTAIYYCAA
SRLGLPRSAQAYQYWGQGTQVTVSSAAA

Sequence of the ECF-1D9 nanobody are as follows:

>Flagella-ECF-1D9
(SEQ ID NO: 77)
EVQLVESGGGLVQPGGSLRLSCATSGFAFDNYCMYWVRQAPGKGLEWIS
GITNGGSFSYYADSVKGRFTISRDNAKNTLFLEMNSLKSDDTAVYYCAI
HGHGCTWDSLRTTSGPRYRGQGTQVTVSSAAADYKDDDDKGAAHHHHHH
GAA
(SEQ ID NO: 90)
EVQLVESGGGLVQPGGSLRLSCATSGFAFDNYCMYWVRQAPGKGLEWIS
GITNGGSFSYYADSVKGRFTISRDNAKNTLFLEMNSLKSDDTAVYYCAI
HGHGCTWDSLRTTSGPRYRGQGTQVTVSSAAA

In an embodiment, the nanobodies comprise an amino acid sequence having at least 80%, at least 90%, at least 95%, at least 98%, at least 99% amino acid identity to the sequences of nanobodies selected from ECF-1C4, ECF-1F4, ECF-1B6, ECF1G8 and ECF-1D9.

The underlined regions in the above nanobody amino acid sequences indicate tag sequences utilized in generating and isolating the sequences. The tag sequences can be useful in assays and in diagnostics, etc. The antibodies have cMyc-His tags or FLAG-His tags (flagellar antibodies have the FLAG-His tags all the others have cMyc-His tags). In embodiments, the nanobodies include the tag sequences. In embodiments, the nanobodies do not include the tag sequences. Nanobody amino acid sequences with and without the tags are presented and provided herein and above.

In embodiments, combinations of one or more nanobodies directed against one or more of CmeC, CadF, CfrA, CjaD and flagella Campylobacter antigens are provided, particularly combinations of the antibodies selected and provided above. In an embodiment, the invention provides and relates to combinations of two or more of CmeC, CadF, CfrA, CjaD and flagella Campylobacter antigens are provided, particularly combinations of the antibodies selected and provided above.

In an embodiment, a combination of antibodies directed to each of C. jejuni antigens CmeC, CadF, CfrA, CjaD and flagella are provided, particularly combinations of the antibodies selected and provided above, each antibody in combination directed to one of the relevant and target antigens.

In embodiments, the antibodies directed individually to each of C. jejuni antigens CmeC, CadF, CfrA, CjaD and flagella are capable of binding to their specific target protein or flagella and neutralizing or inhibiting the activity of their target or in the instance of flagella in blocking or inhibiting flagellar movement or function.

In one embodiment, flagellar nanobodies were generated using native flagella, which is glycosylated. In another embodiment, multimeric nanobodies are generated to further enhance the potency/in vivo efficacy. Multiple copies of nanobodies are combined using a GS or similar linker where the number of copies can vary from 2 to 5.

In an embodiment, the nanobody of the invention comprises a heavy chain variable region VHH sequence as set out herein, including in FIG. 2, 4, 6, 8, 10 or 11. In an embodiment, the nanobody comprises an amino acid sequence having at least 80%, at least 90%, at least 95%, at least 98%, at least 99% amino acid identity to a VHH sequence as set out herein, including in FIG. 2, 4, 6, 8, 10 or 11. Any such nanobody is capable of binding specifically to the applicable antigen, particularly in each instance the applicable CmeC, CadF, CfrA, CjaD or flagella Campylobacter proteins.

The invention provides antibodies specifically directed against CmeC, CadF, CfrA, CjaD and flagella Campylobacter antigens for diagnostic and therapeutic purposes. In particular, antibodies specific for each of CmeC, CadF, CfrA, CjaD and flagella Campylobacter antigens are provided, wherein said antibodies recognize and are capable of binding CmeC, CadF, CfrA, CjaD and flagella Campylobacter antigens.

The antibodies of the present invention have diagnostic and therapeutic use in Campylobacter infections and colonization, including modulating the immune response of an animal to Campylobacter and modulating the infection and colonization of Campylobacter in an animal. The antibodies of the invention are applicable in characterizing and in modulating the activity of CmeC, CadF, CfrA, CjaD and flagella Campylobacter proteins. The antibodies of the invention are applicable in modulating the activity of CmeC, CadF, CfrA, CjaD and flagella Campylobacter proteins and acting as a prophylactic or therapeutic to prevent or inhibit Campylobacter, including Campylobacter jejuni infection and/or colonization.

The antibodies of the present invention have diagnostic and therapeutic use in Campylobacter infections and colonization, including modulating the immune response of an animal to Campylobacter and modulating the infection and colonization of Campylobacter in an animal. The antibodies of the invention are applicable in characterizing and in modulating the activity of one or more or any of CmeC, CadF, CfrA, CjaD and flagella Campylobacter proteins, The antibodies of the invention are applicable in modulating the activity of one or more or any of CmeC, CadF, CfrA, CjaD and flagella Campylobacter proteins and acting as a prophylactic or therapeutic to prevent or inhibit Campylobacter, including Campylobacter jejuni infection and/or colonization.

The antibodies of the present invention have diagnostic and therapeutic use in bacterial gastroenteritis in animals, including humans, that ingest food, such as chicken, from a Campylobacter infected or colonized animal. The antibodies of the invention are applicable in characterizing and in modulating the activity of one or more Campylobacter proteins and thereby reducing or alleviating bacterial enteritis. The antibodies of the invention are applicable in characterizing and in modulating the activity of one or more of or any CmeC, CadF, CfrA, CjaD and flagella Campylobacter proteins. The antibodies of the invention are applicable in modulating the activity of one or more or any CmeC, CadF, CfrA, CjaD and flagella Campylobacter proteins and acting as a prophylactic or therapeutic to prevent or alleviate bacterial gastroenteritis in animals, including humans, that ingest food, such as chicken, from a Campylobacter infected or colonized animal.

Methods are provided for identifying or characterizing Campylobacter, such as in an animal infected or colonized with Campylobacter utilizing one or more of the nanobodies provided herein and directed against CmeC, CadF, CfrA, CjaD or flagella Campylobacter proteins.

Methods are provided for inhibiting Campylobacter bacteria, such as in an animal infected or colonized with Campylobacter, utilizing one or more of the nanobodies provided herein and directed against CmeC, CadF, CfrA, CjaD or flagella Campylobacter proteins. Methods are provided for inhibiting Campylobacter bacteria, such as in an animal infected or colonized with Campylobacter, comprising administering to the animal one or more of the nanobodies provided herein and directed against CmeC, CadF, CfrA, CjaD or flagella Campylobacter proteins.

Methods are provided for blocking or reducing colonization by Campylobacter in an animal, such as in an animal infected or colonized with Campylobacter jejuni, utilizing one or more of the nanobodies provided herein and directed against CmeC, CadF, CfrA, CjaD or flagella Campylobacter proteins. Methods are provided for blocking or reducing colonization by Campylobacter in an animal, such as in an animal infected or colonized with Campylobacter jejuni, comprising administering to the animal one or more of the nanobodies provided herein and directed against CmeC, CadF, CfrA, CjaD or flagella Campylobacter proteins.

In further embodiments, the invention provides an isolated nucleic acid which comprises a sequence encoding a VHH polypeptide described herein, particularly a nanobody provided herein and directed against CmeC, CadF, CfrA, CjaD or flagella Campylobacter protein. In an embodiment, the invention includes nucleic acid encoding one or more nanobody, including nanobody amino acid sequence disclosed and described herein, or set out in FIG. 2, 4, 6, 8, 10 or 11.

The present invention also relates to a recombinant DNA molecule or cloned gene, or a degenerate variant thereof, which encodes an antibody of the present invention; preferably a nucleic acid molecule, in particular a recombinant DNA molecule or cloned gene, encoding the antibody VH, particularly the CDR region sequences, which is capable of encoding a heavy chain sequence described and as set out herein, including as set out on FIG. 2, 4, 6, 8, 10 or 11.

In accordance with the invention, methods for treatment, alleviation or modulation of Campylobacter bacteria, or infection or colonization due to Camylobacter, comprising administering the antibodies of the invention or pharmaceutical compositions thereof are provided herein.

Thus, in an embodiment of the invention the nanobodies may be administered alone or in combination with other treatments, therapeutics or agents, either simultaneously or sequentially dependent upon the condition to be treated.

The invention includes compositions and or kits, comprising one or more nanobodies of the invention together with one or more immunomodulatory or immunogenic or antibacterial proteins or peptides. The compositions include pharmaceutical compositions and immunological compositions. The nanobodies or compositions of the invention may be administered systemically or in a targeted fashion, including administration to an affected organ or organ of interest, such as to the gastrointestinal tract.

The nanobodies of the present invention, and in a particular embodiment one or more nanobody having sequence as set out herein or as represented in FIG. 2, 4, 6, 8, 10 or 11, or active fragments thereof, and recombinant or synthetic nanobodies derived therefrom, particularly comprising the heavy chain CDR region sequences of the nanobodies can be prepared in pharmaceutical compositions, including a suitable vehicle, carrier or diluent, or including an adjuvant and/or immune modulator, for administration. Such pharmaceutical compositions may also include means for modulating the half-life of the nanobodies or fragments by methods known in the art such as pegylation.

Pharmaceutical compositions or immunogenic compositions of the invention may further comprise additional antibodies or therapeutic agents. In an aspect, such other agents or therapeutics may be selected from anti-bacterial agents or immune modulators or anti-inflammatory agents. Pharmaceutical compositions or immunological compositions may comprise a combination of one or more, two or more, three or more or four or more or five unique nanobodies as set out and provided herein. Compostions may comprise a combination of nanobodies directed against CmeC, CadF, CfrA, CjaD and flagella Campylobacter proteins, particularly a combination comprising each of a nanobody described herein specific for Campylobacter CmeC, CadF, CfrA, CjaD flagella protein. Various such combinations are contemplated herein.

The diagnostic utility of the present invention extends to the use of the nanobodies of the present invention in assays to characterize cellular samples or to screen for Campylobacter or Campylobacter infection or colonization, including in vitro and in vivo diagnostic assays. Nanobodies of the invention may carry a detectable or functional label. The specific binding members may carry a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²¹I, ¹²⁴I, ¹²⁵I, ¹³¹I, ¹¹¹In, ¹¹⁷Lu, ²¹¹At, ¹⁹⁸Au, ⁶⁷Cu, ²²⁵Ac, ²¹³Bi, ⁹⁹Tc and ¹⁸⁶Re. In an aspect, the label may be an enzyme, including wherein detection may be accomplished by any of the presently utilized colorimetric, spectrophotometric, fluorospectrophotometric, amperometric or gasometric techniques known in the art.

Immunoconjugates or antibody fusion proteins of the present invention, wherein nanobodies of the present invention are conjugated or attached to other molecules or agents further include, but are not limited to nanobody(ies) conjugated to a immunomodulator, cytokine, cytotoxic agent, antibacterial agent, antibiotic or drug.

Other objects and advantages will become apparent to those skilled in the art from a review of the ensuing detailed description, which proceeds with reference to the following illustrative drawings, and the attendant claims.

### BRIEF DESCRIPTION OF THE DRAWINGS

FIG. 1 provides a depiction and overview of sources, transmissions and outcomes of Campylobacter jejuni infection.

FIG. 2 provides alignment of the sequences of the anti-CmeC VHHs picked from master plate ECM-1 (SEQ ID NOS: 1-12, respectively top to bottom). This shows the sequence alignment of the clones that were picked from the selection outputs. There is a diversity of around 8 different VHH sequences, which were derived from two families (KEREF and KQREL) within the sequences.

FIG. 3 depicts dose response binding of the selected VHH to CmeC.

FIG. 4 provides ligament of the sequences of the VHH to CadF picked from master plate ECF-1 (SEQ ID NOS: 13-27, respectively top to bottom). FIG. 4 shows the sequence alignment of the clones that were picked from the selection outputs. There is a diversity of around 9 different VHH sequences based on the epitope families, which were derived from two different germline families (KEREF and KGLEW) within the sequences.

FIG. 5 depicts dose response binding of the selected VHH to CadF.

FIG. 6 provides alignment of the sequences of the VHH to CfrA picked from master plate ECA-1 (SEQ ID NOS: 28-39, respectively top to bottom). FIG. 6 shows the sequence alignment of the clones that were picked from the selection outputs. There is a diversity of around 6 different VHH sequences based on the epitope families, which were derived from three different germline families (KEREF, KQREL and KEREG) within the sequences.

FIG. 7 depicts dose response binding of the selected VHH to CfrA.

FIG. 8 provides alignment of the sequences of the VHHs to CjaD picked from master plate ECD-1 (SEQ ID NOS: 40-54, respectively top to bottom). FIG. 8 shows the sequence alignment of the clones that were picked from the selection outputs. There is a diversity of around 12 different VHH sequences based on the epitope families, which were derived from three different germline families (KEREF, KQREL and KGLEW) within the sequences.

FIG. 9 depicts Dose response binding of the selected VHH to CjaD.

FIG. 10 provides an alignment of amino acid sequences of the master plate antibodies to determine the diversity of the VHH binding to Cj flagella in peri-ELISA provides Amino acid sequences of Cj flagella-binding VHH in pMEK222.

FIG. 11 depicts amino acid sequences of Cj flagella-binding VHH. These VHH sequences have an N terminal FLAG-His tag sequence (SEQ ID NOS: 55-64, respectively top to bottom).

FIG. 12 provides binding of purified VHH to immobilized Cj flagella. Bound VHH were detected using rabbit-anti-VHH (Cat #QE19), followed by donkey-anti-rabbit HRP and OPD as substrate.

FIG. 13 provides a sequence alignment and comparison of selected nanobodies against distinct Campylobacter protein targets (SEQ ID NOS: 65-77, respectively top to bottom). A consensus nanobody sequence is provided. The degree of identity between the sequences for each amino acid and consensus amino acid is depicted in a bar graphical presentation. This comparison points to regions of distinct sequence, which provides one skilled in the art direction as to the applicable heavy chain VHH CDRs and CDR1, CDR2, and CDR3 regions and sequences for each nanobody. Sequences of selected nanobodies ECM-1C11, ECM-1G7, ECF-1F10, ECF-1D10, ECA-1G7, ECD-1D3, ECD-1A7, ECF-1C4, ECF-1F4, ECF-1B6, ECF-1G8 and ECF-1D9 are compared.

FIG. 14 Mean log 10 CFU counts of Campylobacter from cloacal swabs in groups treated with different anti-Campylobacter vaccine candidates.

FIG. 15A provides effect of anti-flagellar nanobodies against US C. jejuni isolates.

FIG. 15B provides effect of anti-flagellar nanobodies against US C. jejuni isolates

FIG. 16A provides effect of anti-flagellar nanobodies against EU C. jejuni isolates.

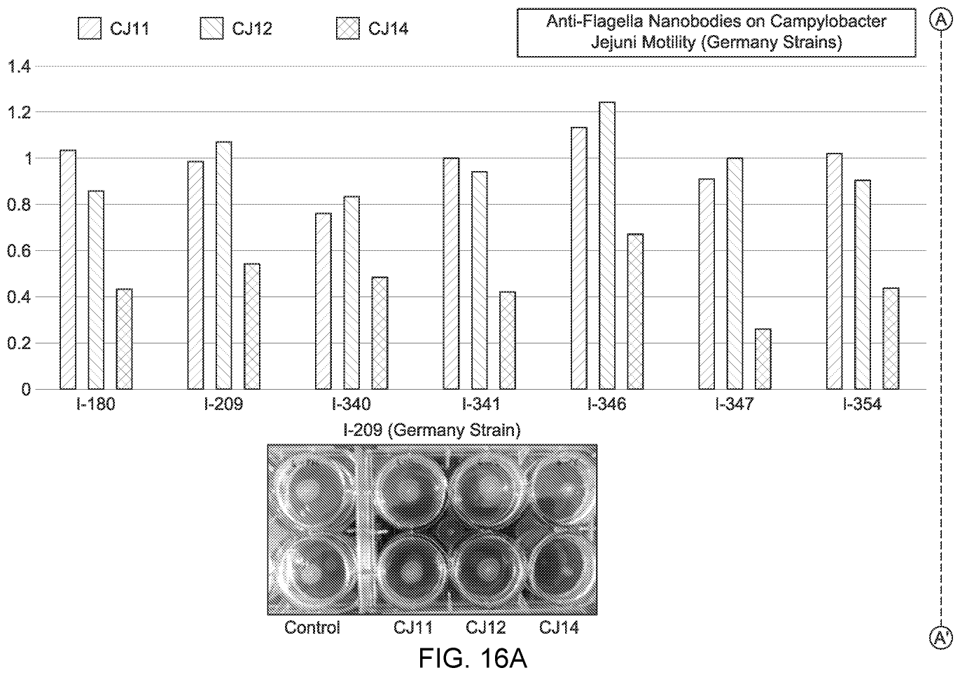

FIG. 16B provides effect of anti-flagellar nanobodies against EU C. jejuni isolates.

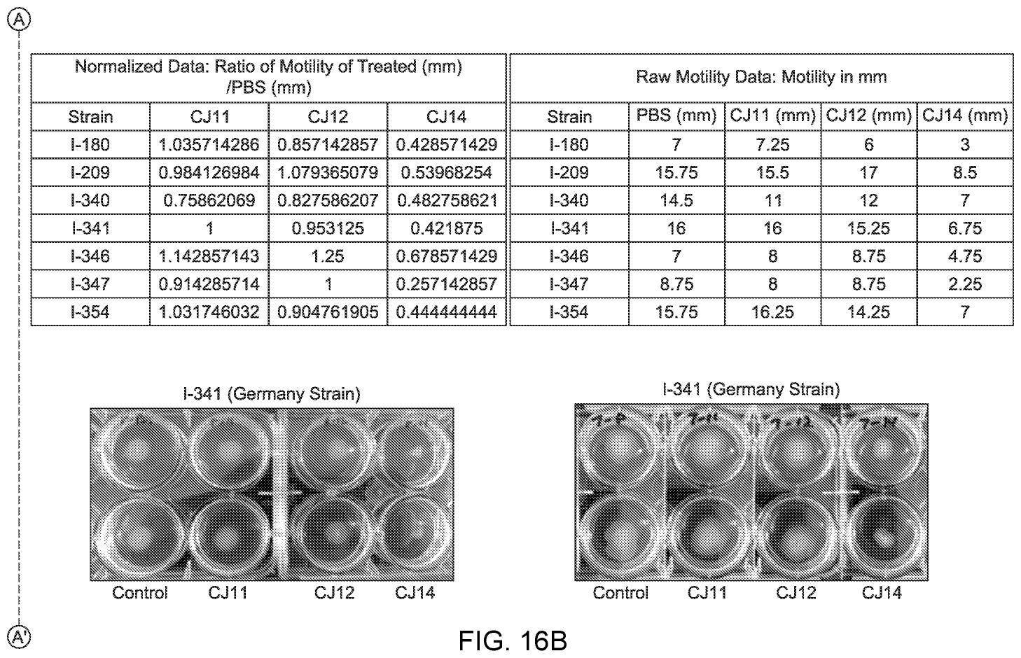

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. Higgins eds. (1985)]; “Transcription And Translation” [B. D. Hames & S. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

As used herein, the terms “colonize” and “colonization” include “temporarily colonize” and “temporary colonization”.

As used herein, “carrier”, “acceptable carrier”, or “pharmaceutical carrier” are used interchangeably and refer to a diluent, adjuvant, excipient, or vehicle with which the compound is administered. Such carriers can be sterile liquids, such as water and oils, including those of petroleum, animal, vegetable, or synthetic origin; such as peanut oil, soybean oil, mineral oil, sesame oil, and the like. Water or aqueous solution saline solutions and aqueous dextrose and glycerol solutions are preferably employed as carriers, in some embodiments as injectable solutions. Alternatively, the carrier can be a solid dosage form carrier, including but not limited to one or more of a binder (for compressed pills), a glidant, an encapsulating agent, a flavorant, and a colorant. The choice of carrier can be selected with regard to the intended route of administration and standard pharmaceutical practice. See Handbook of Pharmaceutical Excipients, (Sheskey, Cook, and Cable) 2017, 8th edition, Pharmaceutical Press; Remington's Pharmaceutical Sciences, (Remington and Gennaro) 1990, 18th edition, Mack Publishing Company; Development and Formulation of Veterinary Dosage Forms (Hardee and Baggot), 1998, 2nd edition, CRC Press.

As used herein, “delivery” or “administration” means the act of providing a beneficial activity to a host. The delivery may be direct or indirect. An administration could be by an oral, nasal, or mucosal route. For example without limitation, an oral route may be an administration through drinking water, a nasal route of administration may be through a spray or vapor, and a mucosal route of administration may be through direct contact with mucosal tissue. Mucosal tissue is a membrane rich in mucous glands such as those that line the inside surface of the nose, mouth, esophagus, trachea, lungs, stomach, gut, intestines, and anus. In the case of birds, administration may be in ovo, i.e. administration to a fertilized egg. In ovo administration can be via a liquid which is sprayed onto the egg shell surface, or an injected through the shell.

As used herein, the terms “treating”, “to treat”, or “treatment”, include restraining, slowing, stopping, inhibiting, reducing, ameliorating, or reversing the progression or severity of an existing symptom, disorder, condition, or disease. A treatment may also be applied prophylactically to prevent or reduce the incidence, occurrence, risk, or severity of a clinical symptom, disorder, condition, or disease.

As used herein, “animal” includes bird, poultry, a human, or a non-human mammal. Specific examples include chickens, turkey, dogs, cats, cattle, salmon, fish, swine and horse. The chicken may be a broiler chicken, egg-laying, or egg-producing chicken. As used herein, the term “poultry” includes domestic fowl, such as chickens, turkeys, ducks, and geese.

In embodiments, animal includes and refers particularly to an animal susceptible to Campylobacter infection. In embodiments, animal includes and refers particularly to an animal susceptible to bacterial gastroenteritis due to Campylobacter bacteria or infection.

As used herein, “gut” refers to the gastrointestinal tract including stomach, small intestine, and large intestine. The term “gut” may be used interchangeably with “gastrointestinal tract”.

As used herein, “subject” includes bird, poultry, a human, or a non-human animal. Specific examples include chickens, turkey, dogs, cats, cattle, and swine. The chicken may be a broiler chicken, egg-laying or egg-producing chicken. As used herein, the term “poultry” includes domestic fowl, such as chickens, turkeys, ducks, quail, and geese.

The term “antibody” describes an immunoglobulin whether natural or partly or wholly synthetically produced. The term also covers any polypeptide or protein having a binding domain which is, or is homologous to, an antibody binding domain. An “antibody” is any immunoglobulin, including antibodies and fragments thereof, that binds a specific epitope. The term encompasses polyclonal, monoclonal, and chimeric antibodies. The term “antibody(ies)” includes a wild type immunoglobulin (Ig) molecule, generally comprising four full length polypeptide chains, two heavy (H) chains and two light (L) chains.

The term antibody includes and encompasses antibody fragments and domain antibodies. Antibody includes a molecule comprising at least one polypeptide chain that is not full length, including (i) a Fab fragment, which is a monovalent fragment consisting of the variable light (VL), variable heavy (VH), constant light (CL) and constant heavy 1 (CH1) domains; (ii) a F(ab′)2 fragment, which is a bivalent fragment comprising two Fab fragments linked by a disulfide bridge at the hinge region; (iii) a heavy chain portion of an Fab (Fd) fragment, which consists of the VH and CH1 domains; (iv) a variable fragment (Fv), which consists of the VL and VH domains of a single arm of an antibody, (v) a domain antibody (dAb) fragment, which comprises a single variable domain (Ward, E. S. et al., Nature 341, 544-546 (1989)); (vi) a camelid antibody or nanobody; (vii) an isolated complementarity determining region (CDR); (viii) a Single Chain Fv Fragment wherein a VH domain and a VL domain are linked by a peptide linker which allows the two domains to associate to form an antigen binding site (Bird et al, Science, 242, 423-426, 1988; Huston et al, PNAS USA, 85, 5879-5883, 1988); (ix) a diabody, which is a bivalent, bispecific antibody in which VH and/or VL domains are expressed on a single polypeptide chain, but using a linker that is too short to allow for pairing between the two domains on the same chain, thereby forcing the domains to pair with the complementarity domains of another chain and creating two antigen binding sites (WO94/13804; P. Holliger et al Proc. Natl. Acad. Sci. USA 90 6444-6448, (1993)); and (x) a linear antibody, which comprises a pair of tandem Fv segments (VH-CH1-VH-CH1) which, together with complementarity light chain polypeptides, form a pair of antigen binding regions; (xi) multivalent antibody fragments (scFv dimers, trimers and/or tetramers (Power and Hudson, J Immunol. Methods 242:193-204 9 (2000)); (xii) a minibody, which is a bivalent molecule comprised of scFv fused to constant immunoglobulin domains, CH3 or CH4, wherein the constant CH3 or CH4 domains serve as dimerization domains (Olafsen T et al (2004) Prot Eng Des Sel 17 (4): 315-323; Hollinger P and Hudson P J (2005) Nature Biotech 23 (9): 1126-1136); and (xiii) other non-full length portions of heavy and/or light chains, or mutants, variants, or derivatives thereof, alone or in any combination.

Antibody(ies) comprising linked nanobodies, such as multimeric and bi-specific versions are included in embodiments of the invention. Thus, two or more nanobodies or sequences encoding two or more nanobodies can be covalently linked, through a linker sequence or any such other recognized and applicable means, to form a bispecific or multimeric form of the nanobody(ies). In an embodiment, two distinct nanobodies are linked. In an embodiment a single nanobody is mutltimerized through linkage, which may have applicability to increase binding, avidity, affinity. In an embodiment, two or more unwue nanobodies, including nanobodies directed against distinct Campylobacter protein targets are linked.

The term “antigen binding domain” describes the part of an antibody which comprises the area which specifically binds to and is complementary to part or all of an antigen. Where an antigen is large, an antibody may bind to a particular part of the antigen only, which part is termed an epitope. An antigen binding domain may be provided by one or more antibody variable domains.

The term “adjuvant(s)” describes a substance, compound, agent or material useful for improving an immune response or immune cell or component stimulation, and may in some instances be combined with any particular antigen in an immunological, pharmaceutical or vaccine composition. Adjuvants can be used to increase the amount of antibody and effector T cells produced and to reduce the quantity of antigen or immune stimulant or modulator and the frequency of injection. Although some antigens are administered without an adjuvant, there are many antigens that lack sufficient immunogenicity to stimulate a useful immune response in the absence of an effective adjuvant. Adjuvants also improve the immune response from “self-sufficient” antigens, in that the immune response obtained may be increased or the amount of antigen administered may be reduced. An adjuvant can serve as a tissue depot that slowly releases the antigen and also as a lymphoid system activator that non-specifically enhances the immune response (Hood et al., Immunology, Second Ed., 1984, Benjamin/Cummings: Menlo Park, California, p. 384). In a preferred aspect an adjuvant is physiologically and/or pharmaceutically acceptable in a mammal, particularly a human. The standard adjuvant for use in laboratory animals is Freund's adjuvant. Freund's Complete adjuvant (FCA) is an emulsion containing mineral oil and killed mycobacteria in saline. Freund's incomplete adjuvant (FIA) omits the mycobacteria. Both FIA and FCA induce good humoral (antibody) immunity, and FCA additionally induces high levels of cell-mediated immunity. However, neither FCA nor FIA are acceptable for clinical use due to the side effects. In particular, mineral oil is known to cause granulomas and abscesses, and Mycobacterium tuberculosis is the agent responsible for tuberculosis. Previously known and utilized adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, and potentially useful human adjuvant such as BCG (bacille Calmette-Guerin) and Corynebacterium parvum. Mineral salt adjuvants include but are not limited to: aluminum hydroxide, aluminum phosphate, calcium phosphate, zinc hydroxide and calcium hydroxide. Preferably, the adjuvant composition further comprises a lipid of fat emulsion comprising about 10% (by weight) vegetable oil and about 1-2% (by weight) phospholipids. Preferably, the adjuvant composition further optionally comprises an emulsion form having oily particles dispersed in a continuous aqueous phase, having an emulsion forming polyol in an amount of from about 0.2% (by weight) to about 49% (by weight), optionally a metabolizable oil in an emulsion-forming amount of up to 15% (by weight), and optionally a glycol ether-based surfactant in an emulsion-stabilizing amount of up to about 5% (by weight). There have been many substances that have been tried to be used as adjuvants, such as the lipid-A portion of gram negative bacterial endotoxin, and trehalose dimycolate of mycobacteria. The phospholipid lysolecithin exhibited adjuvant activity (Amold et al., Eur. J Immunol. 9:363-366, 1979). Some synthetic surfactants exhibited adjuvant activity, including dimethyldioctadecyl ammonium bromide (DDA) and certain linear polyoxypropylenepolyoxyethylene (POP-POE) block polymers (Snippe et al., Int. Arch. Allergy Appl. Immunol. 65:390-398, 1981; and Hunter et al., J. Immunol. 127:1244-1250, 1981).

The term “specific” may be used to refer to the situation in which one member of a specific binding pair will not show any significant binding to molecules other than its specific binding partner(s). The term is also applicable where e.g. an antigen binding domain is specific for a particular epitope which is carried by a number of antigens, in which case the specific binding member carrying the antigen binding domain will be able to bind to the various antigens carrying the epitope.

The term “comprise” generally used in the sense of include, that is to say permitting the presence of one or more features or components.

The term “consisting essentially of” refers to a product, particularly a peptide sequence, of a defined number of residues which is not covalently attached to a larger product. In the case of the peptide of the invention referred to above, those of skill in the art will appreciate that minor modifications to the N- or C-terminal of the peptide may however be contemplated, such as the chemical modification of the terminal to add a protecting group or the like, e.g. the amidation of the C-terminus.

The amino acid residues described herein are preferred to be in the “L” isomeric form. However, residues in the “D” isomeric form can be substituted for any L-amino acid residue, as long as the desired functional property of immunoglobulin-binding is retained by the polypeptide. NH₂ refers to the free amino group present at the amino terminus of a polypeptide. COOH refers to the free carboxy group present at the carboxy terminus of a polypeptide. Single letter abbreviations for amino acid residues are known in the art and one skilled in the art will recognize the amino acid each and any single letter refers to.

It should be noted that all amino-acid residue sequences are represented herein by formulae whose left and right orientation is in the conventional direction of amino-terminus to carboxy-terminus.

A “replicon” is any genetic element (e.g., plasmid, chromosome, virus) that functions as an autonomous unit of DNA replication in vivo; i.e., capable of replication under its own control.

A “vector” is a replicon, such as plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

The term “oligonucleotide,” as used herein in referring to the probe of the present invention, is defined as a molecule comprised of two or more ribonucleotides, preferably more than three. Its exact size will depend upon many factors which, in turn, depend upon the ultimate function and use of the oligonucleotide.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

As used herein, the terms “restriction endonucleases” and “restriction enzymes” refer to bacterial enzymes, each of which cut double-stranded DNA at or near a specific nucleotide sequence.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art.

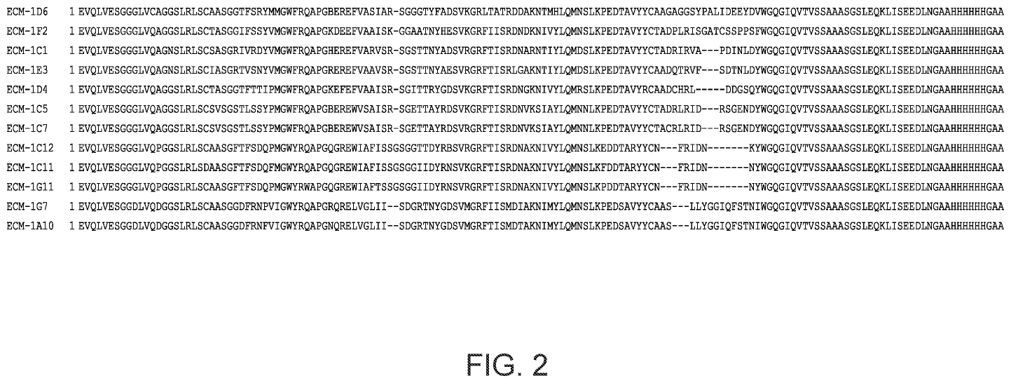

It should be appreciated that also within the scope of the present invention are DNA sequences encoding nanobodies of the invention which code for e.g. an antibody having amino acid sequence as provided herein and/or as described in FIG. 2, 4, 6, 8, 10 or 11, or comprising the CDR domain region sequences set out herein or in FIG. 2, 4, 6, 8, 10 or 11, but which are degenerate thereto. By “degenerate to” is meant that a different three-letter codon is used to specify a particular amino acid. It is well known in the art the codons that can be used interchangeably to code for each specific amino acid.

Mutations can be made in the sequences encoding the amino acids, nanobodies, CDR region sequences thereof including those sequences set out in FIG. 2, 4, 6, 8, 10 or 11, such that a particular codon is changed to a codon which codes for a different amino acid. Such a mutation is generally made by making the fewest nucleotide changes possible. A substitution mutation of this sort can be made to change an amino acid in the resulting protein in a non-conservative manner (for example, by changing the codon from an amino acid belonging to a grouping of amino acids having a particular size or characteristic to an amino acid belonging to another grouping) or in a conservative manner (for example, by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present invention includes sequences containing amino acid changes and substitutions, including conservative changes, which do not significantly alter the activity or binding characteristics of the resulting protein.

Exemplary and preferred conservative amino acid substitutions include any of: glutamine (Q) for glutamic acid (E) and vice versa; leucine (L) for valine (V) and vice versa; serine(S) for threonine (T) and vice versa; isoleucine (I) for valine (V) and vice versa; lysine (K) for glutamine (Q) and vice versa; isoleucine (I) for methionine (M) and vice versa; serine(S) for asparagine (N) and vice versa; leucine (L) for methionine (M) and vice versa; lysine (L) for glutamic acid (E) and vice versa; alanine (A) for serine(S) and vice versa; tyrosine (Y) for phenylalanine (F) and vice versa; glutamic acid (E) for aspartic acid (D) and vice versa; leucine (L) for isoleucine (I) and vice versa; lysine (K) for arginine (R) and vice versa.

Two amino acid sequences are “highly homologous” or “substantially homologous” when at least about 70% of the amino acid residues (preferably at least about 80%, and most preferably at least about 90 or 95%) are identical, or represent conservative substitutions.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

The term “agent” means any molecule, including polypeptides, antibodies, polynucleotides, chemical compounds and small molecules. In particular the term agent includes compounds such as test compounds or drug candidate compounds.

The term “assay” means any process used to measure a specific property of a compound. A “screening assay” means a process used to characterize or select compounds based upon their activity from a collection of compounds.

The term “preventing” or “prevention” refers to a reduction in risk of acquiring or developing a disease or disorder (i.e., causing at least one of the clinical symptoms of the disease not to develop) in a subject that may be exposed to a disease-causing agent, or predisposed to the disease in advance of disease onset.

The term “prophylaxis” is related to and encompassed in the term ‘prevention’, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

“Therapeutically effective amount” means that amount of a drug, compound, antimicrobial, antibody, or pharmaceutical agent that will elicit the biological or medical response of a subject that is being sought by a medical doctor or other clinician. In particular, with regard to bacterial infections and growth of bacteria, the term “effective amount” is intended to include an effective amount of a compound or agent that will bring about a biologically meaningful decrease in the amount of or extent of bacteria present in or infecting an animal. The phrase “therapeutically effective amount” is used herein to mean an amount sufficient to prevent, and preferably reduce by at least about 30 percent, more preferably by at least 50 percent, most preferably by at least 90 percent.

The term “treating” or “treatment” of any disease or infection refers, in one embodiment, to ameliorating the disease or infection (i.e., arresting the disease or growth of the infectious agent or bacteria or reducing the manifestation, extent or severity of at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or infection, either physically, (e.g., stabilization of a discernible symptom), physiologically, (e.g., stabilization of a physical parameter), or both. In a further embodiment, “treating” or “treatment” relates to slowing the progression of a disease or reducing an infection.

The phrase “pharmaceutically acceptable” refers to molecular entities and compositions that are physiologically tolerable and do not typically produce an allergic or similar untoward reaction, such as gastric upset, dizziness and the like, when administered to a human.

Any examples or illustrations given herein are not to be regarded in any way as restrictions on, limits to, or express definitions of any term or terms with which they are utilized. Instead, these examples or illustrations are to be regarded as being described with respect to one particular embodiment and as being illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example,” “for instance,” “e.g.,” and “in one embodiment.” In this specification, groups of various parameters containing multiple members are described. Within a group of parameters, each member may be combined with any one or more of the other members to make additional sub-groups. For example, if the members of a group are a, b, c, d, and e, additional sub-groups specifically contemplated include any one, two, three, or four of the members, e.g., a and c; a, d, and e; b, c, d, and e; etc.

Throughout this specification, quantities are defined by ranges, and by lower and upper boundaries of ranges. Each lower boundary can be combined with each upper boundary to define a range. The lower and upper boundaries should each be taken as a separate element. Two lower boundaries or two upper boundaries may be combined to define a range.

As used herein, “pg” means picogram, “ng” means nanogram, “ug” or “μg” mean microgram, “mg” means milligram, “ul” or “μl” mean microliter, “ml” means milliliter, “l” means liter.

The invention provides nanobodies directed against Campylobacter antigens, paryticularly C. jejuni antigens, selected from one or more of CmeC, CadF, CfrA, CjaD and flagella antigens are provided. In embodiments, combinations of one or more nanobodies directed against one or more of CmeC, CadF, CfrA, CjaD and flagella Campylobacter antigens are provided.

In an embodiment, nanobodies directed against CfrA antigen are provided. Exemplary nanobodies denoted ECM-1G7 and ECM-1C11 are provided. In an embodiment, nanobody denoted ECM-1D6 is also provided.

Selected nanobody sequences against CfrA are set out in FIG. 2. Sequence of the ECM-1C11 nanobody are as follows:

>CmeC-ECM-1C11
(SEQ ID NO: 66)
EVQLVESGGGLVQPGGSLRLSCAASGFTFSDQPMGWYRQAPGQGREWIA
FISSGSGGTTDYKNSVKGRFTISRDNAKNIVYLQMNSLKPDDTARYYCN
FRIDNNYWGQGTQVTVSSAAASGSLEQKLISEEDINGAAHHHHHHGAA
(SEQ ID NO: 78)
EVQLVESGGGLVQPGGSLRLSCAASGFTFSDQPMGWYRQAPGQGREWIA
FISSGSGGTTDYKNSVKGRFTISRDNAKNIVYLQMNSLKPDDTARYYCN
FRIDNNYWGQGTQVTVSSAAASGSLEQ

Sequence of the ECM-1G7 nanobody are as follows:

>CmeC-ECM-1G7
(SEQ ID NO: 67)
EVQLVESGGDLVQDGGSLRLSCAASGGDFRNPVTGWYRQAPGKQRELVG
LITSDGRTNYGDSVMGRFTISMDTAKNTMYLQMNSLKPEDSAVYYCAAS
LLYGGLQFSTNIWGQGTQVTVSSAAASGSLEQKLISEEDLNGAAHHHHH
HGAA
(SEQ ID NO: 78)
EVQLVESGGDLVQDGGSLRLSCAASGGDFRNPVTGWYRQAPGKQRELVG
LITSDGRTNYGDSVMGRFTISMDTAKNTMYLQMNSLKPEDSAVYYCAAS
LLYGGLQFSTNIWGQGTQVTVSSAAASGSLEQ

Sequence of the ECM-1D6 nanobody are as follows:

>CmeC-ECM-1D6
(SEQ ID NO: 1)
EVQLVESGGGLVQAGGSLRLSCAASGGTFSRYMMGWFRQAPGKEREFVA
SIARSGGTYFADSVKGRLTATRDDAKNTMHLQMNSLKPEDTAVYYCAAG
AGGSYPALLDFEYLVWGQGTQVTVSSAAASGSLEQKLISEEDLNGAAHH
HHHHGAA
(SEQ ID NO: 80)
EVQLVESGGGLVQAGGSLRLSCAASGGTFSRYMMGWFRQAPGKEREFVA
SIARSGGTYFADSVKGRLTATRDDAKNTMHLQMNSLKPEDTAVYYCAAG
AGGSYPALLDFEYLVWGQGTQVTVSSAAASGSLEQ

In an embodiment, the nanobodies comprise an amino acid sequence having at least 80%, at least 90%, at least 95%, at least 98%, at least 99% amino acid identity to the sequences of nanobodies selected from ECM-1G7, ECM-1C11 and ECM-1D6.

In an embodiment, nanobodies directed against CadF antigen are provided. Exemplary nanobodies denoted ECF-1F10 and ECF-1D10 are provided. VHH sequences for these antibodies are provided.

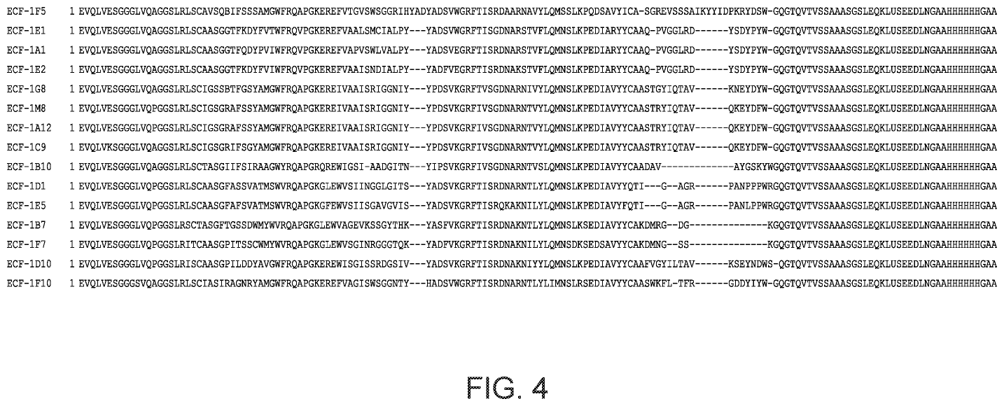

Selected nanobody sequences against CadF are set out in FIG. 4. Sequence of the ECF-1D10 nanobody are as follows:

>CadF-ECF-ID10
(SEQ ID NO: 68)
EVQLVESGGGLVQPGGSLRLSCAASGFTLDDYAVGWFRQAPGK
GEREWLSISSRDGSTVYADSVKGRFTISRDNAKNTYYLQMNSL
KPEDTAVYYCAAAVGYILTRVKSHYNDWSQGTQVTVSSAAASG
SLEQKLISEEDLNGAAHHHHHHGAA
(SEQ ID NO: 81)
EVQLVESGGGLVQPGGSLRLSCAASGFTLDDYAVGWFRQAPGK
EREWLSGISSRDGSTVYADSVKGRFTISRDNAKNTYYLQMNSL
KPEDTAVYYCAAAVGYILTRVKSHYNDWSQGTQVTVSSAAASG
SLEQ

Sequence of the ECF-1F10 nanobody are as follows:

>CadF-ECF-IF10
(SEQ ID NO: 82)
EVQLVESGGGSVQAGGSLRLSCTASIRAGNRYAMGWFRQAPGKER
EFVAGISWSGGNTYHADSVNGRFTISRDNAKNTLYLTMNSLKSED
TAVYYCAASWKPLTFRGDDYTYWGQGTQVTVSSAAASGSLEQKLI
SEEDLNGAAHHHHHHGAA
(SEQ ID NO: 69)
EVQLVESGGGSVQAGGSLRLSCTASIRAGNRYAMGWFRQAPGKER
EFVAGISWSGGNTYHADSVNGRFTISRDNAKNTLYLTMNSLKSED
TAVYYCAASWKPLTFRGDDYTYWGQGTQVTVSSAAASGSLEQ

In an embodiment, the nanobodies comprise an amino acid sequence having at least 80%, at least 90%, at least 95%, at least 98%, at least 99% amino acid identity to the sequences of nanobodies selected from ECF-1F10 and ECF-1D10.

In an embodiment, nanobodies directed against CfrA antigen are provided. Exemplary nanobodies denoted ECA-1G7 are provided. VHH sequences for this antibodies are provided.

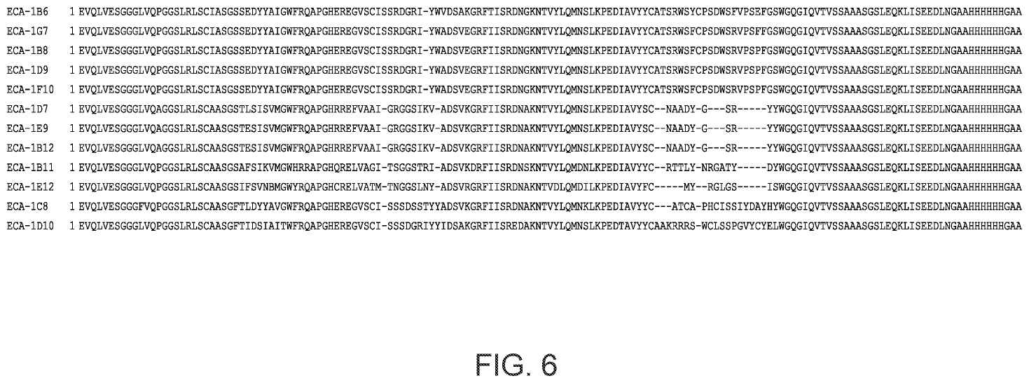

Selected nanobody sequences against CfrA are set out in FIG. 6. Sequence of the ECA-1G7 nanobody are as follows:

>CfrA-ECA-1G7
(SEQ ID NO: 29)
EVQLVESGGGLVQPGGSLRLSCTASGSSLDYYAIGWFRQAPGKER
EGVSCISSRDGRIYWADSVEGRFTISRDNGKNTVYLQMNSLKPED
TAVYYCATSRWSFCPSDWSPVPSPFGSWGQGTQVTVSSAAASGSL
EQKLISEEDLNGAAHHHHHHGAA
(SEQ ID NO: 83)
EVQLVESGGGLVQPGGSLRLSCTASGSSLDYYAIGWFRQAPGKER
EGVSCISSRDGRIYWADSVEGRFTISRDNGKNTVYLQMNSLKPED
TAVYYCATSRWSFCPSDWSPVPSPFGSWGQGTQVTVSSAAASGSL
EQ

In an embodiment, the nanobodies comprise an amino acid sequence having at least 80%, at least 90%, at least 95%, at least 98%, at least 99% amino acid identity to the sequences of nanobody ECA-1G7.

In an embodiment, nanobodies directed against CjaD antigen are provided. Exemplary nanobodies denoted ECD-1D3 and ECD-1A7 are provided. VHH sequences for these antibodies are provided.

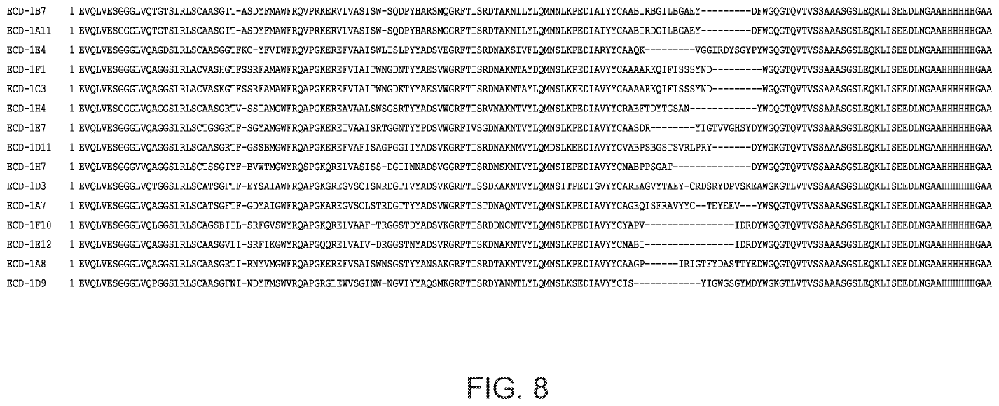

Selected nanobody sequences against CjaD are set out in FIG. 8.

Sequence of the ECD-1D3 nanobody are as follows:

>CjaD-ECD-1D3
(SEQ ID NO: 49)
EVQLVESGGGLVQTGGSLRLSCATSGFTFEYSAIAWFRQAPGKGR
EGVSCISNRDGTTVYADSVKGRFTISSDNAKNTVYLQMNSLTPED
TGVYYCAADAGVYTADYCRDSRYDPVSKDAWGKGTLVTVSSAAAS
GSLEQKLISEEDLNGAAHHHHHHGAA
(SEQ ID NO: 84)
EVQLVESGGGLVQTGGSLRLSCATSGFTFEYSAIAWFRQAPGKGR
EGVSCISNRDGTTVYADSVKGRFTISSDNAKNTVYLQMNSLTPED
TGVYYCAADAGVYTADYCRDSRYDPVSKDAWGKGTLVTVSSAAAS
GSLEQ

Sequence of the ECD-1A7 nanobody are as follows:

>CjaD-ECD-1A7
(SEQ ID NO: 53)
EVQLVESGGGLVQAGGSLRLSCATSGFTPGDYAIGWFRQAPGKAR
EGVSCLSTRDGTTYYADSVKGRFTISTDNAQNTVYLQMNSLKPED
TAVYYCAGEQISFRAVYYCTEYEPVYWSQGTQVTVSSAAASGSLE
QKLISEEDLNGAAHHHHHHGAA
(SEQ ID NO: 85)
EVQLVESGGGLVQAGGSLRLSCATSGFTPGDYAIGWFRQAPGKAR
EGVSCLSTRDGTTYYADSVKGRFTISTDNAQNTVYLQMNSLKPED
TAVYYCAGEQISFRAVYYCTEYEPVYWSQGTQVTVSSAAASGSLE
Q

In an embodiment, the nanobodies comprise an amino acid sequence having at least 80%, at least 90%, at least 95%, at least 98%, at least 99% amino acid identity to the sequences of nanobodies selected from ECD-1D3 and ECD-1A7.

In an embodiment, nanobodies directed against C. jejuni flagella as antigen are provided. Exemplary nanobodies denoted ECF-1C4, ECF-1F4, ECF-1B6, ECF1G8 and ECF-1D9 are provided. VHH sequences for these antibodies are provided.

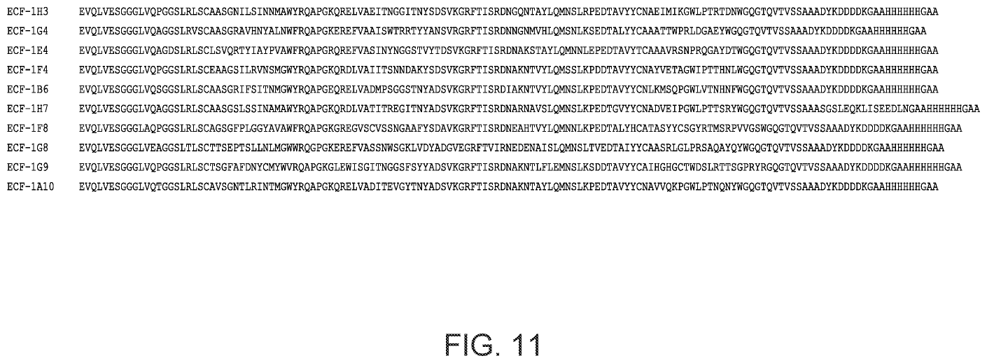

Selected nanobody sequences against C. jejuni flagella are set out in FIG. 11. Sequence of the ECF-1C4 nanobody are as follows:

>Flagella-ECF-1C4
(SEQ ID NO: 73)
EVQLVESGGGLVQAGGSLRVSCAASGRAVHNYALNWFRQAPGKER
EFVAAISWTRRTYYANSVRGRFTISRDNNGNMVHLQMSNLKSEDT
ALYYCAAATTWPRLDGAEYWGQGTQVTVSSAAADYKDDDDKGAAH
HHHHHGAA
(SEQ ID NO: 86)
EVQLVESGGGLVQAGGSLRVSCAASGRAVHNYALNWFRQAPGKER
EFVAAISWTRRTYYANSVRGRFTISRDNNGNMVHLQMSNLKSEDT
ALYYCAAATTWPRLDGAEYWGQGTQVTVSS

Sequence of the ECF-1F4 nanobody are as follows:

>Flagella-ECF-1F4
(SEQ ID NO: 74)
EVQLVESGGGLVQPGGSLRLSCEAAGSILRVNSMGWYRQAPGKQR
DLVAIITSNNDAKYSDSVKGRFTISRDNAKNTVYLQMSSLKPDDT
AVYYCNAYVETAGWIPTTHNLWGQGTQVTVSSAAADYKDDDDKGA
AHHHHHHGAA
(SEQ ID NO: 87)
EVQLVESGGGLVQPGGSLRLSCEAAGSILRVNSMGWYRQAPGKQR
DLVAIITSNNDAKYSDSVKGRFTISRDNAKNTVYLQMSSLKPDDT
AVYYCNAYVETAGWIPTTHNLWGQGTQVTVSSAAA

Sequence of the ECF-1B6 nanobody are as follows:

>Flagella-ECF-1B6
(SEQ ID NO: 75)
EVQLVESGGGLVQSGGSLRLSCAASGRIFSITNMGWYRQAPGEQR
ELVADMPSGGSTNYADSVKGRFTISRDIAKNTVYLQMNSLKPEDT
AVYYCNLKMSQPGWLVTNHNFWGQGTQVTVSSAAADYKDDDDKGA
AHHHHHHGAA
(SEQ ID NO: 88)
EVQLVESGGGLVQSGGSLRLSCAASGRIFSITNMGWYRQAPGEQR
ELVADMPSGGSTNYADSVKGRFTISRDIAKNTVYLQMNSLKPEDT
AVYYCNLKMSQPGWLVTNHNFWGQGTQVTVSSAAA

Sequence of the ECF-1G8 nanobody are as follows:

>Flagella-ECF-1G8
(SEQ ID NO: 76)
EVQLVESGGGLVEAGGSLTLSCTTSEPTSLLNLMGWWRQGPGKER
EFVASSNWSGKLVDYADGVEGRFTVIRNEDENAISLQMNSLTVED
TAIYYCAASRLGLPRSAQAYQYWGQGTQVTVSSAAADYKDDDDKG
AAHHHHHHGAA
(SEQ ID NO: 89)
EVQLVESGGGLVEAGGSLTLSCTTSEPTSLLNLMGWWRQGPGKER
EFVASSNWSGKLVDYADGVEGRFTVIRNEDENAISLQMNSLTVED
TAIYYCAASRLGLPRSAQAYQYWGQGTQVTVSSAAA

Sequence of the ECF-1D9 nanobody are as follows:

>Flagella-ECF-1D9
(SEQ ID NO: 77)
EVQLVESGGGLVQPGGSLRLSCATSGFAFDNYCMYWVRQAPGKGL
EWISGITNGGSFSYYADSVKGRFTISRDNAKNTLFLEMNSLKSDD
TAVYYCAIHGHGCTWDSLRTTSGPRYRGQGTQVTVSSAAADYKDD
DDKGAAHHHHHHGAA
(SEQ ID NO: 90)
EVQLVESGGGLVQPGGSLRLSCATSGFAFDNYCMYWVRQAPGKGL
EWISGITNGGSFSYYADSVKGRFTISRDNAKNTLFLEMNSLKSDD
TAVYYCAIHGHGCTWDSLRTTSGPRYRGQGTQVTVSSAAA

In an embodiment, the nanobodies comprise an amino acid sequence having at least 80%, at least 90%, at least 95%, at least 98%, at least 99% amino acid identity to the sequences of nanobodies selected from ECF-1C4, ECF-1F4, ECF-1B6, ECF1G8 and ECF-1D9.

In embodiments, combinations of one or more nanobodies directed against one or more of CmeC, CadF, CfrA, CjaD and flagella Campylobacter antigens are provided, particularly combinations of the antibodies selected and provided above. In an embodiment, the invention provides and relates to combinations of two or more of CmeC, CadF, CfrA, CjaD and flagella Campylobacter antigens are provided, particularly combinations of the antibodies selected and provided above. In an embodiment, the invention provides and relates to combinations of nanobodies directed to each of CmeC, CadF, CfrA, CjaD and flagella Campylobacter antigens, particularly combinations of the antibodies selected and provided above. Thus, a combination of a nanobody directed against CmeC antigen, CadF antigen, CfrA antigen, CjaD antigen, and flagellar antigen is provided. The combination may be achieved by administration of one or more or of multiple nanobodies in a single composition. The combination may be achieved by expression of one or more or of multiple nanobodies in an animal.

The herein flagellar nanobodies are generated using, for example, using native flagella. Native flagella can be glycosylated. Multimeric nanobodies are generated to further enhance the potency/in vivo efficacy. Multiple copies of nanobodies are combined using a GS or similar linker where the number of copies can vary from 2 to 5.

By “substantially as set out” it is meant that variable region sequences, and/or particularly the CDR sequences, of the invention will be either identical or highly homologous to the specified regions of FIG. 2, 4, 6, 8, 10 or 11. By “highly homologous” it is contemplated that only a few substitutions, preferably from 1 to 8, preferably from 1 to 5, preferably from 1 to 4, or from 1 to 3, or 1 or 2 substitutions may be made in the variable region sequence and/or in the CDR sequences. The term substantially set out as includes particularly conservative amino acid substitutions which do not materially or significantly affect the specificity and/or activity of the instant nanobodies. Conservative and non-conservative amino acid substitutions are contemplated herein for the variable region sequences and also for the CDR region sequences.

Substitutions may be made in the variable region sequence outside of the CDRs so as to retain the CDR sequences. Thus, changes in the variable region sequence or alternative non-homologous or veneered variable region sequences may be introduced or utilized, such that the CDR sequences are maintained and the remainder of the variable region sesuence may be substituted.

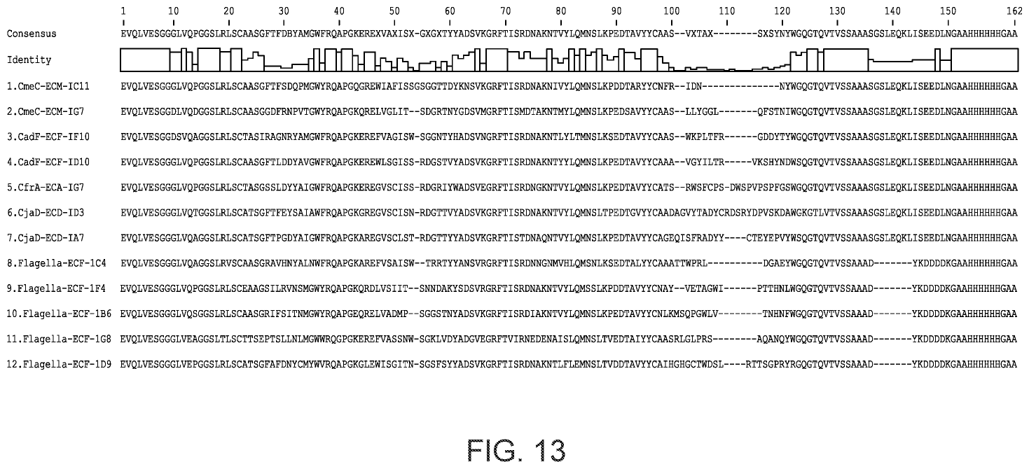

There are several recognized and known methods and approaches to determine the CDRs in an antibody. The most commonly used CDR identification methods at present are Kabat (Wu T T, Kabat E A (1970) J Exp Med 132:211-250; Kabat E A et al (1983) Sequence of Proteins of Immunological Interest. Bethesda: National Institute of Health), IMGT (Lefranc M P et al (2003) Dev Comp Immunol 27:55-77) and Chothia (Chothia C, Lesk A M (1987) J Mol Biol 196:901-917; Chothia C et al (1989) Nature 342:877-883; Lefranc M P et al (2003) Dev Comp Immunol 27:55-77). Each of these methods has devised a unique residue numbering scheme according to which it numbers the hypervariable region residues and the beginning and ending of each of the CDRs is then determined according to certain key positions. IMGT and Kabat systems were utilized in the present studies. While these different approaches may identify slightly offset CDR sequences, they generally provide overlapping sequences and amino acids and can be useful in combination to identify amino acids which should be maintained or conserved and those that may be suitable for variation or alteration while maintaining binding. FIG. 13 provided herein shows a comparison of selected nanobodies agsinst distinct Campylobacter protein targets. This comparison points to regions of distinct sequence, which provides one skilled in the art direction as to the applicable heavy chain VHH CDRs and CDR1, CDR2, and CDR3 regions and sequences.

Additional residues at the N-terminal or C-terminal end of the substantial part of the variable domain may be those not normally associated with naturally occurring variable domain regions. For example, construction of specific binding members of the present invention made by recombinant DNA techniques may result in the introduction of N- or C-terminal residues encoded by linkers introduced to facilitate cloning or other manipulation steps. Other manipulation steps include the introduction of linkers to join variable domains of the invention to further protein sequences including immunoglobulin heavy chains, other variable domains (for example in the production of diabodies) or protein labels as provided herein and/or known to those of skill in the art.

The antibodies, or any fragments thereof, may be conjugated or recombinantly fused to any cellular toxin, bacterial or other, e.g. pseudomonas exotoxin, ricin, or diphtheria toxin. The part of the toxin used can be the whole toxin, or any particular domain of the toxin. Bi- and tri-specific multimers can be formed by association of different scFv molecules and have been designed as cross-linking reagents for T-cell recruitment into tumors (immunotherapy), viral retargeting (gene therapy) and as red blood cell agglutination reagents (immunodiagnostics), see e.g. Todorovska et al., J Immunol Methods. 2001 Feb. 1; 248 (1-2): 47-66; Tomlinson et al., Methods Enzymol. 2000; 326:461-79; McCall et al., J Immunol. 2001 May 15; 166 (10): 6112-7.

Nanobodies of the invention may be labelled with a detectable or functional label. Detectable labels include, but are not limited to, radiolabels such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²¹I, ¹²⁴I, ¹²⁵I, ¹³¹I, ¹¹¹In, ¹¹⁷Lu, ²¹¹At, ¹⁹⁸Au, ⁶⁷Cu, ²²⁵Ac, ²¹³Bi, ⁹⁹Tc and ¹⁸⁶Re, which may be attached to antibodies of the invention using conventional chemistry known in the art of antibody imaging. Labels also include fluorescent labels (for example fluorescein, rhodamine, Texas Red) and labels used conventionally in the art for MRI-CT imaging. They also include enzyme labels such as horseradish peroxidase, β-glucoronidase, β-galactosidase, urease. Labels further include chemical moieties such as biotin which may be detected via binding to a specific cognate detectable moiety, e.g. labelled avidin. Functional labels include substances which are designed to be targeted to the site of a tumor to cause destruction of tumor tissue. Such functional labels include cytotoxic drugs such as 5-fluorouracil or ricin and enzymes such as bacterial carboxypeptidase or nitroreductase, which are capable of converting prodrugs into active drugs at the site of a tumor.

As used herein, an “epitope” is a term in the art and refers to a localized region of an antigen to which an antibody can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In certain embodiments, the epitope to which an antibody binds can be determined by, e.g., NMR spectroscopy, X-ray diffraction crystallography studies, ELISA assays, hydrogen/deuterium exchange coupled with mass spectrometry (e.g., MALDI mass spectrometry), array-based oligo-peptide scanning assays, and/or mutagenesis mapping (e.g., site-directed mutagenesis mapping).

In certain aspects, competition binding assays can be used to determine whether an antibody or nanobody is competitively blocked, e.g., in a dose dependent manner, by another antibody or nanobody for example, an antibody binds essentially the same epitope, or overlapping epitopes, as a reference antibody, when the two antibodies recognize identical or sterically overlapping epitopes in competition binding assays such as competition ELISA assays, which can be configured in all number of different formats, using either labeled antigen or labeled antibody. In a particular embodiment, an antibody or nanobody can be tested in competition binding assays with an antibody described herein.

Competition binding assays also can be used to determine whether two antibodies have similar binding specificity for an antigen or an epitope, including a particular epitope on an antigen or protein target. Competitive binding can be determined in an assay in which the immunoglobulin under test inhibits specific binding of another antibody to a common antigen or target antigen.

Assays known to one of skill in the art or described herein (e.g., X-ray crystallography, ELISA assays, etc.) can be used to determine if two antibodies bind to the same epitope. Biacore assays can be used to assess and determine competitive binding and also epitope binding. Biacore can be utilized to determine the extent to which different antibodies interact with a single antigen or epitope, to assess protein or antibody-protein interactions, and to determine binding affinity.

Immunoconjugates or antibody fusion proteins of the present invention, wherein the nanobodies of the present invention are conjugated or attached to other molecules or agents further include, but are not limited to binding members conjugated to a immunomodulator, antibacterial agent, antibiotic, or drug.

Nanobodies of the present invention may be administered to an animal in need of treatment via any suitable route, including orally, by spray administration, by injection, including intreperitoneally, intramuscularly, subcutaneous, intravenous, into the bloodstream or intestine or gut, or directly into the gut. The precise dose will depend upon a number of factors, including whether the nanobody is for diagnosis or for treatment, the dose methodology or administration type, and the applicable animal.

In an embodiment, administered comprises in ovo administration. In an embodiment, administered comprises spray administration. In an embodiment, administered comprises immersion, intranasal, intramammary, topical, or inhalation.

The compositions may further include one or more component or additive. The one or more component or additive may be a component or additive to facilitate administration, for example by way of a stabilizer or vehicle, or by way of an additive to enable administration to an animal such as by any suitable administrative means, including in aerosol or spray form, in water, in feed or in an injectable form. Administration to an animal may be by any known or standard technique. These include oral ingestion, gastric intubation, or broncho-nasal spraying. The compositions disclosed herein may be administered by immersion, intranasal, intramammary, topical, mucosally, or inhalation. When the animal is a bird the treatment may be administered in ovo or by spray inhalation.

Nanobodies of the present invention will usually be administered in the form of a pharmaceutical composition, which may comprise at least one component in addition to the specific nanobody. Thus pharmaceutical compositions or immunological compositions according to the present invention, and for use in accordance with the present invention, may comprise, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. intravenous, or by deposition at a tumor site.

A composition of the present invention may be administered alone or in combination with other treatments, therapeutics or agents, either simultaneously or sequentially dependent upon the condition to be treated.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, injection, or injection at the site of affliction, the active ingredient may be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

In addition, the present invention contemplates and includes therapeutic compositions for the use of the nanobody(ies) in combination with conventional antibacterial therapy. The present invention further contemplates therapeutic compositions useful in practicing the therapeutic methods of this invention. A subject therapeutic composition includes, in admixture, a pharmaceutically acceptable excipient (carrier) and one or more of a nanobody as described herein as an active ingredient.

The preparation of therapeutic compositions which contain polypeptides, analogs or active fragments as active ingredients is well understood in the art. Typically, such compositions are prepared as injectables, either as liquid solutions or suspensions. However, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

A polypeptide, analog or active fragment can be formulated into the therapeutic composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The compositions are administered in a manner compatible with the dosage formulation, and in a therapeutically effective amount. The method and mode of administration may be adjusted or different methods and modes may be applied depending on the animal to be administered. For instance, administration in feed or spray or in water provided or applied to animals or eggs is contemplated. Administration using any of various vehicles is contemplated. Administration may include expression by virtue of an encoding plasmid, vector, nucleic acid etc. The quantity to be administered depends on the subject or animal to be treated, capacity of the subject's or animal's immune system to utilize the active ingredient, etc. Precise amounts of active ingredient required to be administered depend on the judgment of the practitioner and are peculiar to each individual. Suitable regimes for initial administration and follow on administration are also variable, and may include an initial administration followed by one or more repeated dose or doses.

Diagnostic applications of the nanobodies of the present invention, particularly antibodies and fragments thereof, include in vitro and in vivo applications well known and standard to the skilled artisan and based on the present description. Diagnostic assays and kits for in vitro assessment and evaluation of bacteria or bacterial infection or colonization may be utilized to diagnose, evaluate and monitor animal or patient samples including those known to have or suspected of being infected with Campylobacter or having bacterial gastroenteritis.

The present invention further provides an isolated nucleic acid encoding a nanobody of the present invention. Nucleic acid includes DNA and RNA. In a preferred aspect, the present invention provides a nucleic acid which codes for a polypeptide of the invention as defined above, including a polypeptide as provided and described herein or as set out in FIG. 2, 4, 6, 8 10 or 11.

The present invention also provides constructs in the form of plasmids, vectors, transcription or expression cassettes which comprise at least one polynucleotide as above. The present invention also provides a recombinant host cell which comprises one or more constructs as above. A nucleic acid encoding any specific binding member as provided itself forms an aspect of the present invention, as does a method of production of the specific binding member which method comprises expression from encoding nucleic acid therefor. Expression may conveniently be achieved by culturing under appropriate conditions recombinant host cells containing the nucleic acid. Following production by expression a specific binding member may be isolated and/or purified using any suitable technique, then used as appropriate.

Systems for cloning and expression of a polypeptide in a variety of different host cells are well known. Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. ‘phage, or phagemid, as appropriate.

Thus, a further aspect of the present invention provides a host cell containing nucleic acid as disclosed herein. A still further aspect provides a method comprising introducing such nucleic acid into a host cell. The introduction may employ any available technique. The introduction may be followed by causing or allowing expression from the nucleic acid, e.g. by culturing host cells under conditions for expression of the gene. The present invention also provides a method which comprises using a construct as stated above in an expression system in order to express a specific binding member or polypeptide as above. Any of a wide variety of expression control sequences—sequences that control the expression of a DNA sequence operatively linked to it—may be used in these vectors to express the DNA sequences of this invention. A wide variety of unicellular host cells are also useful in expressing the DNA sequences of this invention. These hosts may include well known eukaryotic and prokaryotic hosts, such as strains of E. coli, Pseudomonas, Bacillus, Streptomyces, fungi such as yeasts, and animal cells, such as CHO, YB/20, NSO, SP2/0, R1.1, B-W and L-M cells, African Green Monkey kidney cells (e.g., COS 1, COS 7, BSC1, BSC40, and BMT10), insect cells (e.g., Sf9), and human cells and plant cells in tissue culture.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### Example 1

### Anti-Campylobacter Nanobodies

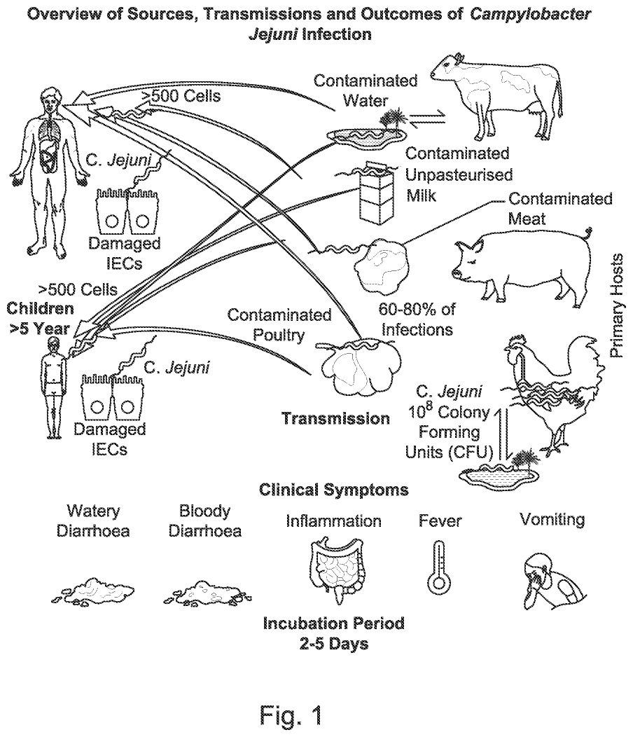

Campylobacter bacteria remain a major public health concern. Campylobacter are the leading cause of bacterial gastroenteritis in the world. There are 31 different species, with C. jejuni being the most clinically relevant species responsible for up to 80-90% of Campylobacter infections. Campylobacter are normal or ordinary inhabitants of a variety of food producing animals including poultry, swine (pigs) and cattle, and chickens are the primary source of human infections. An overview of sources, transmissions an outcomes of Campylobacter jejuni infection is provided in FIG. 1.

Standard vaccination approaches to C. jejuni infection or colonization, particularly in animals for food, including poultry, are not very effective and have not been implemented. The immune response in chickens is not fast or robust. On hatching, chicken hatchlings are not initially colonized by the bacteria but then quickly high colonization is observed after the first two weeks and into 6 weeks of age when they are harvested as a food source. Prevalence rates of commercial broiler flocks infected with Campylobacter jejuni in the U.S. and throughout the world in Europe, Canada and Israel range from 30-90%.

Nanobodies, are single domain (Heavy chain variable region VHH) antibodies (Ward, E. S. et al., Nature 341, 544-546 (1989)). Single domain antibodies were initially isolated from camelid animals and have been designated interchangeably as camelid antibodies, nanobodies or VHH. A VHH antibody corresponds to the variable region of an antibody heavy chain and has a very small size of around 15 kDa-hence the name “nanobody”. The advantage of these antibody-derived molecules is their small size which can enable their binding to hidden epitopes not accessible to whole antibodies. In the context of therapeutic applications, a small molecular weight also means an efficient penetration and fast clearance. Both scFv and VHH nanobodies can be linked to the Fc fragment of the desired species and keep their specificity and binding properties and are then termed minibody.

Nanobodies are small, low molecular weight, single-domain, heavy-chain only antibody. Owing to its smaller size, genes of these proteins can be readily cloned and manipulated to present on plasmids or in integrated form, expression vector, etc. Therefore, by using molecular cloning techniques, nanobodies against various antigens can be presented, even on a single or multiple constructs, and be provided to a target region or to the systemic circulation.

Campylobacter targets with proven and key roles in Campylobacter colonization were selected for the generation of specific and directed nanobodies. The antigen targets are CfrA, CmeC, CjaD, CadF and flagella. CfrA is involved in high affinity iron acquisition, required for chicken colonization and elicits a strong immune response and protection after vaccination. TonB3-dependent iron acquisition; required for chicken colonization; oral delivery of Lactococcus lactis expressing ferric enterobactin receptor CfrA of Campylobacter jejuni reduces C. jejuni colonization (Palyada et al., 2004, J Bacteriol; Wang et al., 2016, Wei Sheng Wu Xue Bao; Naikare et al., 2013, Metallomics; Zeng et al., 2009. Infect Immun.).

CmeC is a component of the multidrug efflux pump, required for chicken colonization, and elicits strong immune response and protection after vaccination. It is a multidrug resistance gene. Subcutaneous vaccination of chickens with rCmeC stimulated both serum IgG and IgA responses (Zeng et al., 2010. J Vaccines Vaccin; Lin et al., 2002, Antimicrob Agents Chemother.)

CjaD is a peptidoglycan binding protein and strong immune response and protection after vaccination. In ovo vaccination with CjaD provides significant protection against heterologous challenge. L. lactis harboring the surface-exposed rCjaAD antigen affords protection. Salmonella vectored vaccine confers protection against C. jejuni colonization (Kobierecka et al., 2016a, Front. Microbiol; Kobierecka et al., 2016b, Fron. Microbiol; Layton et al., 2011, Clin Vaccine Immunol).

CadF is an outer membrane adhesin for fibronectin binding. It is required for chicken colonization and elicits strong immune response and protection after vaccination. It is required for Campylobacter adhesion to fibronectin. Vaccination results in C. jejuni specific IgY antibodies and protection against C. jejuni colonization; induce massive immune response (Neal Mckinney et al., 2014; Meunier et al., 2016, J Immunol Res; Shams et al., 2016, J Microbiol.; Krause-Gruszczynska et al., 2007, FEMS Microbiol Letters)

Flagella of C. jejuni function importantly in motility and secretion and chicken colonization. Flagella are required for motility in the animal host gut mucus layer and are also involved in the Campylobacter secretion system. Recombinant flagellar proteins react with chicken serum challenged with C. jejuni. Recombinant FlaA protected mice against C. jejuni colonization (Yeh et al., 2015, Arch. Microbiol; Lee et al., 1999, Infect. Immun; Meunier et al., 2017, PLOS ONE).

Llamas were immunized with CfrA, CmeC, CjaD, CadF and flagella, VHHs were purified and selected, evaluated for immune response against the selected antigens, VHH sequences determined and compared, and dose response of selected VHH clones determined. Llama immunization and selection and assessment of nanobody clones are described in the next examples, Examples 2-6.

### Example 2

### Selection and Screening of VHHs Against CmeC

Immunization-CmeC protein was provided as antigen and immunization was carried out in llamas SNL148 and SNL149. Both llamas were immunized in 4 injections at day 0, 14, 28 and 35 with a large bleed at day 43 from which RNA was isolated.

Immune response—The immune response of the llamas was tested in an ELISA on CmeC with the sera of the llamas of day 0, day 28 and day 43. MaxiSorp plates were coated with 200 ng antigen per well overnight at 4° C. After three times washing with PBS containing 0.05% Tween-20 the plates were blocked with 4% milk powder in PBS (MPBS), Next a serial dilution of the sera in 1% MPBS was added to the wells and incubated for 1 hour. Unbound VHH were removed during washing with PBS-Tween. Subsequently, bound VHH were detected with an anti-VHH antibody and anti-rabbit antibody coupled to a peroxidase. Binding of the VHH was quantified by the colorimetric reaction of O-phenylenediamine (OPD) in the presence of H₂O₂ at 490 nm (data not shown). Both llamas showed a good immune response. A small response was seen for the day 0 sera of both llamas, but this looks irrelevant compared to the response that is seen with the sera of day 28 and day 43. The response shown with the sera of day 28 is similar to the response from the sera of day 43. This is the case for both llamas.

### Library Construction of SNL148 Day 43 and SNL149 Day 43

RNA Isolation and cDNA Synthesis

Immunizations and RNA preparation were done at Eurogentec. 5 μl of the obtained RNA was loaded on gel after precipitation, before use. Intact 28S and 18S rRNA were clearly visible. The remaining of the RNA was stored in 70% EtOH, containing 200 mM NaAc at −80° C.

After precipitation, the RNA concentrations were measured again and about 40 μg RNA (4 reactions of 10 μg each) was transcribed into cDNA using a reverse transcriptase Kit (Invitrogen). The cDNA was cleaned on Macherey Nagel PCR clean-up columns. IG H (both conventional and heavy chain) fragments were amplified using primers annealing at the leader sequence region and at the CH2 region. 5 μl was loaded onto a 1% TBE agarose gel for a control of the amplification which showed that the two DNA fragments (˜700 bp and ˜900 bp) were amplified representing the VHH and VH, respectively (data not shown).

After the control, the remaining of the samples were loaded on a 1% TAE agarose gel. The 700 bp fragment was excised from the gel and purified. About 80 ng was used as a template for the nested PCR (end volume 800 μl). The amplified fragment was cleaned on Macherey Nagel PCR cleaning columns and eluted in 120 μl. The eluted DNA was digested with first SfiI and next BstEII. As a control of the restriction digestion, 5 μl of this mixture was loaded onto a 1.5% TBE agarose gel which showed that the DNA has been properly digested and the 400 bp DNA band was clearly visible (data not shown).

After the restriction digestion, the samples were loaded on a 1.5% TAE agarose gel. The 400 bp fragment was excised from the gel and purified on Machery Nagel gel extraction columns. The purified 400 bp fragments (˜330 ng) were ligated into the phagemid pUR8100 vector (˜1 μg) and transformed into TG1. The transformed TG1 were titrated using 10-fold dilutions. 5 μl of the dilutions were spotted on LB-agar plates supplemented with 100 μg/ml ampicillin and 2% glucose (data not shown).

### Library Size

The number of transformants was calculated from the spotted dilutions of the rescued TG1 culture (total end volume is 8 ml). The titer of the library was calculated by counting colonies in the highest dilution and using the formula below:

$$Library size = ( amount of colonies ) * ( dilution ) * 8 ( ml ) / 0.005 ( ml ; spotted volume )$$

Table 1 shows the calculated library size, including the measured OD600 of the culture. All libraries were of good size with more than 107 clones per library. The bacteria were stored in 2×YT medium supplemented with 20% glycerol, 2% glucose and 100 μg/ml ampicillin at −80° C.

TABLE 1
The size and the measured OD600 of the libraries.

| Llama | Library size | OD600 |
|---|---|---|
| SNL148 day 43 | 1.12 × 109 | 82.1 |
| SNL149 day 43 | 1.28 × 109 | 89.5 |

Insert frequency—The insert frequency was determined by picking 24 different clones from transformations out of each library and performing a colony PCR. Bands of ˜700 bp indicate a cloned VHH fragment. Bands of ˜300 bp indicate an empty plasmid. All PCR reactions delivered a DNA fragment of ˜700 bp. The insert frequency of both library SNL148 day 43 and library SNL149 day 43 is 100%.

### Phage Production and Selection

Phages were produced from the libraries as outlined below: E. coli TG1 containing libraries SNL148 day 43 and SNL149 day 43 were diluted from the glycerol stock up to an OD600 of 0.05 in 2×YT medium containing 2% glucose and 100 μg/ml ampicillin, while the number of bacteria of the inoculum was 10× the library size (>108 bacteria inoculum), and grown at 37° C. for 2 hours to reach an OD600 of ˜0.5. Subsequently, about 7 ml of the cultures were infected with helper phage VCS M13 using a MOI (multiplicity of infection) of 100 for 30 minutes standing at 37° C. TG1 bacteria were spun down and resuspended into 50 ml fresh 2×YT medium supplemented with both ampicillin (100 μg/ml) and kanamycin (25 μg/ml) and grown overnight at 37° C., shaking. Produced phages were precipitated from the supernatant of the cultures using PEG-NaCl precipitation. Titers of the produced phages were calculated by serial dilution of the phage sample and infection of E. coli TG1, which were sufficient for selection. Titers of the libraries were 1×1012/ml for SNL 148 day 43 and 8×1011/ml for SNL 149 day 43, respectively (data not shown).

20 μl of the precipitated phages (˜1011 phages, which is >1000-fold the diversity of the libraries) were applied to wells coated with CmeC. In short, for each library, 100 μl antigen was coated on the MaxiSorp plate overnight at 2 concentrations 5 μg/ml and 0.5 μg/ml. As a negative control, one well was incubated with PBS only. Next day after removal of non-bound antigen, the plate was washed three times with PBS and blocked with 4% milk powder in PBS (MPBS). At the same time freshly precipitated phages were pre-blocked with 2% MPBS for 30 minutes. Pre-blocked phages were incubated with directly coated CmeC for 2 hours. Upon extensive washing with PBS-Tween and PBS, bound phages were eluted with 0.1M TEA-solution and subsequently neutralized with 1M Tris/HCl pH7.5. Eluted phages were serially diluted and then used to infect TG1 bacteria and spotting on LB-agar plates supplemented with 2% glucose and 100 μg/ml ampicillin and incubated overnight at 37° C. (data not shown).

For both libraries some aspecific binding phages from the non-coated wells are eluted, but more binding phages were eluted from the coated wells. Also the output of both phages show a concentration dependent enrichment between the coated wells. Glycerol stocks were prepared from all outputs rescued by infection of E. coli TG1 and stored at −80° C. Simultaneously, TG1 cultures infected with the output of the selection on 5 μg/ml CmeC (highest coating) were used for phage production of SNL 148 day 43 and SNL149 day 43 sub libraries in order to perform the 2^{nd} round of selection. Overnight grown rescued outputs were diluted 100-fold in 5 ml fresh 2×YT medium supplemented with 2% glucose and 100 μg/ml ampicillin and grown for 2 hours until log-phase. Subsequently 1 μl of helper phage VCS M13 was added and incubated at 37° C. for 30 minutes. Cultures were allowed to produce phages overnight at 37° C. Produced phages were precipitated from the supernatant of the cultures using PEG-NaCl precipitation.

1 μl of the precipitated phages was applied to wells coated with CmeC as indicated below: CmeC was coated on the MaxiSorp plate overnight at 3 concentrations (5 μg/ml, 0.5 μg/ml and 0.05 μg/ml). As a negative control, one well was incubated with PBS only. Next day, after removal of non-bound antigen, the plate was washed three times with PBS and blocked with 4% MPBS. At the same time freshly precipitated phages were pre-blocked in 2% MPBS for 30 minutes as described above. Pre-blocked phages were incubated with directly coated CmeC for 2 hours. Upon extensive washing with PBS-Tween and PBS, bound phages were eluted with 0.1M TEA-solution and subsequently neutralized with 1M Tris/HCl pH7.5. Eluted phages were serially diluted and then used to infect TG1 cells and spotting on LB-agar plates supplemented with 2% glucose and 100 g/ml ampicillin and incubated overnight at 37° C. (data not shown).

For both libraries almost no aspecific binding phages were eluted from the non-coated wells. A high output of binding phages eluted from the coated wells, which show a concentration dependent enrichment, is shown for both libraries, indicating that specific binding phages were eluted.

Screening after 2 Rounds of Phage Display Selections

After the 2^{nd} round of phage display selection, glycerol stocks were prepared from all outputs rescued by infection of E. coli TG1 and stored at −80° C. in the same way as for the outputs obtained after the 1^{st} round of phage display selection. Subsequently all rescued outputs of the 2^{nd} round of selection on CmeC were plated out in order pick single colonies, 92 single clones were picked for the master plate. These were grown in a 96-wells plate (master plate ECM-1). This master plate will be used to produce periplasmic fractions containing monoclonal VHHs for screening of binders.

The master plate was cultivated at 37° C. in 2×YT medium supplemented with 2% glucose and 100 μg/ml ampicillin and stored at −80° C. after addition of glycerol to a final concentration of 20%. For the production of periplasmic fractions, master plate ECM-1 was duplicated into a deep well plate containing 1 ml 2×YT medium supplemented with 0.1% glucose and 100 g/ml ampicillin and grown for 3 hours at 37° C. before adding 1 mM IPTG for induction of VHH expression. The VHH expression was conducted overnight at room temperature.

Periplasmic fractions were prepared by collecting the bacteria by centrifugation and their resuspension into 120 μl PBS. After freezing, bacteria were thawed and centrifuged to separate the soluble periplasmic fraction containing the VHH from the cell debris (pellet).

To test the binding specificity of the monoclonal VHHs, 200 ng/well of CmeC was coated overnight in PBS onto MaxiSorp plates at 4° C. The coated plates were washed and subsequently

blocked using 4% MPBS. 25 μl of the periplasmic fractions was added to each well and incubated for 1 hour in 1% MPBS. Unbound VHHs were removed during washing with PBS containing 0.05% Tween-20. Subsequently, bound VHHs were detected with an anti-VHH antibody and anti-rabbit antibody coupled to a peroxidase. Binding of the VHHs was quantified by the colorimetric reaction of OPD in the presence of H₂O₂ at 490 nm (data not shown).

Most of the selected clones were able to bind specifically to CmeC. There does not seem to be a difference in the clones picked from the two different libraries. The difference in output of library SNL149 day 43 from 5 μg/ml CmeC (column 7 and 8) and 0.05 μg/ml (column 11 and 12) shows that selection for affinity by lowering the antigen concentration clearly works well.

### Sequence Analysis of VHHs

Based on the ELISA results, clones ECM-1C1, ECM-1F2, ECM-1E3, ECM-1D4, ECM-1C5, ECM-1D6, ECM-1C7, ECM-1G7, ECM-1A10, ECM-1C11, ECM-1G11 and ECM-1C12 were selected for sequence determination. This should give a good representation of the clones present in the elution of the different selection outputs.

FIG. 2 shows the sequence alignment of the clones that were picked from the selection outputs. There is a diversity of around 8 different VHH sequences, which were derived from two families (KEREF and KQREL) within the sequences.

### Cloning and Production of VHH Selected on CmeC

From all the clones that were sequenced, ECM-1C1, ECM-1F2, ECM-1E3, ECM-1D4, ECM-1C5, ECM-1D6, ECM-1G7 and ECM-1C11 were subcloned into an expression vector. VHH genes were cut out with SfiI and Eco91I from phagemid pUR8100 in to pMEK222 with the same sites. pMEK222 adds a FLAG and His-tag at the C-terminus of the VHH.

The VHHs were produced as described below:

Pre-cultures were prepared by growing the bacteria containing the plasmids containing the selected VHH in 8 ml 2×YT medium supplemented with 2% glucose and 100 μg/ml ampicillin overnight at 37° C. The pre-cultures were diluted into 800 ml fresh 2×YT that was pre-warmed at 37° C. and supplemented with 100 μg/ml ampicillin and 0.1% glucose. The bacteria were grown for 2 hours at 37° C. before induction of the VHH expression with 1 mM IPTG. The VHHs were expressed for 4 hours at 37° C. and bacteria were harvested by centrifugation. Bacteria pellets were resuspended into 30 ml PBS and frozen at −20° C.

### Purification and Analysis of the VHH

Frozen bacteria were thawed at room temperature and centrifugated to separate cell debris and soluble fraction, which contains the VHH. VHH were purified from the soluble fraction using affinity of the His-tag to sepharose charged Cobalt (TALON beads). Bound VHH were eluted with 150 mM imidazole and dialyzed against PBS. The protein concentration was measured using absorption at 280 nm and corrected according the molar extinction coefficient and the molecular weight of the different VHH.

TABLE 2
Calculation of the VHH concentration based on absorption
(A280) and the correction factor (CF).

| | | | | | | | Production |
|---|---|---|---|---|---|---|---|
| | | | | | | Conc. | level |
| VHH | Vector | Tag | CF | MW | A280 | (mg/ml) | (mg/L) |
| ECM-1C1 | pMEK222 | FLAG-His | 1.361 | 15839 | 10.576 | 7.772 | 11.657 |
| ECM-1F2 | pMEK222 | FLAG-His | 1.278 | 15702 | 1.972 | 1.543 | 2.315 |
| ECM-1E3 | pMEK222 | FLAG-His | 1.372 | 15708 | 2.516 | 1.834 | 2.750 |
| ECM-1D4 | pMEK222 | FLAG-His | 1.208 | 15379 | 1.355 | 1.122 | 0.841 |
| ECM-1C5 | pMEK222 | FLAG-His | 1.712 | 15799 | 4.558 | 2.662 | 3.993 |
| ECM-1D6 | pMEK222 | FLAG-His | 1.376 | 15662 | 1.512 | 1.099 | 1.648 |
| ECM-1G7 | pMEK222 | FLAG-His | 1.392 | 15490 | 0.674 | 0.484 | 0.727 |
| ECM-1C11 | pMEK222 | FLAG-His | 1.788 | 15134 | 1.354 | 0.757 | 1.136 |

SDS PAGE showing analysis of purified VHH was conducted. All VHHs seem to be pure, ECM-1C1 and ECM-1F2 seem to show an extra band, which probably indicates some minor E. coli proteins that have been purified together with the VHH. This has been observed before and normally does not affect the following assays.

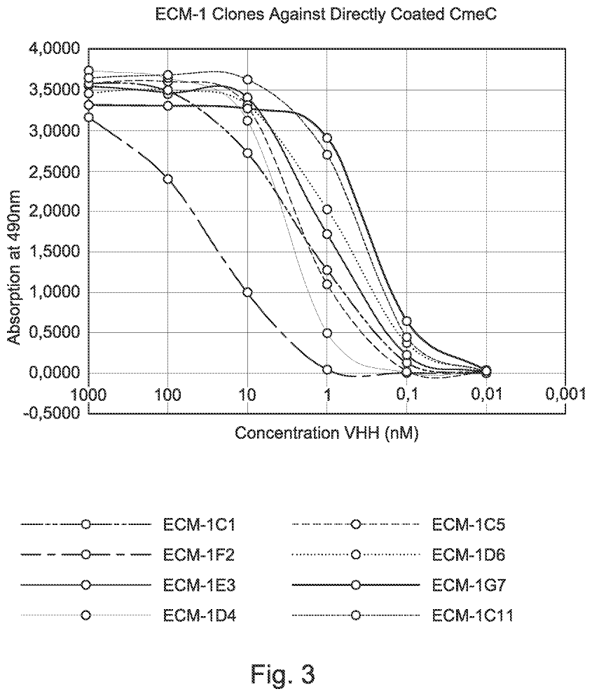

The binding of purified VHH was analyzed by ELISA on immobilized CmeC. A MaxiSorp plate was coated with 200 ng/well antigen overnight at 4° C. in PBS. After blocking the wells with 4% MPBS, a serial dilution of the VHHs was added to the coated wells and incubated for 1 hour at room temperature. After washing unbound VHHs, bound VHHs were detected using an anti-flag antibody and an anti-mouse coupled to a peroxidase. Binding was quantified by measuring colorimetric reaction of OPD+H₂O₂ at 490 nm (FIG. 3). The binding of the VHHs against CmeC is shown in FIG. 3. ECM-1F2 has a more moderate affinity. ECM-1C1, ECM-1E3, ECM-1D4 and ECM-1C5 have a low nanomolar affinity and ECM-1D6, ECM-1G7 and ECM-1C11 show a very high affinity with an apparent subnanomolar affinity to CmeC.

VHH sequences for some exemplary Campylobacter nanobodies are provided. The poly His tag is underlined.

### Nanobodies Directed Against CmeC Target

The first sequence for each provides the nanobody with the cMyc-His tag sequence underlined. The second sequence provides the VHH sequence without a tag and corresponding to a standard VHH domain sequence.

>CmeC-ECM-IC11
(SEQ ID NO: 66)
EVQLVESGGGLVQPGGSLRLSCAASGFTFSDQPMGWYRQAPGQGR
EWIAFISSGSGGTTDYKNSVKGRFTISRDNAKNIVYLQMNSLKPD
DTARYYCNFRIDNNYWGQGTQVTVSSAAASGSLEQKLISEEDLNG
AAHHHHHHGAA
(SEQ ID NO: 78)
EVQLVESGGGLVQPGGSLRLSCAASGFTFSDQPMGWYRQAPGQGR
EWIAFISSGSGGTTDYKNSVKGRFTISRDNAKNIVYLQMNSLKPD
DTARYYCNFRIDNNYWGQGTQVTVSSAAASGSL
>CmeC-ECM-IG7
(SEQ ID NO: 67)
EVQLVESGGDLVQDGGSLRLSCAASGGDFRNPVTGWYRQAPGKQR
ELVGLITSDGRTNYGDSVMGRFTISMDTAKNTMYLQMNSLKPEDS
AVYYCAASLLYGGLOFSTNIWGQGTQVTVSSAAASGSLEQKLISE
EDLNGAAHHHHHHGAA
(SEQ ID NO: 78)
EVQLVESGGDLVQDGGSLRLSCAASGGDFRNPVTGWYRQAPGKQR
ELVGLITSDGRTNYGDSVMGRFTISMDTAKNTMYLQMNSLKPEDS
AVYYCAASLLYGGLQFSTNIWGQGTQVTVSSAAASGSL
>CmeC-ECM-1D6
(SEQ ID NO: 1)
EVQLVESGGGLVQAGGSLRLSCAASGGTFSRYMMGWFRQAPGKER
EFVASIARSGGTYFADSVKGRLTATRDDAKNTMHLQMNSLKPEDT
AVYYCAAGAGGSYPALLDFEYLVWGQGTQVTVSSAAASGSLEQKL
ISEEDLNGAAHHHHHHGAA
(SEQ ID NO: 80)
EVQLVESGGGLVQAGGSLRLSCAASGGTFSRYMMGWFRQAPGKER
EFVASIARSGGTYFADSVKGRLTATRDDAKNTMHLQMNSLKPEDT
AVYYCAAGAGGSYPALLDFEYLVWGQGTQVTVSSAAASGSL

### Example 3

### Selection and Screening of VHHs Against CadF

Nanobodies (VHH antibodies) against CadF were isolated, selected and screened using the same and corresponding protocol and steps described above in Example 2, with CadF protein as antigen.

### Sequence Analysis of VHH

Based on the ELISA results and the HinfI pattern (data not shown), clones ECF-1A1, ECF-1D1, ECF-1E1, ECF-1E2, ECF-1E5, ECF-1F5, ECF-1B7, ECF-1G8, ECF-1H8, ECF-1C9, ECF-1B10, ECF-1D10, ECF-1F10 and ECF-1A12 were selected for sequence determination. This should give a good representation of the clones present in the elution of the different selection outputs,

FIG. 4 shows the sequence alignment of the clones that were picked from the selection outputs. There is a diversity of around 9 different VHH sequences based on the epitope families, which were derived from two different germline families (KEREF and KGLEW) within the sequences.

From all the clones that were sequenced, ECF-1D1, ECF-1E1, ECF-1F5, ECF-1B7, ECF-1G8, ECF-1H8, ECF-1B10, ECF-1D10 and ECF-1F10 were subcloned into an expression vector. VHH genes were cut out with SfiI and Eco91I from phagemid pUR8100 in to pMEK222 with the same sites. pMEK222 adds a FLAG and His-tag at the C-terminus of the VHH. ECF-1D1 failed to be recloned correctly and was therefore excluded.

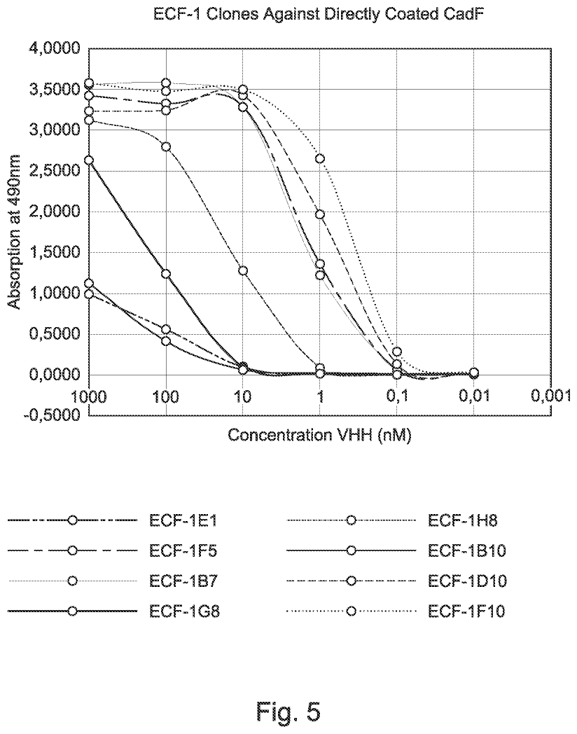

The binding of purified VHH was analyzed by ELISA on immobilized CadF. A MaxiSorp plate was coated with 200 ng/well antigen overnight at 4° C. in PBS. After blocking the wells with 4% MPBS, a serial dilution of the VHH was added to the coated wells and incubated for 1 hour at room temperature. After washing unbound VHH, bound VHH were detected using a rabbit-anti-VHH antibody and an anti-rabbit coupled to a peroxidase. Binding was quantified by measuring colorimetric reaction of OPD+H₂O₂ at 490 nm (FIG. 5).

The binding of the VHH against CadF is shown in FIG. 5. ECF-1E1, ECF-1G8 and ECF-1B10 do not reach a plateau phase, and show a more moderate affinity. ECF-1H8 also has a more moderate affinity. ECF-1F5 and ECF-1B7 have a low nanomolar affinity. ECF-1D10 and ECF-1F10 show an apparent subnanomolar affinity to CadF. ECF-1F5, ECF-1B7, ECF-1D10 and ECF-1F10 were considered as lead clones for follow up experiments.

The sequences of clones 1F10 and 1D10 are again provided below. His tag is underlined.

### Nanobodies Directed Against CadF Target

The first sequence for each provides the nanobody with the cMyc-His tag sequence underlined. The second sequence provides the VHH sequence without a tag and corresponding to a standard VHH domain sequence.

>CadF-ECF-IF10
(SEQ ID NO: 69)
EVQLVESGGGSVQAGGSLRLSCTASIRAGNRYAMGWFRQAPGKER
EFVAGISWSGGNTYHADSVNGRFTISRDNAKNTLYLTMNSLKSED
TAVYYCAASWKPLTFRGDDYTYWGQGTQVTVSSAAASGSLEQKLI
SEEDLNGAAHHHHHHGAA
(SEQ ID NO: 82)
EVQLVESGGGSVQAGGSLRLSCTASIRAGNRYAMGWFRQAPGKER
EFVAGISWSGGNTYHADSVNGRFTISRDNAKNTLYLTMNSLKSED
TAVYYCAASWKPLTFRGDDYTYWGQGTQVTVSSAAASGSL
>CadF-ECF-ID10
(SEQ ID NO: 68)
EVQLVESGGGLVQPGGSLRLSCAASGFTLDDYAVGWFRQAPGKER
EWLSGISSRDGSTVYADSVKGRFTISRDNAKNTYYLOMNSLKPED
TAVYYCAAAVGYILTRVKSHYNDWSQGTQVTVSSAAASGSLEQKL
ISEEDLNGAAHHHHHHGAA
(SEQ ID NO: 81)
EVQLVESGGGLVQPGGSLRLSCAASGFTLDDYAVGWFRQAPGKER
EWLSGISSRDGSTVYADSVKGRFTISRDNAKNTYYLQMNSLKPED
TAVYYCAAAVGYILTRVKSHYNDWSQGTQVTVSSAAASGSL

### Example 4

### Selection and Screening of VHHs Against CfrA

Nanobodies (VHH antibodies) against CfrA were isolated, selected and screened using the same and corresponding protocol and steps described above in Example 2, with CfrA protein as antigen.

### Sequence Analysis of VHH

Based on the ELISA results, clones ECA-1B6, ECA-1D7, ECA-1G7, ECA-1B8, ECA-1C8, ECA-1D9, ECA-1E9, ECA-1D10, ECA-1F10, ECA-1B11, ECA-1B12 and ECA-1E12 were selected for sequence determination. This should give a good representation of the clones present in the elution of the different selection outputs.

FIG. 6 shows the sequence alignment of the clones that were picked from the selection outputs. There is a diversity of around 6 different VHH sequences based on the epitope families, which were derived from three different germline families (KEREF, KQREL and KEREG) within the sequences.

### Cloning and Production of VHH Selected on CfrA

From all the clones that were sequenced, ECA-1G7, ECA-1C8, ECA-1D10, ECA-1B11, ECA-1B12 and ECA-1E12 were subcloned into an expression vector. VHH genes were cut out with SfiI and Eco91I from phagemid pUR8100 in to pMEK222 with the same sites. pMEK222 adds a FLAG and His-tag at the C-terminus of the VHH. The VHH were produced as described below:

Pre-cultures were prepared by growing the bacteria containing the plasmids containing the selected VHH in 8 ml 2×YT medium supplemented with 2% glucose and 100 μg/ml ampicillin overnight at 37° C. The pre-cultures were diluted into 800 ml fresh 2×YT that was pre-warmed at 37° C. and supplemented with 100 μg/ml ampicillin and 0.1% glucose. The bacteria were grown for 2 hours at 37° C. before induction of the VHH expression with 1 mM IPTG. The VHH were expressed for 4 hours at 37° C. and bacteria were harvested by centrifugation. Bacteria pellets were resuspended into 30 ml PBS and frozen at −20° C.

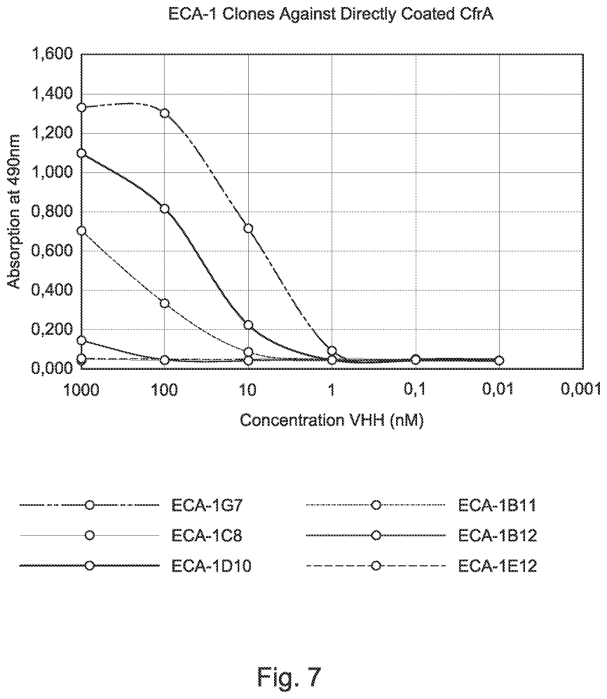

The binding of purified VHH was analyzed by ELISA on immobilized CfrA. A MaxiSorp plate was coated with 200 ng/well antigen overnight at 4° C. in PBS. After blocking the wells with 4% MPBS, a serial dilution of the VHH was added to the coated wells and incubated for 1 hour at room temperature. After washing unbound VHH, bound VHH were detected using an anti-flag antibody and an anti-mouse coupled to a peroxidase. Binding was quantified by measuring colorimetric reaction of OPD+H₂O₂ at 490 nm (FIG. 7).

The binding of the VHH against CfrA is shown in FIG. 7. Unfortunately, ECA-1C8, ECA-B12 and ECA-1E12 do not seem to show binding to CfrA at the tested concentrations of VHH. ECA-1D10 and ECA-1B11 have a more moderate affinity. ECA-1G7 has an apparent low nanomolar affinity to CfrA and should be considered as the lead VHH.

VHH sequences for some exemplary Campylobacter nanobodies are provided.

### Nanobodies Directed Against CfrA Target

The first sequence for each provides the nanobody with the cMyc-His tag sequence underlined. The second sequence provides the VHH sequence without a tag and corresponding to a standard VHH domain sequence.

>CfrA-ECA-1G7
(SEQ ID NO: 29)
EVQLVESGGGLVQPGGSLRLSCTASGSSLDYYAIGWFRQAPGKER
EGVSCISSRDGRIYWADSVEGRFTISRDNGKNTVYLQMNSLKPED
TAVYYCATSRWSFCPSDWSPVPSPFGSWGQGTQVTVSSAAASGSL
EQKLISEEDLNGAAHHHHHHGAA
(SEQ ID NO: 91)
EVQLVESGGGLVQPGGSLRLSCTASGSSLDYYAIGWFRQAPGKER
EGVSCISSRDGRIYWADSVEGRFTISRDNGKNTVYLQMNSLKPED
TAVYYCATSRWSFCPSDWSPVPSPFGSWGQGTQVTVSSAAASGSL

### Example 5

### Selection and Screening of VHHs Against CjaD

Nanobodies (VHH antibodies) against CjaD were isolated, selected and screened using the same and corresponding protocol and steps described above in Example 2, with CjaD protein as antigen.

### Sequence Analysis of VHHs

Based on the ELISA results and the HinfI pattern, clones ECD-1F1, ECD-1C3, ECD-1D3, ECD-1E4, ECD-1H4, ECD-1A7, ECD-1B7, ECD-1E7, ECD-1H7, ECD-1A8, ECD-1D9, ECD-1F10, ECD-1A11, ECD-1D11 and ECD-1E12 were selected for sequence determination. This should give a good representation of the clones present in the elution of the different selection outputs.

FIG. 8 shows the sequence alignment of the clones that were picked from the selection outputs. There is a diversity of around 12 different VHH sequences based on the epitope families, which were derived from three different germline families (KEREF, KQREL and KGLEW) within the sequences.

### Cloning and Production of VHH Selected on CjaD

From all the clones that were sequenced, ECD-1F1, ECD-1D3, ECD-1E4, ECD-1H4, ECD-1A7, ECD-1E7, ECD-1H7, ECD-1A8, ECD-1D9, ECD-1F10, ECD-1A11 and ECD-1D11 were subcloned into an expression vector. VHH genes were cut out with SfiI and Eco911 from phagemid pUR8100 in to pMEK222 with the same sites. pMEK222 adds a FLAG and His-tag at the C-terminus of the VHH. ECD-1E7 failed to be recloned correctly and is therefore excluded.

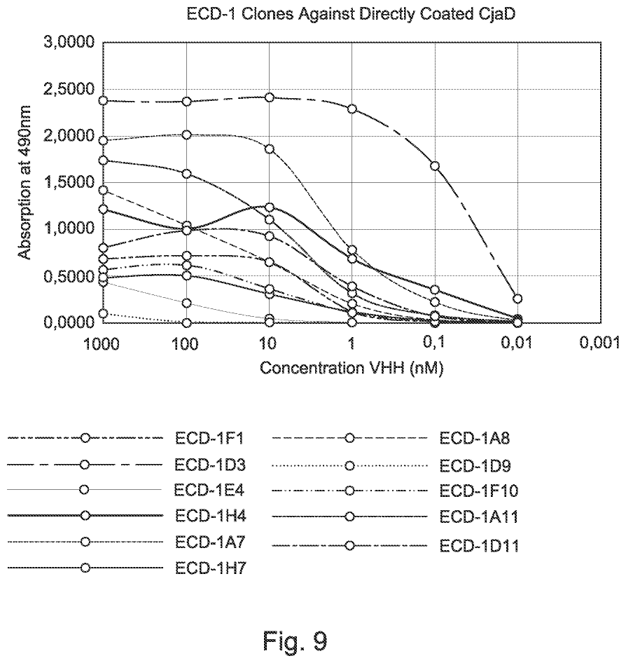

The binding of purified VHH was analyzed by ELISA on immobilized CjaD. A MaxiSorp plate was coated with 200 ng/well antigen overnight at 4° C. in PBS. After blocking the wells with 4% MPBS, a serial dilution of the VHHs was added to the coated wells and incubated for 1 hour at room temperature. After washing unbound VHH, bound VHH were detected using an anti-flag antibody and an anti-mouse coupled to a peroxidase. Binding was quantified by measuring colorimetric reaction of OPD+H₂O₂ at 490 nm (FIG. 9).

The binding of the VHH against CjaD is shown in FIG. 9. ECD-1D9 shows no clear binding on CjaD. ECD-1E4 and ECD-1A8 do not reach a plateau phase, and show a more moderate affinity. ECD-1F1, ECD-1A7, ECD-1H7, ECD-1F10, ECD-1A11 and ECD-1D11 have a low nanomolar affinity. ECD-1H4 show an apparent subnanomolar affinity to CjaD. ECD-1D3 shows a binding curve which is very rarely observed. This clone apparently has an affinity of around 60 pM, which is one of the best single domain antibodies ever selected. The actual binding affinity should be confirmed with methods like SPR. Many of the clones also give a different pattern (such as different Bmax, etc.) which is an indication that different epitopes on CjaD have been targeted. ECD-1D9 and ECD-1A11 have a very low production level so do not seem to be the best choice as lead clone.

VHH sequences for some exemplary Campylobacter nanobodies are provided.

### Nanobodies Directed Against CjaD Target

The first sequence for each provides the nanobody with the cMyc-His tag sequence underlined. The second sequence provides the VHH sequence without a tag and corresponding to a standard VHH domain sequence.

>CjaD-ECD-1D3
(SEQ ID NO: 49)
EVQLVESGGGLVQTGGSLRLSCATSGFTFEYSAIAWFRQAPGKGR
EGVSCISNRDGTTVYADSVKGRFTISSDNAKNTVYLQMNSLTPED
TGVYYCAADAGVYTADYCRDSRYDPVSKDAWGKGTLVTVSSAAAS
GSLEQKLISEEDLNGAAHHHHHHGAA
(SEQ ID NO: 84)
EVQLVESGGGLVQTGGSLRLSCATSGFTFEYSAIAWFRQAPGKGR
EGVSCISNRDGTTVYADSVKGRFTISSDNAKNTVYLQMNSLTPED
TGVYYCAADAGVYTADYCRDSRYDPVSKDAWGKGTLVTVSSAAAS
GSL
>CjaD-ECD-1A7
(SEQ ID NO: 53)
EVQLVESGGGLVQAGGSLRLSCATSGFTPGDYAIGWFRQAPGKAR
EGVSCLSTRDGTTYYADSVKGRFTISTDNAQNTVYLQMNSLKPED
TAVYYCAGEQISFRAVYYCTEYEPVYWSQGTQVTVSSAAASGSLE
QKLISEEDLNGAAHHHHHHGAA
(SEQ ID NO: 85)
EVQLVESGGGLVQAGGSLRLSCATSGFTPGDYAIGWFRQAPGKAR
EGVSCLSTRDGTTYYADSVKGRFTISTDNAQNTVYLQMNSLKPED
TAVYYCAGEQISFRAVYYCTEYEPVYWSQGTQVTVSSAAASGSL

### Example 6

### Selection and Screening of VHHs Against Flagella

Nanobodies (VHH antibodies) against flagella were isolated, selected and screened using the same and corresponding protocol and steps described above in Example 2, with C. jejuni flagella as antigen.

### Sequence Analysis of VHHs

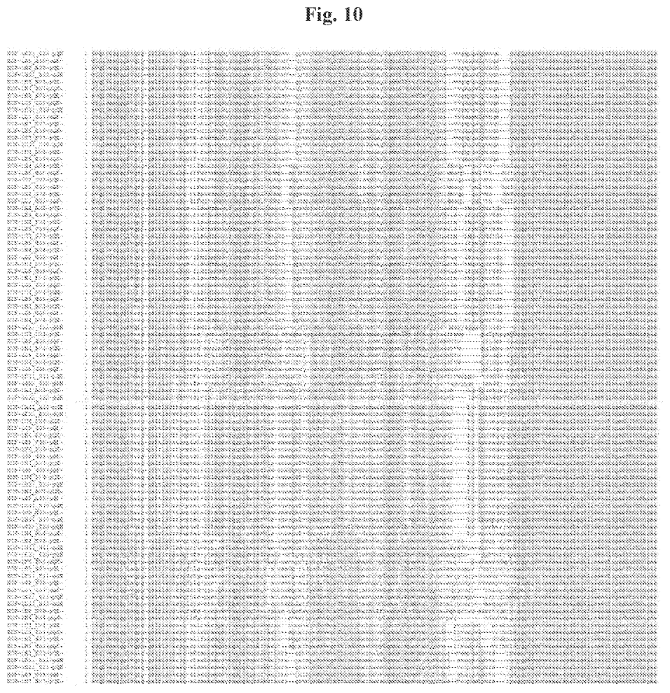

To determine the diversity of the VHH binding to Cj flagella in peri-ELISA, the entire master plates ECF-1 continuing single clones from the outputs of the selections was sequenced and the amino acid sequences were aligned. These sequences are shown in FIG. 10. Based on these sequences and the signals in peri-ELISA, a total of 10 clones were then selected and these were recloned into the E. coli production vector pMEK222. This also provides the VHH with a C-terminal FLAG-His tag. The amino acid sequences of these clones-amino acid sequences of Cj flagella-binding VHH in pMEK222 are provided in FIG. 11.

### Production, Purification and Dose Response ELISA

The 10 candidate VHHs were produced in TG1 E. coli and were purified by immobilized metal affinity chromatography (IMAC) using Cobalt beads and the C-terminal His tag. Integrity of VHH was confirmed by SDS-PAGE and PageBlue staining. Next the apparent binding affinity of the purified VHH's was determined in an ELISA. A serial dilution of the VHHs was added to the coated wells and incubated for 1 hour at room temperature starting at 1000 nM. Bound VHH's were detected with rabbit-anti-VHH (QE19), followed by donkey-anti-rabbit HRP and made visible with OPD. Estimated apparent binding affinities are summarized in Table 3.

TABLE 3
Estimated apparent binding affinities
of ECF VHHs to C. jejuni flagella

| | | Apparent | |
|---|---|---|---|
| | Relative | affinity | |
| VHH | Bmax | (nM) | Notes |
| ECF-1H3 | 1.5 | >400 | NP |
| ECF-1C4 | 3 | 4 | |
| ECF-1E4 | 0.8 | >100 | NP |
| ECF-1F4 | 2.5 | 0.6 | |
| ECF-1B6 | 2.5 | 5 | |
| ECF-1H7 | 1.4 | >400 | NP |
| ECF-1F8 | 2 | >500 | NP |
| ECF-1G8 | 2.5 | 7 | |
| ECF-1D9 | 3 | 9 | |
| ECF-1A10 | 1.8 | >500 | NP |

NP: No plateau fase

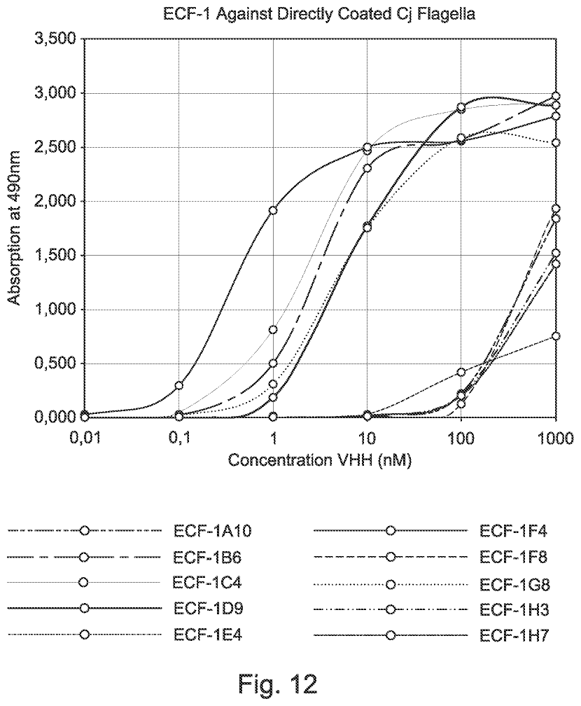

FIG. 12 shows binding of purified VHH to immobilized Cj flagella. Bound VHH were detected using rabbit anti-VHH (Cat #QE19), followed by donkey-anti-rabbit HRP and OPD as substrate.

VHHs binding to Cj flagella were generated. Immunizations were performed in two llamas (SNL190 and SNL191), which resulted in a moderate to good immune responses. RNA extracted from the peripheral blood lymphocytes was of good quality and the generated VHH libraries were of good sizes and insert ratio. Phages produced from these libraries were used in two round of selection on immobilized Cj flagella. High outputs with a concentration dependent enrichment were observed. Master plate ECF-1 was picked form the output of these selections and periplasmic extracts containing monoclonal VHH were produced. This periplasmic extracts were used to screen for Cj-flagella binding VHH in ELISA. Sequence analysis of the master plate resulted in the selection of 10 candidate VHH, which were assessed for apparent binding affinities on Cj flagella. Of the selected VHH, 5 VHH were able to bind to Cj flagella with binding affinities <10 nM.

VHH sequences for some exemplary Campylobacter nanobodies are provided.

### Sequences of Selected Nanobodies Directed Against Flagella Target

The first sequence for each provides the nanobody with the FLAG-His tag sequence underlined. The second sequence provides the VHH sequence without a tag and corresponding to a standard VHH domain sequence.

>Flagella-ECF-1C4
(SEQ ID NO: 73)
EVQLVESGGGLVQAGGSLRVSCAASGRAVHNYALNWFRQAPGKER
EFVAAISWTRRTYYANSVRGRFTISRDNNGNMVHLQMSNLKSEDT
ALYYCAAATTWPRLDGAEYWGQGTQVTVSSAAADYKDDDDKGAAH
HHHHHGAA
(SEQ ID NO: 86)
EVQLVESGGGLVQAGGSLRVSCAASGRAVHNYALNWFRQAPGKER
EFVAAISWTRRTYYANSVRGRFTISRDNNGNMVHLQMSNLKSEDT
ALYYCAAATTWPRLDGAEYWGQGTQVTVSS
>Flagella-ECF-1F4
(SEQ ID NO: 74)
EVQLVESGGGLVQPGGSLRLSCEAAGSILRVNSMGWYRQAPGKQR
DLVAIITSNNDAKYSDSVKGRFTISRDNAKNTVYLQMSSLKPDDT
AVYYCNAYVETAGWIPTTHNLWGQGTQVTVSSAAADYKDDDDKGA
AHHHHHHGAA
(SEQ ID NO: 87)
EVQLVESGGGLVQPGGSLRLSCEAAGSILRVNSMGWYRQAPGKQR
DLVAIITSNNDAKYSDSVKGRFTISRDNAKNTVYLQMSSLKPDDT
AVYYCNAYVETAGWIPTTHNLWGQGTQVTVSSAAA
>Flagella-ECF-1B6
(SEQ ID NO: 75)
EVQLVESGGGLVQSGGSLRLSCAASGRIFSITNMGWYRQAPGEQR
ELVADMPSGGSTNYADSVKGRFTISRDIAKNTVYLQMNSLKPEDT
AVYYCNLKMSQPGWLVTNHNFWGQGTQVTVSSAAADYKDDDDKGA
AHHHHHHGAA
(SEQ ID NO: 88)
EVQLVESGGGLVQSGGSLRLSCAASGRIFSITNMGWYRQAPGEQR
ELVADMPSGGSTNYADSVKGRFTISRDIAKNTVYLQMNSLKPEDT
AVYYCNLKMSQPGWLVTNHNFWGQGTQVTVSSAAA
>Flagella-ECF-1G8
(SEQ ID NO: 76)
EVQLVESGGGLVEAGGSLTLSCTTSEPTSLLNLMGWWRQGPGKER
EFVASSNWSGKLVDYADGVEGRFTVIRNEDENAISLQMNSLTVED
TAIYYCAASRLGLPRSAQAYQYWGQGTQVTVSSAAADYKDDDDKG
AAHHHHHHGAA
(SEQ ID NO: 89)
EVQLVESGGGLVEAGGSLTLSCTTSEPTSLLNLMGWWRQGPGKER
EFVASSNWSGKLVDYADGVEGRFTVIRNEDENAISLQMNSLTVED
TAIYYCAASRLGLPRSAQAYQYWGQGTQVTVSSAAA
>Flagella-ECF-1D9
(SEQ ID NO: 77)
EVQLVESGGGLVQPGGSLRLSCATSGFAFDNYCMYWVRQAPGKGL
EWISGITNGGSFSYYADSVKGRFTISRDNAKNTLFLEMNSLKSDD
TAVYYCAIHGHGCTWDSLRTTSGPRYRGQGTQVTVSSAAADYKDD
DDKGAAHHHHHHGAA
(SEQ ID NO: 90)
EVQLVESGGGLVQPGGSLRLSCATSGFAFDNYCMYWVRQAPGKGL
EWISGITNGGSFSYYADSVKGRFTISRDNAKNTLFLEMNSLKSDD
TAVYYCAIHGHGCTWDSLRTTSGPRYRGQGTQVTVSSAAA

A comparison and alignment of all of the selected nanobodies—directed against CfrA, CmeC, CjaD, CadF and flagella Campylobacter proteins—is provided in FIG. 13. FIG. 13 also provides a consensus nanobody amino acid sequence. Nanobody(ies) and nanobody variants comprising the unique nanobody sequence and particularly comprising CDR domain sequences CDR1, CDR2 and CDR3 as set out in each applicable corresponding antibody are contemplated.

### Example 7

### Evaluation of Nanobodies In Vivo

Poultry (chicken broilers) are utilized to evaluate the nanobodies individually and in various combinations in a challenge study. Animals are administered nanobody(ies) orally by administering bacteria expressing one or more nanobodies on Day 1 of the study. Animals are challenged with Campylobacter on Day 2 and are then evaluated by necropsy on Day 7.

### Example 8

### Pilot Efficacy Study: Efficacy Evaluation of Multivalent Anti-Campylobacter Vaccine Candidates in Broilers Challenged with Campylobacter jejuni that are Housed in Battery Cages

This study was conducted in battery cages located in a temperature-controlled, enclosed, BSL-2 facility. There were 30 treatment groups with 5 to 10 numbers of birds per treatment group (see Table 1). Treatment was assigned to pen (5 birds per pen) within a battery cage system based on biosecurity principles used to prevent cross-contamination.

Day 0 is the day of receipt of the procured birds. On Day 1 following humane euthanasia of TG1 birds, necropsy with 1 of the paired ceca was collected aseptically for assessment of Campylobacter-free status by bacterial culture of cecal contents. Two pens represented the entire treatment group designated as TG2. One pen represented the entire treatment group designated as TG3 through TG30. Placement of the battery cages for TG2 was located strategically away from both the ventilation inlet and exhaust areas. Birds in TG3 and TG4 were not inoculated with a vaccine on Day 1 but were treated (gavaged, 0.1 mL) with ciprofloxacin instead, Birds in TG5 and TG6 were inoculated (gavaged, 0.1 mL) with sterile water as a control product on Day 1 only. Birds in TG7 through 30 were inoculated (gavaged, 0.1 mL) with L. reuteri vectored multivalent anti-C. jejuni vaccine candidates on Day 1 only (see Table 1). Birds in TG1 and TG2 were not inoculated with anything (neither gavaged with water nor L. reuteri vectored multivalent anti-C. jejuni vaccine) on Day 1.

Birds in TG2 were challenged (gavaged, 0.1 mL) with a control solution (sterile Mueller Hinton broth) on Day 2 only. Birds in TG3 through 30 were challenged (gavaged, 0.1 mL) with 1×103 or 1×105 CFUs/mL of C. jejuni on Day 2 only. Birds in TG1 were already removed from the study on Day 1 and were not challenged with 1×103 or 1×105 CFUs/mL of C. jejuni. The volume of the oral gavage for C. jejuni challenge was 0.1 mL (100 μL) regardless of the CFUs/mL.

On Day 8 following humane euthanasia for TG2 through TG30, necropsy occurred with 1 of the paired ceca collected aseptically for culture of gut contents. Ceca received were weighed, have 1 mL of sterile PBS added, stomached, serially diluted, and plated in replicates (2 times) on Campylobacter selective media. The plates were incubated under microaerophilic conditions at 39 C for 24 to 48 hours. Then, CFU counts/g of cecal contents were determined, recorded in a Campylobacter jejuni Colonization and Enumeration Record.

Lactobacillus reuteri strains 3630 and 3632 are described and detailed as novel strains suitable as DFMs, including in combination, and also as suitable strains for genetic modification and as live delivery or production strains.

Lactobacillus reuteri strain 3632 was deposited on 19 Jun. 2020 according to the Budapest Treaty in the ATCC Patent Depository and assigned ATCC Patent Deposit Number PTA-126788. Lactobacillus reuteri strain 3630 was deposited on 19 Jun. 2020 in the ATCC Patent Depository and assigned ATCC Patent Deposit Number PTA-126787.

The L. reuteri strains 3630 and 3632 are described and detailed as probiotic strains in Probiotic Compositions Comprising Lactobacillus Reuteri Strains and Methods of Use PCT/US2020/016668 filed Feb. 4, 2020, published as WO 2020/163398 Aug. 13, 2020. Priority parent is 62/801,307 filed Feb. 5, 2019. Corresponding US publications are US 2022/0088094 published Mar. 24, 2022 and US 2022/0125860 published Apr. 28, 2022.

A live delivery system based on L. reuteri strain 3630 or 3632 is described and detailed in A Genetically Modified Lactobacillus and Uses Thereof PCT/US2020/016522 filed Feb. 4, 2020, published as WO 2020/163284 Aug. 13, 2020. Priority parent is 62/801,307 filed Feb. 5, 2019. This application describes native bacterial promoters, signal sequences suitable for expression and vectors and bacterial genome sites/genes for integration to generate stable modified strains.

L. reuteri Delivery of VHH Nanobodies:

Direct fed microbials (DFMs), often also called probiotics, are microorganisms which colonize the gastrointestinal tract of an animal and provide some beneficial effect to that animal. The microorganisms can be bacterial species, for example those from the genera Bacillus, Lactobacillus, Lactococcus, and Enterococcus. The microorganisms can also be yeast or even molds. The microorganisms can be provided to an animal orally or mucosally or, in the case of birds, provided to a fertilized egg, i.e. in ovo.

Lactobacillus reuteri strains 3630 and 3632 are described and detailed as novel strains suitable as DFMs, including in combination, and also as suitable strains for genetic modification and as live delivery or production strains.

The L. reuteri strains 3630 and 3632 are described and detailed as probiotic strains in Probiotic Compositions Comprising Lactobacillus Reuteri Strains and Methods of Use-PCT/US2020/016668 filed Feb. 4, 2020, published as WO 2020/163398 Aug. 13, 2020. Priority parent is 62/801,307 filed Feb. 5, 2019. Corresponding US publications are US 2022/0088094 published Mar. 24, 2022 and US 2022/0125860 published Apr. 28, 2022. The content of the aforementioned applications are hereby incorporated by reference in the instant application.

A live delivery system based on L. reuteri strain 3630 or 3632 is described and detailed in A Genetically Modified Lactobacillus and Uses Thereof-PCT/US2020/016522 filed Feb. 4, 2020, published as WO 2020/163284 Aug. 13, 2020. Priority parent is 62/801,307 filed Feb. 5, 2019. The content of the aforementioned applications are hereby incorporated by reference in the instant application.

Recombinant Lactobacillus (L. reuteri strain 3630 and L. reuteri strain 3632) delivering nanobodies directed against Clostridium perfringes NetB and alpha toxin have been described and shown to confer protection against necrotic enteritis in poultry (Gangaiah D et al MicrobiologyOpen 2022; 11: e1270, doi.org/10.1002/mbo3.1270).

To evaluate the efficacy of L. reuteri vectored multivalent anti-Campylobacter jejuni vaccines (Table 1) to reduce (or eliminate) C. jejuni in broilers housed in battery cages. Cecal colonization (infection) was assessed by enumeration of CFU/g of ceca on Day 8. The results are presented in Table 2. Results are expressed in log 10 CFU/g ceca. The challenge models were assessed by comparing TG2 (Environmental control) with TG5 (1×103 CFU/mL) and TG6 (1×105 CFU/mL) as shown in Table 3; there was a significant difference (p=0.001) in CFU/g ceca between the environmental control group and challenge groups indicating that the inoculum successfully induced significant colonization. The performance of twelve multivalent anti-Campylobacter vaccine candidates were assessed for their ability to either reduce or eliminate C. jejuni colonization in the cecum. Each candidate was challenged with one of the two concentrations of inoculum. The TGs treated with vaccine candidates did not show any statistical difference in comparison to the disease groups TG5 and TG6. However, vaccine candidates TG19, TG21, TG27, and TG29 showed significant biological effect by reducing C. jejuni colonization by one-half (0.5) log 10 count or greater as shown in Table 3. Based on these results it can be inferred that (at minimum) TG19, TG21, TG27, and TG29 had clinical significance and can be considered as candidates for future studies.

TABLE 1
L. reuteri strains delivering different VHH clones
targeting different C. jejuni virulence factors.

| Unique ID | Species | Strain | VHH | Target |
|---|---|---|---|---|
| CJ01 | Lactobacillus reuteri | 3632 | ECM-1C11 | CmeC |
| CJ02 | Lactobacillus reuteri | 3632 | ECM-1G7 | CmeC |
| CJ03 | Lactobacillus reuteri | 3632 | ECF-1F10 | CadF |
| CJ04 | Lactobacillus reuteri | 3632 | ECF-1D10 | CadF |
| CJ05 | Lactobacillus reuteri | 3632 | ECA-1G7 | CfrA |
| CJ06 | Lactobacillus reuteri | 3632 | ECD-1D3 | CjaD |
| CJ07 | Lactobacillus reuteri | 3632 | ECD-1A7 | CjaD |
| CJ08 | Lactobacillus reuteri | 3632 | FlagV1M | Flagella |
| CJ09 | Lactobacillus reuteri | 3632 | FlagV6M | Flagella |
| CJ10 | Lactobacillus reuteri | 3632 | ECF-1C4 | Flagella |
| CJ11 | Lactobacillus reuteri | 3632 | ECF-1F4 | Flagella |
| CJ12 | Lactobacillus reuteri | 3632 | ECF-1B6 | Flagella |
| CJ13 | Lactobacillus reuteri | 3632 | ECF-1G8 | Flagella |
| CJ14 | Lactobacillus reuteri | 3632 | ECF-1D9 | Flagella |

TABLE 2
The Results of Ceca Enumeration in TGs expressed in CFUs/g.

| | | | C. [symbol] | | | | # Of Birds | | |
|---|---|---|---|---|---|---|---|---|---|
| | | | Challenge | | | | w. | [symbol] (CFU/g) | |
| | | # Of | on Day 2 | CFU/g | | | [symbol] | | Std. |
| Treatment Group | | Birds | (CFUs/mL) | Medium | Min | Max | TNC† | Mean | Dev. |
| TG1 | Cj-free [symbol] | 5 | None | −8 | −8 | −8 | 0 | −0.93 | — |
| | UPREC | | | | | | | | |
| TG2 | ENVI [symbol] | 10 | [symbol] | −8 | −8 | −8 | 0 | −0.91 | — |
| TG3 | CPFX [symbol] 1E3 | 5 | 1 × 10 [symbol] | 1.69E7 | 1418.2 | 7.08E7 | 0 | 6.3 | 2.0 |
| TG4 | CPFX [symbol] 1E5 | 5 | 1 × 10 [symbol] | [symbol] | 81077 | 541913 | 0 | 5.2 | 0.3 |
| TG5 | NCC 1E3 | 5 | 1 × 10 [symbol] | 2.75E7 | 492403 | −2.08E8 | 1 | 7.1 | 1.4 |
| TG6 | NCC 1E5 | 5 | 1 × 10 [symbol] | 5E6 | 62229 | 1.17E8 | 0 | 6.6 | 1.5 |
| TG7 | [symbol] | 5 | 1 × 10 [symbol] | 1.07E8 | 39000 | 1.61E8 | 0 | 7.4 | 1.6 |
| | CJ2 1E3 | | | | | | | | |
| TG8 | [symbol] | 5 | 1 × 10 [symbol] | 2.4E7 | 1.21E6 | 6.36E7 | 0 | 7.2 | 0.7 |
| | CJ2 1E5 | | | | | | | | |
| TG9 | [symbol] | 5 | 1 × 10 [symbol] | 6.51E7 | 4.16E7 | 2.03E8 | 0 | 7.9 | 0.3 |
| | CJ3 1E3 | | | | | | | | |
| TG10 | [symbol] | 5 | 1 × 10 [symbol] | 2.4E7 | 50697 | 6.03E7 | 0 | 7.0 | 1.3 |
| | CJ3 1E5 | | | | | | | | |
| TG11 | [symbol] | 5 | 1 × 10 [symbol] | 5.73E6 | 1.41E6 | >2.08E8 | 1 | 7.1 | 0.9 |
| | CJ5 1E3 | | | | | | | | |
| TG12 | [symbol] | 5 | 1 × 10 [symbol] | 5.08E7 | 3.42E6 | >2.08E8 | 1 | 7.7 | 0.8 |
| | CJ5 1E5 | | | | | | | | |
| TG13 | [symbol] | 5 | 1 × 10 [symbol] | 3.21E7 | 2E6 | 1.09E8 | 0 | 7.5 | 0.7 |
| | CJ6 1E3 | | | | | | | | |
| TG14 | [symbol] | 5 | 1 × 10 [symbol] | 3.72E7 | 815708 | 7.46E7 | 0 | 7.3 | 0.8 |
| | CJ6 1E5 | | | | | | | | |
| TG15 | [symbol] | 5 | 1 × 10 [symbol] | 6.6E6 | 639595 | 1.21E8 | 0 | 6.9 | 0.9 |
| | CJ8 1E3 | | | | | | | | |
| TG16 | [symbol] | 5 | 1 × 10 [symbol] | 2.36E7 | [symbol] | 6.32E7 | 0 | 6.6 | 1.3 |
| | CJ8 1E5 | | | | | | | | |
| TG17 | [symbol] | 5 | 1 × 10 [symbol] | 8.35E7 | 459375 | −2.08E8 | 1 | 7.2 | 1.3 |
| | CJ10 1E3 | | | | | | | | |
| TG18 | [symbol] | 5 | 1 × 10 [symbol] | 5.42E7 | 3.09E7 | 8.13E7 | 0 | 7.7 | 0.2 |
| | CJ10 1E5 | | | | | | | | |
| TG19 | [symbol] | 5 | 1 × 10 [symbol] | 153816 | 9242.6 | −2.08E8 | 1 | 5.7 | 1.9 |
| | CJ11 1E3 | | | | | | | | |
| TG20 | [symbol] | 5 | 1 × 10 [symbol] | 6.45E6 | 2.28E6 | 7.6E7 | 0 | 6.9 | 0.6 |
| | CJ11 1E5 | | | | | | | | |
| TG21 | [symbol] | 5 | 1 × 10 [symbol] | 595156 | 10084 | 8.91E7 | 0 | 5.9 | 1.7 |
| | CJ12 1E3 | | | | | | | | |
| TG22 | [symbol] | 5 | 1 × 10 [symbol] | 2.92E7 | 577778 | 1.38E8 | 0 | 7.1 | 1.0 |
| | CJ12 1E5 | | | | | | | | |
| TG23 | [symbol] | 5 | 1 × 10 [symbol] | 3.14E7 | 58018 | 1.11E8 | 0 | 6.8 | 1.4 |
| | CJ14 1E3 | | | | | | | | |
| TG24 | [symbol] | 5 | 1 × 10 [symbol] | 2.58E7 | 33483 | 5.71E7 | 0 | 6.8 | 1.3 |
| | CJ14 1E5 | | | | | | | | |
| TG25 | [symbol] | 5 | 1 × 10 [symbol] | 8.61E7 | 321082 | 1.64E8 | 0 | 7.2 | 1.2 |
| | CAP01 1E3 | | | | | | | | |
| TG26 | [symbol] | 5 | 1 × 10 [symbol] | 7.89E7 | 631269 | 9.44E7 | 0 | 7.3 | 1.0 |
| | CAP01 1E5 | | | | | | | | |
| TG27 | [symbol] | 5 | 1 × 10 [symbol] | 1.71E7 | 47816 | 4.64E7 | 0 | 6.5 | 1.3 |
| | CAP02 1E3 | | | | | | | | |
| TG28 | [symbol] | 5 | 1 × 10 [symbol] | 4.54E7 | 1.08E7 | >2.08E8 | 1 | 7.7 | 0.6 |
| | CAP02 1E5 | | | | | | | | |
| TG29 | [symbol] | 5 | 1 × 10 [symbol] | 353354 | 96.6 | >2.08E8 | 1 | 5.4 | 2.8 |
| | CAP03 1E3 | | | | | | | | |
| TG30 | [symbol] | 5 | 1 × 10 [symbol] | 5.63E7 | 4.36E6 | >2.08E8 | 1 | 7.7 | 0.8 |
| | CAP03 1E5 | | | | | | | | |

†TNC: too numerous to count: above upper limit of detection (ULOD) [symbol]
indicates data missing or illegible when filed

TABLE 3
Statistical analyses results of CFU counts.

| | | | Diff. to (P-value) | | |
|---|---|---|---|---|---|
| | Treatment Group | LS Mean +/− SE | TG2† | TG5 | TG6 |
| TG1 | Cj-free status UPREC | <1.2 | NA‡ | — | — |
| TG2 | ENVI. Ctrl | <1.2 | — | — | — |
| TG3 | CPFX Ctrl:1E3 | 6.27 +/− 0.88 | 5.37(0.004) | −0.87(0.443) | |
| TG4 | CPFX Ctrl:1E5 | 5.22 +/− 0.15 | 4.31(<.001) | — | −1.38(0.105) |
| TG5 | NCC:1E3 | 7.14 +/− 0.61 | 6.23(<.001) | — | — |
| TG6 | NCC:1E5 | 6.60 +/− 0.66 | 5.69(0.001) | — | — |
| TG7 | L. reuteri CJ2:1E3 | 7.39 +/− 0.70 | — | 0.25(0.793) | — |
| TG8 | L. reuteri CJ2:1E5 | 7.20 +/− 0.32 | — | — | 0.60(0.447) |
| TG9 | L. reuteri CJ3:1E3 | 7.92 +/− 0.14 | — | 0.79(0.269) | — |
| TG10 | L. reuteri CJ3:1E5 | 6.99 +/− 0.58 | — | — | 0.39(0.667) |
| TG11 | L. reuteri CJ5:1E3 | 7.05 +/− 0.42 | — | −0.08(0.913) | — |
| TG12 | L. reuteri CJ5:1E5 | 7.70 +/− 0.36 | — | — | 1.10(0.192) |
| TG13 | L. reuteri CJ6:1E3 | 7.45 +/− 0.32 | — | 0.32(0.659) | — |
| TG14 | L. reuteri CJ6:1E5 | 7.28 +/− 0.35 | — | — | 0.69(0.394) |
| TG15 | L. reuteri CJ8:1E3 | 6.93 +/− 0.38 | — | −0.20(0.785) | — |
| TG16 | L. reuteri CJ8:1E5 | 6.59 +/− 0.59 | — | — | 0.00(0.997) |
| TG17 | L. reuteri CJ10:1E3 | 7.22 +/− 0.60 | — | 0.08(0.927) | — |
| TG18 | L. reuteri CJ10:1E5 | 7.72 +/− 0.08 | — | — | 1.12(0.166) |
| TG19 | L. reuteri CJ11:1E3 | 5.66 +/− 0.83 | — | −1.48(0.191) | — |
| TG20 | L. reuteri CJ11:1E5 | 6.93 +/− 0.28 | — | — | 0.33(0.661) |
| TG21 | L. reuteri CJ12:1E3 | 5.88 +/− 0.74 | — | −1.26(0.228) | — |
| TG22 | L. reuteri CJ12:1E5 | 7.10 +/− 0.43 | — | — | 0.50(0.547) |
| TG23 | L. reuteri CJ14:1E3 | 6.79 +/− 0.62 | — | −0.35(0.698) | — |
| TG24 | L. reuteri CJ14:1E5 | 6.79 +/− 0.60 | — | — | 0.19(0.835) |
| TG25 | L. reuteri CAP01:1E3 | 7.21 +/− 0.55 | — | 0.08(0.927) | — |
| TG26 | L. reuteri CAP01:1E5 | 7.25 +/− 0.43 | — | — | 0.66(0.433) |
| TG27 | L. reuteri CAP02:1E3 | 6.46 +/− 0.60 | — | −0.67(0.453) | — |
| TG28 | L. reuteri CAP02:1E5 | 7.73 +/− 0.26 | — | — | 1.14(0.168) |
| TG29 | L. reuteri CAP03:1E3 | 5.41 +/− 1.26 | — | −1.72(0.266) | — |
| TG30 | L. reuteri CAP03:1E5 | 7.66 +/− 0.38 | — | — | 1.07(0.208) |

†Difference to log10(8) = 1.2.
‡NA: Not applicable

In conclusion, a total of twelve multivalent anti-Campylobacter vaccine candidates were assessed for their ability to either reduce or eliminate C. jejuni colonization in the cecum. None of the vaccinated groups demonstrated a statistically significant difference (reduction or elimination) in comparison to the disease group. However, four groups, including TG19, TG21, TG27, and TG29, were identified as lead candidates based on their results for successfully reducing the infection (CFU/g ceca) of C. jejuni by one-half (0.5) log 10 count or greater.

### Example 9

### Efficacy Evaluation of Multivalent Anti-Campylobacter Vaccines in Broilers Challenged With Campylobacter jejuni that are Housed in Floor Pens

The goal of this study was to evaluate the efficacy of L. reuteri vectored multivalent anti-C. jejuni vaccines selected from the pilot study to reduce (or eliminate) C. jejuni by one-half (0.5) log 10 count or greater from ceca samples collected from broilers housed in floor pens at 42 days of age. This study was conducted in floor pens of 5 separate isolation rooms located in a temperature-controlled, enclosed, BSL-2 facility. There were 5 treatment groups with 50 birds per treatment group upon study enrollment (see Table 4 and Table 5).

Treatment was assigned to room based on biosecurity principles used to prevent cross-contamination. Day-21 was the day of egg placement in the incubator. Day-3 was the day of in ovo inoculation with the test articles. Day 0 was the day of hatch and placement of 50 birds in the isolation room. Days 0, 14, and 35 were the days of treatment (gavaged, 0.1 mL) with the test articles. Day 15 was the day of challenge with 1×106 CFU/mL of the C. jejuni JB strain (gavaged, 0.1 mL). Day 35 was the day of cloacal swab sample collection from 10 birds per isolation room for C. jejuni enumeration. Day 42 was the day of necropsy for ceca sample collection from 30 birds per isolation room for C. jejuni enumeration.

TABLE 4
Treatment group details.

| | | | Test | Test Article | |
|---|---|---|---|---|---|
| Trt | | | Article | on Days 0, | Challenge |
| Grp | Description | n | on Day −3 | 14, and 35 | (CFU/mL) |
| 1 | Environmental | 50 | Sterile | Sterile | Sterile Mueller |
| | Control | | Marek's | Marek's | Hinton Broth |
| | | | Diluent | Diluent | (0) |
| 2 | Untreated, | 50 | Sterile | Sterile | C. jejuni JB |
| | Challenged | | Marek's | Marek's | strain (1 × 10⁶) |
| | Control | | Diluent | Diluent | |
| 3 | Lactobacillus | 50 | Assigned | Assigned | C. jejuni JB |
| | reuteri | | Vaccine | Vaccine | strain (1 × 10⁶) |
| | CJ08 | | | | |
| 4 | Lactobacillus | 50 | Assigned | Assigned | C. jejuni JB |
| | reuteri | | Vaccine | Vaccine | strain (1 × 10⁶) |
| | CJ14-CJ11-CJ12 | | | | |
| 5 | Lactobacillus | 50 | Assigned | Assigned | C. jejuni JB |
| | reuteri | | Vaccine | Vaccine | strain (1 × 10⁶) |
| | CJ2-CJ3-CJ5-CJ6 | | | | |

TABLE 5
Description of test candidates.

| GROUP | DESCRIPTION |
|---|---|
| T1 | Unchallenged control |
| T2 | Challenge control |
| T3 | L. reuteri 3632 delivering published anti-flagellar Nb |
| T4 | L. reuteri 3632 delivering antiflagellar Nbs CJ11, CJ12 and |
| | CJ14 in 1:1:1 ratio |
| T5 | L. reuteri 3632 delivering Nbs against CmeC, CjaD, CadF and |
| | CfrA combined in 1:1:1:1:1 ratio |

L. reuteri delivering anti-flagellar nanobodies significantly (TG4) (P<0.05) reduced Campylobacter load as determined by cloacal swabs on day 35 (FIG. 3). L. reuteri delivering nanobodies against Nbs against CmeC, CjaD, CadF and CfrA (TG5) numerically reduced Campylobacter load, however, it was not statistically significant (FIG. 3).

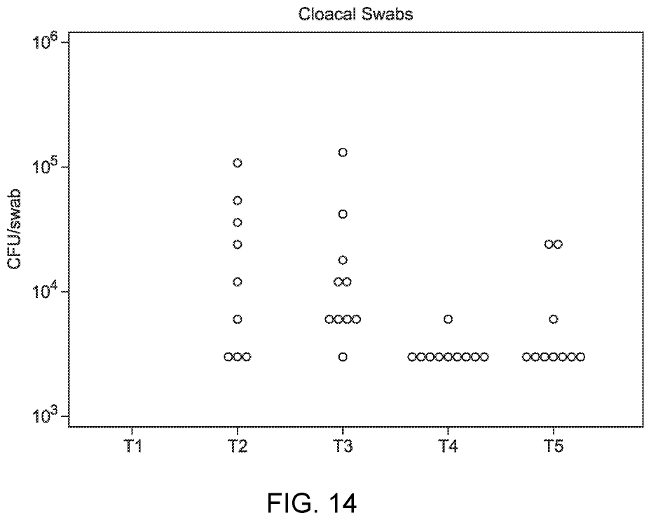

FIG. 14. Mean log 10 CFU counts of Campylobacter from cloacal swabs in groups treated with different anti-Campylobacter vaccine candidates.

In conclusion, L. reuteri delivering anti-flagellar nanobodies significantly reduced Campylobacter load.

### Example 10

### In Vitro Evaluation of Cj11, Cj12 and Cj14 for Efficacy Using a Soft Agar Motility Assay

To understand the relative contributions of CJ11, CJ12 and CJ14 to efficacy observed in vivo, recombinant CJ11, CJ12 and CJ14 were evaluated for their ability to inhibit Campylobacter motility using a soft agar motility assay. More specifically, C. jejuni strains obtained from various geographical regions within United States (US) and European Union (EU) were streaked for single colony isolation on Mueller Hinton (MH) agar (BD Difco, 225250) and incubated overnight under microaerophilic conditions at 42° C. (5% O2, 10% CO2, 80% relative humidity).

The following day 20 mL liquid culture was inoculated with 5 colonies from the streak plate and incubated overnight with agitation of 100 rpm. C. jejuni strains were prepared by diluting the C. jejuni strains to an OD600 of 0.05±0.02 with warmed MH broth and aliquoting 100 μL of the diluted culture to a sterile microcentrifuge tube. Approximately, 1 μg of nanobody to be screened was added to the bacterial aliquot and pipette mixed. For each strain evaluated, a PBS control was prepared by adding PBS equivalent to the volume of nanobody added to the experimental samples. The samples were incubated for 30 minutes at room temperature using a tube rotator (Fisherbrand 88861051) at setting 15. After incubation, 2 μL of sample was stabbed into MH soft agar (0.4% agar) and incubated overnight (16-18 h) under microaerophilic conditions at 42° C. (5% 02, 10% CO2, 80% relative humidity). The effect of nanobodies against motility was evaluated by measuring the diameter of the bacterial growth for each sample and control. Reduction in the diameter of the nanobody treated samples in comparison to the PBS control sample within strains suggested inhibition of motility by the nanobody.

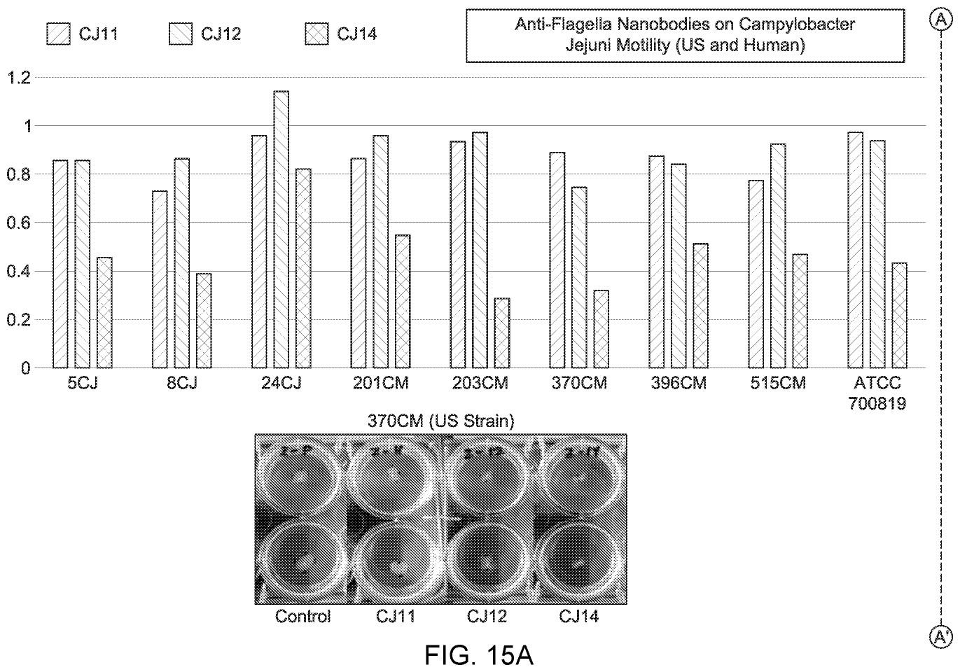

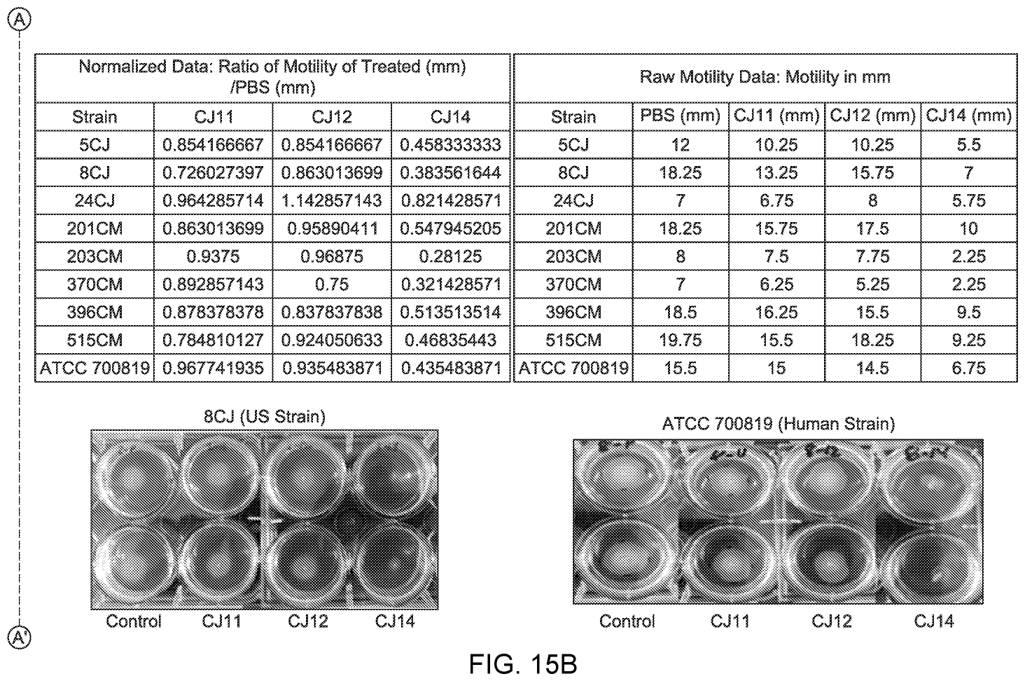

In conclusion, as shown in FIGS. 15A and B and 16A and B, CJ14 showed significant inhibition of motility of all the US and EU strains tested in this study.

This invention may be embodied in other forms or carried out in other ways without departing from the spirit or essential characteristics thereof. The present disclosure is therefore to be considered as in all aspects illustrated and not restrictive, the scope of the invention being indicated by the description hereof, and all changes which come within the meaning and range of equivalency are intended to be embraced therein.

Various references are cited throughout this Specification, each of which is incorporated herein by reference in its entirety.

1. A method for reducing Campylobacter colonization or infection in an animal in need thereof, the method comprising administering to said animal an effective amount of a composition comprising a polypeptide encoding a nanobody selected from the group consisting of SEQ ID NO: 29 (CfrA), SEQ ID NO: 91 (CfrA), SEQ ID NO: 69 (CadF), SEQ ID NO: 82 (CadF), SEQ ID NO: 49 (CjaD), SEQ ID NO: 84 (CjaD), SEQ ID NO: 66 (CmeC), SEQ ID NO:78 (CmeC), SEQ ID NO: 53 (flagella), SEQ ID NO: 85 (flagella), SEQ ID NO: 73 (flagella), SEQ ID NO: 86 (flagella), SEQ ID NO: 74 (flagella), SEQ ID NO: 87 (flagella), SEQ ID NO: 75 (flagella), SEQ ID NO: 80 (flagella), SEQ ID NO: 76 (flagella), SEQ ID NO: 89 (flagella), SEQ ID NO: 77 (flagella), SEQ ID NO: 90 (flagella), and combinations thereof.

2. The method according to claim 1, wherein the Campylobacter jejuni.

3. The method according to claim 1, wherein the animal is a chicken.

4. The method according to claim 1, wherein the animal is a human.

5. A method for improving food safety in food products consumed by animals, comprising adding a composition comprising a polypeptide encoding a nanobody selected from the group consisting of SEQ ID NO: 29 (CfrA), SEQ ID NO: 91 (CfrA), SEQ ID NO: 69 (CadF), SEQ ID NO: 82 (CadF), SEQ ID NO: 49 (CjaD), SEQ ID NO: 84 (CjaD), SEQ ID NO:66 (CmeC), SEQ ID NO:78 (CmeC), SEQ ID NO: 53 (flagella), SEQ ID NO: 85 (flagella), SEQ ID NO: 73 (flagella), SEQ ID NO: 86 (flagella), SEQ ID NO: 74 (flagella), SEQ ID NO: 87 (flagella), SEQ ID NO: 75 (flagella), SEQ ID NO: 80 (flagella), SEQ ID NO: 76 (flagella), SEQ ID NO: 89 (flagella), SEQ ID NO: 77 (flagella), SEQ ID NO: 90 (flagella), and combinations thereof, to said food products prior to ingesting.

6. (canceled)

7. (canceled)

8. (canceled)

9. A method for treating bacterial gastroenteritis in an animal in need thereof, the method comprising administering an effective amount of a composition comprising a polypeptide encoding a nanobody selected from the group consisting of SEQ ID NO: 29 (CfrA), SEQ ID NO: 91 (CfrA), SEQ ID NO: 69 (CadF), SEQ ID NO: 82 (CadF), SEQ ID NO: 49 (CjaD), SEQ ID NO: 84 (CjaD), SEQ ID NO:66 (CmeC), SEQ ID NO:78 (CmeC), SEQ ID NO: 53 (flagella), SEQ ID NO: 85 (flagella), SEQ ID NO: 73 (flagella), SEQ ID NO: 86 (flagella), SEQ ID NO: 74 (flagella), SEQ ID NO: 87 (flagella), SEQ ID NO: 75 (flagella), SEQ ID NO: 80 (flagella), SEQ ID NO: 76 (flagella), SEQ ID NO: 89 (flagella), SEQ ID NO: 77 (flagella), SEQ ID NO: 90 (flagella), and combinations thereof, to said animal.

10. (canceled)

11. (canceled)

12. (canceled)

13. A method for inducing an immune response to Campylobacter bacteria in an animal in need thereof, comprising administering to said animal a composition comprising a polypeptide encoding a nanobody selected from the group consisting of SEQ ID NO: 29 (CfrA), SEQ ID NO: 91 (CfrA), SEQ ID NO: 69 (CadF), SEQ ID NO: 82 (CadF), SEQ ID NO: 49 (CjaD), SEQ ID NO: 84 (CjaD), SEQ ID NO:66 (CmeC), SEQ ID NO:78 (CmeC), SEQ ID NO: 53 (flagella), SEQ ID NO: 85 (flagella), SEQ ID NO: 73 (flagella), SEQ ID NO: 86 (flagella), SEQ ID NO: 74 (flagella), SEQ ID NO: 87 (flagella), SEQ ID NO: 75 (flagella), SEQ ID NO: 80 (flagella), SEQ ID NO: 76 (flagella), SEQ ID NO: 89 (flagella), SEQ ID NO: 77 (flagella), SEQ ID NO: 90 (flagella), and combinations thereof.

14. (canceled)

15. (canceled)

16. (canceled)

17. A composition comprising a polypeptide encoding a nanobody directed to one or more Campylobacter antigens selected from CmeC, CadF, CfrA, CjaD, and flagella, and said nanobody is selected from the group consisting of SEQ ID NO: 29 (CfrA), SEQ ID NO: 91 (CfrA), SEQ ID NO: 69 (CadF), SEQ ID NO: 82 (CadF), SEQ ID NO: 49 (CjaD), SEQ ID NO: 84 (CjaD), SEQ ID NO:66 (CmeC), SEQ ID NO:78 (CmeC), SEQ ID NO: 53 (flagella), SEQ ID NO: 85 (flagella), SEQ ID NO: 73 (flagella), SEQ ID NO: 86 (flagella), SEQ ID NO: 74 (flagella), SEQ ID NO: 87 (flagella), SEQ ID NO: 75 (flagella), SEQ ID NO: 80 (flagella), SEQ ID NO: 76 (flagella), SEQ ID NO: 89 (flagella), SEQ ID NO: 77 (flagella), SEQ ID NO: 90 (flagella), and combinations thereof.

18. The composition of claim 17, wherein said composition is a pharmaceutical composition comprising said nanobody and a carrier.

19. The composition of claim 17, wherein said composition comprises a recombinant host cell containing a nucleic acid vector encoding and expressing said nanobodies.

20. The composition of claim 17, wherein said recombinant host cell is a recombinant prokaryotic cell selected from the group consisting of Bacillus, Lactobacillus, Lactococcus, Enterococcus, Pseudomonas, and Streptomyces.

21. The composition of claim 20, wherein said recombinant host cell is a strain of Lactobacillus reuteri.

22. The composition of claim 20, wherein said recombinant host cell is a strain of E. coli.

23. The composition of claim 17, wherein said recombinant host cell is a recombinant eukaryotic cell selected from the group consisting of fungi, animal cells, and plant cells.

24. The composition of claim 17, wherein said combination comprises polypeptides encoding a nanobody directed to Campylobacter antigens selected from CmeC, CadF, and flagella, and said nanobody combination is selected from the group consisting of SEQ ID NOs: 77, 66, and 69, SEQ ID NOs: 77, 78, and 69, SEQ ID NOs: 77, 66, and 82, SEQ ID NOs: 77, 78, and 82, SEQ ID NOs: 90, 66, and 69, SEQ ID NOs: 90, 78, and 69, SEQ ID NOs: 90, 66, and 82, or SEQ ID NOs: 90, 78, and 82.

25. The composition of claim 19, wherein said recombinant host cell contains a nucleic acid vector encoding and expressing a combination of polypeptides encoding nanobodies of SEQ ID NOs: 77, 66, and 69, SEQ ID NOs: 77, 78, and 69, SEQ ID NOs: 77, 66, and 82, SEQ ID NOs: 77, 78, and 82, SEQ ID NOs: 90, 66, and 69, SEQ ID NOs: 90, 78, and 69, SEQ ID NOs: 90, 66, and 82, or SEQ ID NOs: 90, 78, and 82.

26. The composition of claim 25, wherein said recombinant host cell comprises a strain of Lactobacillus reuteri.

27. The method of claim 1, wherein said combination comprises polypeptides encoding a nanobody directed to Campylobacter antigens selected from CmeC, CadF, and flagella, and said nanobody combination is selected from the group consisting of SEQ ID NOs: 77, 66, and 69, SEQ ID NOs: 77, 78, and 69, SEQ ID NOs: 77, 66, and 82, SEQ ID NOs: 77, 78, and 82, SEQ ID NOs: 90, 66, and 69, SEQ ID NOs: 90, 78, and 69, SEQ ID NOs: 90, 66, and 82, or SEQ ID NOs: 90, 78, and 82.

28. A nucleic encoding a nanobody polypeptide directed to one or more Campylobacter antigens selected from CmeC, CadF, CfrA, CjaD, and flagella selected from the group consisting of SEQ ID NO: 29 (CfrA), SEQ ID NO: 91 (CfrA), SEQ ID NO: 69 (CadF), SEQ ID NO: 82 (CadF), SEQ ID NO: 49 (CjaD), SEQ ID NO: 84 (CjaD), SEQ ID NO:66 (CmeC), SEQ ID NO:78 (CmeC), SEQ ID NO: 53 (flagella), SEQ ID NO: 85 (flagella), SEQ ID NO: 73 (flagella), SEQ ID NO: 86 (flagella), SEQ ID NO: 74 (flagella), SEQ ID NO: 87 (flagella), SEQ ID NO: 75 (flagella), SEQ ID NO: 80 (flagella), SEQ ID NO: 76 (flagella), SEQ ID NO: 89 (flagella), SEQ ID NO: 77 (flagella), SEQ ID NO: 90 (flagella), and combinations thereof.

29. The nucleic acid encoding a nanobody polypeptide acid of claim 28, comprising a combination of polypeptides encoding a nanobody directed to Campylobacter antigens selected from CmeC, CadF, and flagella, and said nanobody polypeptide combination is selected from the group consisting of SEQ ID NOs: 77, 66, and 69, SEQ ID NOs: 77, 78, and 69, SEQ ID NOs: 77, 66, and 82, SEQ ID NOs: 77, 78, and 82, SEQ ID NOs: 90, 66, and 69, SEQ ID NOs: 90, 78, and 69, SEQ ID NOs: 90, 66, and 82, or SEQ ID NOs: 90, 78, and 82 SEQ ID NOs: 77, 66, and 69, SEQ ID NOs: 77, 78, and 69, SEQ ID NOs: 77, 66, and 82, SEQ ID NOs: 77, 78, and 82, SEQ ID NOs: 90, 66, and 69, SEQ ID NOs: 90, 78, and 69, SEQ ID NOs: 90, 66, and 82, or SEQ ID NOs: 90, 78, and 82.
